(19) United States
(12) Patent Application Publication
(10) Pub. No.: US 2026/0250729 A1
(43) Pub. Date: Aug. 27, 2026

(54) METHODS OF PRODUCING MORPHINAN ALKALOIDS AND DERIVATIVES

(71) Applicant: Antheia, Inc., Menlo Park, CA (US)

(72) Inventors: Christina D. SMOLKE, Menlo Park, CA (US); Isis TRENCHARD, Redwood City, CA (US); Kristy M. HAWKINS, Menlo Park, CA (US); Catherine THODEY, Menlo Park, CA (US)

(21) Appl. No.: 19/338,793

(22) Filed: Sep. 24, 2025

Related U.S. Application Data
(63) Continuation of application No. 18/511,394, filed on Nov. 16, 2023, now Pat. No. 12,674,185.
Application No. 18/511,394 is a continuation of application No. 16/984,900, filed on Aug. 4, 2020, now Pat. No. 11,884,949.
Application No. 16/984,900 is a continuation of application No. PCT/US19/17357, filed on Feb. 8, 2019.
(60) Provisional application No. 62/628,264, filed on Feb. 8, 2018.

(51) Int. Cl. C12P 17/12; C12N 9/88; C12N 9/90; C12N 15/52

(52) U.S. Cl. CPC C12P 17/12; C12N 9/88; C12N 9/90; C12N 15/52; C12Y 402/99

(57) ABSTRACT

A method of producing promorphinan, morphinan, nal-opioid, and nor-opioid alkaloid products through the increased conversion of a promorphinan alkaloid to a morphinan alkaloid. The method comprises contacting the promorphinan alkaloid with at least one enzyme. Contacting the promorphinan alkaloid with the at least one enzyme converts the promorphinan alkaloid to a Morphinan alkaloid.

### CROSS-REFERENCE TO RELATED APPLICATIONS

This application is a continuation of U.S. application Ser. No. 18/511,394, filed Nov. 16, 2023, which is a continuation application of U.S. application Ser. No. 16/984,900, filed Aug. 4, 2020, which is a continuation of International Application No. PCT/US19/17357, filed Feb. 8, 2019, which claims the benefit of U.S. Provisional Application No. 62/628,264, which was filed Feb. 8, 2018, which are herein incorporated by reference in their entireties, including any drawings.

### INCORPORATION OF SEQUENCE LISTING

This application incorporates by reference a Sequence Listing named “2023-11-20 Revised_Sequence_Listing_ST26 062074-511C02US.xml” which is 291,567 bytes in size, and replaces the original Sequence Listing file that was initially submitted with the application.

### SUMMARY OF THE INVENTION

The present disclosure provides methods for the production of diverse benzylisoquinoline alkaloids (BIAs) in engineered host cells. The present disclosure further provides compositions of diverse alkaloids produced in engineered host cells. Additionally, the present disclosure provides methods for the production of a thebaine synthase in engineered host cells. In particular cases, the disclosure provides methods for producing diverse alkaloid products through the conversion of a promorphinan alkaloid into a morphinan alkaloid in an engineered host cell. In further particular cases, the present disclosure provides methods for producing diverse alkaloid products through the conversion of salutaridinol-7-O-acetate to thebaine.

An aspect of the invention provides an engineered non-plant cell having increased tyrosine hydroxylase activity relative to a non-engineered cell. Another aspect of the invention provides an engineered non-plant cell having increased tyrosine hydroxylase activity relative to a cell that expresses wild-type TyrH. An additional aspect of the invention provides an engineered non-plant cell having increased tyrosine hydroxylase activity relative to a cell that expresses wild-type TyrH without mutations that increase tyrosine hydroxylase activity as provided herein. In particular, the engineered non-plant cell has at least one modification selected from a group consisting of: a substrate inhibition alleviating mutation; a product inhibition alleviating mutation; and a cofactor recovery promoting mechanism.

An aspect of the invention provides an engineered plant cell having increased tyrosine hydroxylase activity relative to a non-engineered cell. Another aspect of the invention provides an engineered plant cell having increased tyrosine hydroxylase (TyrH) activity relative to a cell that expresses wild-type TyrH. An additional aspect of the invention provides an engineered plant cell having increased tyrosine hydroxylase activity relative to a cell that expresses wild-type TyrH without mutations that increase tyrosine hydroxylase activity as provided herein. In particular, the engineered plant cell has at least one modification selected from a group consisting of: a substrate inhibition alleviating mutation; a product inhibition alleviating mutation; and a cofactor recovery promoting mechanism.

In some embodiments, the disclosure provides methods for increasing production of diverse alkaloid products through the epimerization of a (S)-1-benzylisoquinoline alkaloid to a (R)-1-benyzlisoquinoline alkaloid via engineered epimerases in an engineered host cell. In further embodiments, the present disclosure provides methods for increasing production of diverse alkaloid products through the epimerization of (S)-reticuline to (R)-reticuline via an engineered epimerase comprising two separate enzymes encoding an oxidase and a reductase compared to the production of diverse alkaloid products through the epimerization of (S)-reticuline to (R)-reticuline via a wild-type epimerase.

While engineered split epimerases may be composed of a separate oxidase enzyme and reductase enzyme that originate from a parent or wild-type epimerase, engineered epimerases may also comprise a separate oxidase enzyme and reductase enzyme that originate from separate parent or wild-type epimerases. Examples of parent epimerases having an oxidase and reductase component comprise amino acid sequences selected from the group consisting of: SEQ ID NOs: 1, 2, 3, 4, 5, 6, 7, 8, 9, 10, 11, 12, 13, 14, 15, and 16, as listed in Table 1.

In some embodiments, the disclosure provides methods for increasing production of diverse alkaloid products through the conversion of a promorphinan alkaloid to a morphinan alkaloid via thebaine synthases in an engineered host cell. In further embodiments, the present disclosure provides methods for increasing production of diverse alkaloid products through the conversion of salutaridinol-7-O-acetate to thebaine via a thebaine synthase. Examples of parent thebaine synthases comprise amino acid sequences selected from the group consisting of: SEQ ID NOs: 30, 31, 32, 33, 34, 35, 36, and 37 as listed in Table 2.

In some embodiments, the disclosure provides methods for increasing production of diverse alkaloid products through the conversion of a promorphinan alkaloid to a morphinan alkaloid via engineered thebaine synthases in an engineered host cell. In further embodiments, the present disclosure provides methods for increasing production of diverse alkaloid products through the conversion of salutaridinol-7-O-acetate to thebaine via an engineered thebaine synthase.

In some embodiments, the engineered thebaine synthase is a fusion enzyme. In further embodiments, the thebaine synthase is fused to an acetyl transferase enzyme. In further embodiments, the thebaine synthase is encoded within an acetyl transferase enzyme. In other embodiments, the thebaine synthase is fused to a reductase enzyme.

In some examples, an engineered non-plant cell comprises a plurality of coding sequences each encoding an enzyme that is selected from the group of enzymes listed in Table 3. In some examples, the heterologous coding sequences may be operably connected. Heterologous coding sequences that are operably connected may be within the same pathway of producing a particular benzylisoquinoline alkaloid product via a thebaine synthase activity or an engineered thebaine synthase activity.

In some embodiments this disclosure provides a method of converting a tetracyclic promorphinan precursor to a thebaine, comprising contacting the tetracyclic promorphinan precursor with at least one enzyme, wherein contacting the tetracyclic promorphinan precursor with the at least one enzyme converts the tetracyclic promorphinan precursor to a thebaine. In some cases, the at least one enzyme is produced by culturing an engineered non-plant cell having a coding sequence for encoding the at least one enzyme. In some cases, the method further comprises adding a tetracyclic promorphinan precursor to the cell culture. In some cases, the method further comprises recovering the thebaine, or a derivative thereof, from the cell culture. In some cases, the at least one enzyme comprises a thebaine synthase. In some cases, the thebaine synthase comprises an amino acid sequence selected from the group consisting of: SEQ ID NOs: 30, 31, 32, 33, 34, 35, 36, and 37. In some cases, the thebaine synthase enzyme is a Bet v 1 fold protein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the invention will be obtained by reference to the following detailed description that sets forth illustrative embodiments, in which the principles of the invention are utilized, and the accompanying drawings of which:

FIG. 1 illustrates examples of synthesis, recycling, and salvage pathways of tetrahydrobiopterin, in accordance with embodiments of the invention.

FIG. 2 illustrates a biosynthetic scheme for conversion of glucose to 4-HPA, dopamine, and 3,4-DHPA, in accordance with embodiments of the invention.

FIG. 3 illustrates a schematic example of (R)-1-benzylisoquinoline alkaloid formation, in accordance with embodiments of the invention.

FIG. 4 illustrates an amino acid sequence of a parent DRS-DRR enzyme, in accordance with embodiments of the invention.

FIG. 5 illustrates amino acid sequences of a DRS enzyme and a DRR enzyme, respectively, that are derived from a parent fusion enzyme illustrated in FIG. 4, in accordance with embodiments of the invention.

FIG. 6 illustrates an enzyme having opioid 3-O-demethylase activity, in accordance with embodiments of the invention.

FIG. 7 illustrates an enzyme having opioid N-demethylase activity, in accordance with embodiments of the invention.

FIG. 8 illustrates an enzyme having N-methyltransferase activity, in accordance with embodiments of the invention.

FIG. 9 illustrates the functional expression of BM3 variants, in accordance with embodiments of the invention.

FIG. 10 illustrates a biosynthesis scheme for conversion of L-tyrosine to a nor-opioid or nal-opioid in a microbial cell, in accordance with embodiments of the invention.

FIG. 11 illustrates plasmid/YAC vectors for enzyme expression and engineering, in accordance with embodiments of the invention.

FIG. 12 illustrates a biosynthetic scheme for conversion of L-tyrosine to reticuline via norcoclaurine, in accordance with embodiments of the invention.

FIG. 13 illustrates a biosynthetic scheme for conversion of L-tyrosine to reticuline via norlaudanosoline, in accordance with embodiments of the invention.

FIG. 14 illustrates a biosynthetic scheme for conversion of L-tyrosine to morphinan alkaloids, in accordance with embodiments of the invention.

FIG. 15 illustrates a biosynthetic scheme for production of semi-synthetic opioids, in accordance with embodiments of the invention.

FIG. 16 illustrates a biosynthetic scheme for production of opioids, in accordance with embodiments of the invention.

FIG. 17 illustrates an alignment between PbDRS-DRR, PrDRS, and PrDRR, in accordance with embodiments of the invention.

FIG. 18 illustrates yeast platform strains for the production of reticuline from L-tyrosine, in accordance with embodiments of the invention.

FIG. 19 illustrates yeast strains for the production of thebaine and hydrocodone from L-tyrosine, in accordance with embodiments of the invention.

FIG. 20 illustrates the general ring closure reaction converting a tetracyclic scaffold to a pentacyclic scaffold, in accordance with embodiments of the invention.

FIG. 21-1 illustrates a phylogenetic tree generated through a bioinformatic search for morphinan alkaloid generating enzymes, in accordance with embodiments of the invention.

FIG. 21-2 illustrates a phylogenetic tree generated through a bioinformatic search for morphinan alkaloid generating enzymes, in accordance with embodiments of the invention.

FIG. 21-3 illustrates a phylogenetic tree generated through a bioinformatic search for morphinan alkaloid generating enzymes, in accordance with embodiments of the invention.

FIG. 21-4 illustrates a phylogenetic tree generated through a bioinformatic search for morphinan alkaloid generating enzymes, in accordance with embodiments of the invention.

FIG. 22 illustrates the production of the morphinan alkaloid thebaine from sugar and L-tyrosine from an engineered yeast strain, in accordance with embodiments of the invention.

FIG. 23 illustrates the production of promorphinan alkaloids and a morphinan alkaloid thebaine from sugar and L-tyrosine from an engineered yeast strain, in accordance with embodiments of the invention.

### DETAILED DESCRIPTION OF THE INVENTION

The present disclosure provides methods for the production of diverse benzylisoquinoline alkaloids (BIAs) in engineered host cells. The present disclosure further provides compositions of diverse alkaloids produced in engineered host cells. Additionally, the present disclosure provides methods for the production of a thebaine synthase in engineered host cells. Additionally, the present disclosure provides methods for the production of an engineered thebaine synthase in engineered host cells. In particular cases, the disclosure provides methods for producing promorphinan, morphinan, nal-opioid and nor-opioid alkaloid products through the increased conversion of a promorphinan alkaloid to a morphinan alkaloid in an engineered host cell. In further particular cases, the disclosure provides methods for producing morphinan, nal-opioid and nor-opioid alkaloid products through the increased conversion of a promorphinan alkaloid to a morphinan alkaloid in an engineered host cell. In further particular cases, the present disclosure provides methods for producing diverse alkaloid products through the increased conversion of a promorphinan alkaloid to a morphinan alkaloid.

### Benzylisoquinoline Alkaloids (BIAs) of Interest

Host cells which produce BIAs of interest are provided. In some examples, engineered strains of host cells such as the engineered strains of embodiments discussed herein may provide a platform for producing benzylisoquinoline alkaloids of interest and modifications thereof across several structural classes including, but not limited to, precursor BIAs, benzylisoquinolines, promorphinans, morphinans, nal-opioids, nor-opioids, and others. Each of these classes may include biosynthetic precursors, intermediates, and metabolites thereof, of any convenient member of an engineered host cell biosynthetic pathway that may lead to a member of the class. Non-limiting examples of compounds are given below for each of these structural classes. In some cases, the structure of a given example may or may not be characterized itself as a benzylisoquinoline alkaloid. In some cases, the present chemical entities may include all possible isomers, including single enantiomers, racemic mixtures, optically pure forms, mixtures of diastereomers, and intermediate mixtures.

BIA precursors may include, but are not limited to, norcoclaurine (NC) and norlaudanosoline (NL), as well as NC and NL precursors, such as tyrosine, tyramine, 4-hydroxyphenylacetaldehyde (4-HPA), 4-hydroxyphenylpyruvic acid (4-HPPA), L-3,4-dihydroxyphenylalanine (L-DOPA), 3,4-dihydroxyphenylacetaldehyde (3,4-DHPA), and dopamine. In some embodiments, the one or more BIA precursors are 3,4-dihydroxyphenylacetaldehyde (3,4-DHPA) and dopamine. In certain instances, the one or more BIA precursors are 4-hydroxyphenylacetaldehyde (4-HPA) and dopamine. In particular, NL and NC may be synthesized, respectively, from precursor molecules via a Pictet-Spengler condensation reaction, where the reaction may occur spontaneously or may by catalyzed by any convenient enzymes.

Benzylisoquinolines may include, but are not limited to, norcoclaurine, norlaudanosoline, coclaurine, 3′-hydroxycoclaurine, 4′-O-methylnorlaudanosoline, 4′-O-methyl-laudanosoline, N-methylnorcoclaurine, laudanosoline, N-methylcoclaurine, 3′-hydroxy-N-methylcoclaurine, reticuline, norreticuline, papaverine, laudanine, laudanosine, tetrahydropapaverine, 1,2-dihydropapaverine, and orientaline.

Promorphinans may include, but are not limited to, salutaridine, salutaridinol, and salutaridinol-7-O-acetate.

Morphinans may include, but are not limited to, thebaine, codeinone, codeine, morphine, morphinone, oripavine, neopinone, neopine, neomorphine, hydrocodone, dihydrocodeine, 14-hydroxycodeinone, oxycodone, 14-hydroxycodeine, morphinone, hydromorphone, dihydromorphine, dihydroetorphine, ethylmorphine, etorphine, metopon, buprenorphine, pholcodine, heterocodeine, and oxymorphone. In particular, thebaine may be synthesized from salutaridinol-7-O-acetate, where the reaction may occur spontaneously or may be catalyzed by any convenient enzymes.

Nal-opioids may include, but are not limited to, naltrexone, naloxone, nalmefene, nalorphine, nalorphine, nalodeine, naldemedine, naloxegol, 6β-naltrexol, naltrindole, methylnaltrexone, methylsamidorphan, alvimopan, axelopran, bevenpran, dinicotinate, levallorphan, samidorphan, buprenorphine, dezocine, eptazocine, butorphanol, levorphanol, nalbuphine, pentazocine, phenazocine, norbinaltorphimine, and diprenorphine.

Nor-opioids may include, but are not limited to, norcodeine, noroxycodone, northebaine, norhydrocodone, nordihydro-codeine, nor-14-hydroxy-codeine, norcodeinone, nor-14-hydroxy-codeinone, normorphine, noroxymorphone, nororipavine, norhydro-morphone, nordihydro-morphine, nor-14-hydroxy-morphine, normorphinone, and nor-14-hydroxy-morphinone.

In certain embodiments, the engineered strains of the invention may provide a platform for producing compounds related to tetrahydrobiopterin synthesis including, but not limited to, dihydroneopterin triphosphate, 6-pyruvoyl tetrahydropterin, 5,6,7,8-tetrahydrobiopterin, 7,8-dihydrobiopterin, tetrahydrobiopterin 4a-carbinolamine, quinonoid dihydrobiopterin, and biopterin.

### Host Cells

Any convenient cells may be utilized in the subject host cells and methods. In some cases, the host cells are non-plant cells. In some instances, the host cells may be characterized as microbial cells. In certain cases, the host cells are insect cells, mammalian cells, bacterial cells, or yeast cells. Any convenient type of host cell may be utilized in producing the subject BIA-producing cells, see, e.g., US2008/0176754, and US2014/0273109 the disclosures of which are incorporated by reference in their entirety. Host cells of interest include, but are not limited to, bacterial cells, which may be either Gram positive bacterial cells or Gram negative bacterial cells, insect cells such as Drosophila melanogaster S2 and Spodoptera frugiperda Sf9 cells, and yeast cells such as Saccharomyces cerevisiae, Schizosaccharomyces pombe, and Pichia pastoris cells. Non-limiting examples of bacterial cells include Bacillus subtilis, Escherichia coli, Streptomyces, Anabaena, Arthrobacter, Acetobacter, Acetobacterium, Bacillus, Bifidobacterium, Brachybacterium, Brevibacterium, Carnobacterium, Clostridium, Corynebacterium, Enterobacter, Escherichia, Gluconacetobacter, Gluconobacter, Hafnia, Halomonas, Klebsiella, Kocuria, Lactobacillus, Leucononstoc, Macrococcus, Methylomonas, Methylobacter, Methylocella, Methylococcus, Microbacterium, Micrococcus, Microcystis, Moorella, Oenococcus, Pediococcus, Prochlorococcus, Propionibacterium, Proteus, Pseudoalteromonas, Pseudomonas, Psychrobacter, Rhodobacter, Rhodococcus, Rhodopseudomonas, Serratia, Staphylococcus, Streptococcus, Streptomyces, Synechococcus, Synechocystis, Tetragenococcus, Weissella, Zymomonas, and Salmonella typhimuium cells. In some examples, the host cells are yeast cells or E. coli cells. In some cases, the host cells are yeast cells or E. coli cells. In some cases, the host cell is a yeast cell. In some instances, the host cell is from a strain of yeast engineered to produce a BIA of interest, such as a morphinan alkaloid. In some instances, the host cell is from a strain of yeast engineered to produce an enzyme of interest. In some instances, the host cell is from a strain of yeast engineered to produce a thebaine synthase.

The thebaine synthase may be able to more efficiently convert a salutaridinol-7-O-acetate to a thebaine relative to a spontaneous reaction. In some instances, the host cell is from a strain of yeast engineered to produce an engineered thebaine synthase. In some embodiments, an engineered thebaine synthase may be an engineered fusion enzyme. Additionally, the engineered thebaine synthase may be able to more efficiently convert a salutaridinol-7-O-acetate to a thebaine relative to a thebaine synthase. In some embodiments, the thebaine synthase may be a wild-type thebaine synthase. In some embodiments, a thebaine synthase may be substantially similar to a wild-type thebaine synthase. In some cases, a thebaine synthase that is substantially similar to a wild-type thebaine synthase may have an amino acid sequence that is at least 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more similar to an amino acid sequence of a wild-type thebaine synthase. The engineered thebaine synthase may be engineered as a fusion enzyme to another enzyme to more efficiently convert a salutaridinol-7-O-acetate to a thebaine relative to the thebaine synthase.

Any of the host cells described in US2008/0176754 and US2014/0273109 by Smolke et al. may be adapted for use in the subject cells and methods. In certain embodiments, the yeast cells may be of the species Saccharomyces cerevisiae (S. cerevisiae). In certain embodiments, the yeast cells may be of the species Schizosaccharomyces pombe. In certain embodiments, the yeast cells may be of the species Pichia pastoris. Yeast is of interest as a host cell because cytochrome P450 proteins are able to fold properly into the endoplasmic reticulum membrane so that their activity is maintained. In examples, cytochrome P450 proteins are involved in some biosynthetic pathways of interest. In additional examples, cytochrome P450 proteins are involved in the production of BIAs of interest. In further examples, cytochrome P450 proteins are involved in the production of an enzyme of interest.

Yeast strains of interest that find use in the invention include, but are not limited to, CEN.PK (Genotype: MATα/α ura3-52 ura3-52/trp1-289/trp1-289 leu2-3_112/leu2-3_112 his3 Δ1/his3 Δ1 MAL2-8C/MAL2-8C SUC2/SUC2), S288C, W303, D273-10B, X2180, A364A, Σ1278B, AB972, SK1, and FL100. In certain cases, the yeast strain is any of S288C (MATα; SUC2 mal mel gal2 CUP1 fio1 flo8-1 hap1), BY4741 (MATα; his3Δ1; leu2Δ0; met15Δ0; ura3Δ0), BY4742 (MATα; his3Δ1; leu2Δ0; lys2Δ0; ura3A0), BY4743 (MATα/MATα; his3Δ1/his3Δ1; leu2Δ0/leu2Δ0; met15Δ0/MET15; LYS2/lys2Δ0; ura3A0/ura3A0), and WAT11 or W(R), derivatives of the W303-B strain (MATα; ade2-1; his3-11, -15; leu2-3, -112; ura3-1; canR; cyr+) which express the Arabidopsis thaliana NADPH-P450 reductase ATR1 and the yeast NADPH-P450 reductase CPR1, respectively. In another embodiment, the yeast cell is W303alpha (MATα; his3-11,15 trp1-1 leu2-3 ura3-1 ade2-1). The identity and genotype of additional yeast strains of interest may be found at EUROSCARF (web.uni-frankfurt.de/fb15/mikro/euroscarf/col_index.html).

In some instances the host cell is a fungal cell. In certain embodiments, the fungal cells may be of the Aspergillus species and strains include Aspergillus Niger (ATCC 1015, ATCC 9029, CBS 513.88), Aspergillus oryzae (ATCC 56747, RIB40), Aspergillus terreus (NIH 2624, ATCC 20542) and Aspergillus nidulans (FGSC A4).

In certain embodiments, heterologous coding sequences may be codon optimized for expression in Aspergillus sp. and expressed from an appropriate promoter. In certain embodiments, the promoter may be selected from phosphoglycerate kinase promoter (PGK), MbfA promoter, cytochrome c oxidase subunit promoter (CoxA), SrpB promoter, TvdA promoter, malate dehydrogenase promoter (MdhA), beta-mannosidase promoter (ManB). In certain embodiments, a terminator may be selected from glucoamylase terminator (GlaA) or TrpC terminator. In certain embodiments, the expression cassette consisting of a promoter, heterologous coding sequence, and terminator may be expressed from a plasmid or integrated into the genome of the host. In certain embodiments, selection of cells maintaining the plasmid or integration cassette may be performed with antibiotic selection such as hygromycin or nitrogen source utilization, such as using acetamide as a sole nitrogen source. In certain embodiments, DNA constructs may be introduced into the host cells using established transformation methods such as protoplast transformation, lithium acetate, or electroporation. In certain embodiments, cells may be cultured in liquid ME or solid MEA (3% malt extract, 0.5% peptone, and +1.5% agar) or in Vogel's minimal medium with or without selection.

In some instances the host cell is a bacterial cell. The bacterial cell may be selected from any bacterial genus. Examples of genuses from which the bacterial cell may come include Anabaena, Arthrobacter, Acetobacter, Acetobacterium, Bacillus, Bifidobacterium, Brachybacterium, Brevibacterium, Carnobacterium, Clostridium, Corynebacterium, Enterobacter, Escherichia, Gluconacetobacter, Gluconobacter, Hafnia, Halomonas, Klebsiella, Kocuria, Lactobacillus, Leucononstoc, Macrococcus, Methylomonas, Methylobacter, Methylocella, Methylococcus, Microbacterium, Micrococcus, Microcystis, Moorella, Oenococcus, Pediococcus, Prochlorococcus, Propionibacterium, Proteus, Pseudoalteromonas, Pseudomonas, Psychrobacter, Rhodobacter, Rhodococcus, Rhodopseudomonas, Serratia, Staphylococcus, Streptococcus, Streptomyces, Synechococcus, Synechocystis, Tetragenococcus, Weissella, and Zymomonas. Examples of bacterial species which may be used with the methods of this disclosure include Arthrobacter nicotianae, Acetobacter aceti, Arthrobacter arilaitensis, Bacillus cereus, Bacillus coagulans, Bacillus lichenformis, Bacillus pumilus, Bacillus sphaericus, Bacillus stearothermophilus, Bacillus subtilis, Bifidobacterium adolescentis, Brachybacterium tyrofermentans, Brevibacterium linens, Carnobacterium divergens, Corynebacterium flavescens, Enterococcus faecium, Gluconacetobacter europaeus, Gluconacetobacter johannae, Gluconobacter oxydans, Hafnia alvei, Halomonas elongata, Kocuria rhizophila, Lactobacillus acidifarinae, Lactobacillus jensenii, Lactococcus lactis, Lactobacillus yamanashiensis, Leuconostoc citreum, Macrococcus caseolyticus, Microbacterium foliorum, Micrococcus lylae, Oenococcus oeni, Pediococcus acidilactici, Propionibacterium acidipropionici, Proteus vulgaris, Pseudomonas fluorescens, Psychrobacter celer, Staphylococcus condiments, Streptococcus thermophilus, Streptomyces griseus, Tetragenococcus halophilus, Weissella cibaria, Weissella koreensis, Zymomonas mobilis, Corynebacterium glutamicum, Bifidobacterium bifidum/breve/longum, Streptomyces lividans, Streptomyces coelicolor, Lactobacillus plantarum, Lactobacillus sakei, Lactobacillus casei, Pseudoalteromonas citrea, Pseudomonas putida, Clostridium ljungdahlii aceticum acetobutylicum beijerinckii butyricum, and Moorella themocellum/thermoacetica.

In certain embodiments, the bacterial cells may be of a strain of Escherichia coli. In certain embodiments, the strain of E. coli may be selected from BL21 DH5α, XL1-Blue, HB101, BL21, and K12. In certain embodiments, heterologous coding sequences may be codon optimized for expression in E. coli and expressed from an appropriate promoter. In certain embodiments, the promoter may be selected from T7 promoter, tac promoter, trc promoter, tetracycline-inducible promoter (tet), lac operon promoter (lac), lacO1 promoter. In certain embodiments, the expression cassette consisting of a promoter, heterologous coding sequence, and terminator may be expressed from a plasmid or integrated into the genome. In certain embodiments, the plasmid is selected from pUC19 or pBAD. In certain embodiments, selection of cells maintaining the plasmid or integration cassette may be performed with antibiotic selection such as kanamycin, chloramphenicol, streptomycin, spectinomycin, gentamycin, erythromycin or ampicillin. In certain embodiments, DNA constructs may be introduced into the host cells using established transformation methods such as conjugation, heat shock chemical transformation, or electroporation. In certain embodiments, cells may be cultured in liquid Luria-Bertani (LB) media at about 37° C. with or without antibiotics.

In certain embodiments, the bacterial cells may be a strain of Bacillus subtilis. In certain embodiments, the strain of B. subtilis may be selected from 1779, GP25, RO-NN-1, 168, BSn5, BEST195, 1A382, and 62178. In certain embodiments, heterologous coding sequences may be codon optimized for expression in Bacillus sp. and expressed from an appropriate promoter. In certain embodiments, the promoter may be selected from grac promoter, p43 promoter, or trnQ promoter. In certain embodiments, the expression cassette consisting of the promoter, heterologous coding sequence, and terminator may be expressed from a plasmid or integrated into the genome. In certain embodiments, the plasmid is selected from pHP13, pE194, pC194, pHT01, or pHT43. In certain embodiments, integrating vectors such as pDG364 or pDG1730 may be used to integrate the expression cassette into the genome. In certain embodiments, selection of cells maintaining the plasmid or integration cassette may be performed with antibiotic selection such as erythromycin, kanamycin, tetracycline, and spectinomycin. In certain embodiments, DNA constructs may be introduced into the host cells using established transformation methods such as natural competence, heat shock, or chemical transformation. In certain embodiments, cells may be cultured in liquid Luria-Bertani (LB) media at 37° C. or M9 medium plus glucose and tryptophan.

### Genetic Modifications to Host Cells

The host cells may be engineered to include one or more modifications (such as two or more, three or more, four or more, five or more, or even more modifications) that provide for the production of BIAs of interest. Additionally or alternatively, the host cells may be engineered to include one or more modifications (such as two or more, three or more, four or more, five or more, or even more modifications) that provide for the production of enzymes of interest. In some cases, a modification is a genetic modification, such as a mutation, addition, or deletion of a gene or fragment thereof, or transcription regulation of a gene or fragment thereof. As used herein, the term “mutation” refers to a deletion, insertion, or substitution of an amino acid(s) residue or nucleotide(s) residue relative to a reference sequence or motif. The mutation may be incorporated as a directed mutation to the native gene at the original locus. In some cases, the mutation may be incorporated as an additional copy of the gene introduced as a genetic integration at a separate locus, or as an additional copy on an episomal vector such as a 2 or centromeric plasmid. In certain instances, the substrate inhibited copy of the enzyme is under the native cell transcriptional regulation. In some instances, the substrate inhibited copy of the enzyme is introduced with engineered constitutive or dynamic regulation of protein expression by placing it under the control of a synthetic promoter. In some examples, the object of one or more modifications may be a native gene. In some examples, the object of one or more modifications may be a non-native gene. In some examples, a non-native gene may be inserted into a host cell. In further examples, a non-native gene may be altered by one or more modifications prior to being inserted into a host cell.

An engineered host cell may overproduce one or more BIAs of interest. By overproduce is meant that the cell has an improved or increased production of a BIA molecule of interest relative to a control cell (e.g., an unmodified cell). By improved or increased production is meant both the production of some amount of the BIA of interest where the control has no BIA of interest production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some BIA of interest production.

An engineered host cell may overproduce one or more (S)-1-benzylisoquinoline alkaloids. In some cases, the engineered host cell may produce some amount of the (S)-1-benzylisoquinoline alkaloid of interest where the control has no (S)-1-benzylisoquinoline alkaloid production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some (S)-1-benzylisoquinoline alkaloid of interest production.

An engineered host cell may further overproduce one or more (R)-1-benzylisoquinoline alkaloids. In some cases, the engineered host cell may produce some amount of the (R)-1-benzylisoquinoline alkaloid of interest where the control has no (R)-1-benzylisoquinoline alkaloid production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some (R)-1-benzylisoquinoline alkaloid of interest production.

An engineered host cell may further overproduce one or more morphinan alkaloids. In some cases, the engineered host cell may produce some amount of the morphinan alkaloid of interest where the control has no morphinan alkaloid production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some morphinan alkaloid of interest production. An engineered host cell may further overproduce one or more of promorphinan, nor-opioid, or nal-opioid alkaloids.

In some cases, the engineered host cell is capable of producing an increased amount of (R)-reticuline relative to a control host cell that lacks the one or more modifications (e.g., as described herein). In some cases, the engineered host cell having an engineered split epimerase is capable of producing an increased amount of (R)-reticuline relative to a host cell having a fused epimerase. In some cases, the engineered host cell having modifications to an oxidase portion of an engineered epimerase is capable of producing an increased amount of (R)-reticuline relative to a control host cell that lacks the one or more modifications to the oxidase portion of the engineered epimerase. In certain instances, the increased amount of (R)-reticuline is about 10% or more relative to the control host cell, such as about 20% or more, about 30% or more, about 40% or more, about 50% or more, about 60% or more, about 80% or more, about 100% or more, about 2-fold or more, about 5-fold or more, or even about 10-fold or more relative to the control host cell. In some cases, (R)-reticuline is the product of an epimerization reaction within an engineered host cell. In some cases, (R)-reticuline is the product of an epimerization reaction catalyzed by at least one engineered epimerase within an engineered host cell. In these cases, (S)-reticuline may be the substrate of the epimerization reaction.

In some cases, the engineered host cell is capable of producing an increased amount of thebaine relative to a control host cell that lacks the one or more modifications (e.g., as described herein). In some cases, the engineered host cell having a thebaine synthase is capable of producing an increased amount of thebaine relative to a host cell that lacks a thebaine synthase. In some cases, the engineered host cell having an engineered thebaine synthease is capable of producing an increased amount of thebaine relative to a host cell having a non-engineered thebaine synthase (e.g., as described herein). In certain instances, the increased amount of thebaine is about 10% or more relative to the control host cell, such as about 20% or more, about 30% or more, about 40% or more, about 50% or more, about 60% or more, about 80% or more, about 100% or more, about 2-fold or more, about 5-fold or more, or even about 10-fold or more relative to the control host cell. In some cases, thebaine is the product of a thebaine synthase reaction within an engineered host cell. In some cases, thebaine is the product of a thebaine synthase reaction catalyzed by at least one engineered thebaine synthase within an engineered host cell. In these cases, salutaridinol-7-O-acetate may be the substrate of the thebaine synthase reaction.

Additionally, an engineered host cell may overproduce one or more enzymes of interest. By overproduce is meant that the cell has an improved or increased production of an enzyme of interest relative to a control cell (e.g., an unmodified cell). By improved or increased production is meant both the production of some amount of the enzyme of interest where the control has no production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some enzyme of interest production.

An engineered host cell may overproduce one or more DRS-DRR enzymes. In some cases, the engineered host cell may produce some amount of the DRS-DRR enzyme where the control has no DRS-DRR enzyme production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some DRS-DRR enzyme production.

An engineered host cell may overproduce one or more engineered DRS-DRR enzymes. In some cases, the engineered host cell may produce some amount of the engineered DRS-DRR epimerase where the control has no DRS-DRR enzyme production, or where the control has a same level of production of wild-type epimerases in comparison to the engineered host cell, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some DRS-DRR enzyme production. In some cases, an engineered DRS-DRR epimerase may be an engineered split epimerase. In some cases, an engineered DRS-DRR epimerase may be an engineered fused epimerase.

An engineered host cell may further overproduce one or more enzymes that are derived from the DRS-DRR enzyme. In some cases, the engineered host cell may produce some amount of the enzymes that are derived from the DRS-DRR enzyme, where the control has no production of enzymes that are derived from the DRS-DRR enzyme, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some production of enzymes that are derived from the DRS-DRR enzyme.

An engineered host cell may overproduce one or more thebaine synthase enzymes. In some cases, the engineered host cell may produce some amount of the thebaine synthase enzyme where the control has no thebaine synthase enzyme production, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some thebaine synthase enzyme production.

An engineered host cell may overproduce one or more engineered thebaine synthase enzymes. In some cases, the engineered host cell may produce some amount of the engineered thebaine synthase where the control has no thebaine synthase enzyme production, or where the control has a same level of production of wild-type thebaine synthase in comparison to the engineered host cell, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some thebaine synthase enzyme production. In some cases, an engineered thebaine synthase may be an engineered fusion enzyme.

An engineered host cell may further overproduce one or more enzymes that are derived from the thebaine synthase enzyme. In some cases, the engineered host cell may produce some amount of the enzymes that are derived from the thebaine synthase enzyme, where the control has no production of enzymes that are derived from the thebaine synthase enzyme, as well as an increase of about 10% or more, such as about 20% or more, about 30% or more, about 40% or more, about 50% or more, about 60% or more, about 80% or more, about 100% or more, such as 2-fold or more, such as 5-fold or more, including 10-fold or more in situations where the control has some production of enzymes that are derived from the thebaine synthase enzyme.

In some cases, the one or more (such as two or more, three or more, or four or more) modifications may be selected from: a substrate inhibition alleviating mutation in a biosynthetic enzyme gene; a product inhibition alleviating mutation in a biosynthetic enzyme gene; a cofactor recovery promoting mechanism; a feedback inhibition alleviating mutation in a biosynthetic enzyme gene; a transcriptional modulation modification of a biosynthetic enzyme gene; an inactivating mutation in an enzyme gene; an epimerization modification; and a heterologous coding sequence that encodes an enzyme. A cell that includes one or more modifications may be referred to as an engineered cell.

In some cases, the one or more (such as two or more, three or more, or four or more) modifications may be selected from: a localization mutation; a cytochrome P450 reductase interaction mutation; an accessibility mutation; an activity enhancing mutation; an engineered fused thebaine synthase modification, and an engineered split epimerase modification. A cell that includes one or more modifications may be referred to as an engineered cell.

### Substrate Inhibition Alleviating Mutations

In some instances, the engineered host cells are cells that include one or more substrate inhibition alleviating mutations (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “substrate inhibition alleviating mutation” refers to a mutation that alleviates a substrate inhibition control mechanism of the cell.

A mutation that alleviates substrate inhibition reduces the inhibition of a regulated enzyme in the cell of interest relative to a control cell and provides for an increased level of the regulated compound or a downstream biosynthetic product thereof. In some cases, by alleviating inhibition of the regulated enzyme is meant that the IC₅₀ of inhibition is increased by 2-fold or more, such as by 3-fold or more, 5-fold or more, 10-fold or more, 30-fold or more, 100-fold or more, 300-fold or more, 1000-fold or more, or even more. By increased level is meant a level that is 110% or more of that of the regulated compound in a control cell or a downstream product thereof, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the regulated compound in the engineered host cell or a downstream product thereof.

A variety of substrate inhibition control mechanisms and biosynthetic enzymes in the engineered host cell that are directed to regulation of levels of BIAs of interest, or precursors thereof, may be targeted for substrate inhibition alleviation. The engineered host cell may include one or more substrate inhibition alleviating mutations in one or more biosynthetic enzyme genes. The one or more mutations may be located in any convenient biosynthetic enzyme genes where the biosynthetic enzyme is subject to regulatory control. In some embodiments, the one or more biosynthetic enzyme genes encode one or more tyrosine hydroxylase enzymes. In certain instances, the one or more substrate inhibition alleviating mutations are present in a biosynthetic enzyme gene that is TyrH. In some embodiments, the engineered host cell may include one or more substrate inhibition alleviating mutations in one or more biosynthetic enzyme genes such as one of those genes described in Table 3.

In certain embodiments, the one or more substrate inhibition alleviating mutations are present in the TyrH gene. The TyrH gene encodes tyrosine hydroxylase, which is an enzyme that converts tyrosine to L-DOPA. However, TyrH is inhibited by its substrate, tyrosine. Mammalian tyrosine hydroxylase activity, such as that seen in humans or rats, can be improved through mutations to the TyrH gene that relieve substrate inhibition. In particular, substrate inhibition from tyrosine can be relieved by a point mutation W166Y in the TyrH gene. The point mutation W166Y in the TyrH gene may also improve the binding of the cosubstrate of tyrosine hydroxylase, BH₄, to catalyze the reaction of tyrosine to L-DOPA. The mutants of TyrH, when expressed in yeast strains to produce BIAs from sugar (such as those described in U.S. Provisional Patent Application Ser. No. 61/899,496) can significantly improve the production of BIAs.

Any convenient numbers and types of mutations may be utilized to alleviate a substrate inhibition control mechanism. In certain embodiments, the engineered host cells of the present invention may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more substrate inhibition alleviating mutations, such as 1, 2, 3, 4, 5, 6, 7, 8, 9, 10, 11, 12, 13, 14 or 15 substrate inhibition alleviating mutations in one or more biosynthetic enzyme genes within the engineered host cell.

### Cofactor Recovery Promoting Mechanisms

In some instances, the engineered host cells are cells that include one or more cofactor recovery promoting mechanisms (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “cofactor recovery promoting mechanism” refers to a mechanism that promotes a cofactor recovery control mechanism of the cell.

A variety of cofactor recovery control mechanisms and biosynthetic enzymes in the engineered host cell that are directed to regulation of levels of BIAs of interest, or precursors thereof, may be targeted for cofactor recovery promotion. The engineered host cell may include one or more cofactor recovery promoting mechanism in one or more biosynthetic enzyme genes. In examples, the engineered host cell may include a heterologous coding sequence that encodes dihydrofolate reductase (DHFR). When DHFR is expressed, it may convert 7,8-dihydrobiopterin (BH₂) to the tetrahydrobiopterin (BH₄), thereby recovering BH₄ as a TyrH cosubstrate. In some examples, the engineered host cell may include one or more cofactor recovery promoting mechanisms in one or more biosynthetic enzyme genes such as one of those genes described in Table 2.

Any convenient numbers and types of mechanisms may be utilized to promote a cofactor recovery control mechanism. In certain embodiments, the engineered host cells of the present invention may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more cofactor recovery promoting mechanisms such as 1, 2, 3, 4, 5, 6, 7, 8, 9, 10, 11, 12, 13, 14 or 15 cofactor recovery promoting mechanisms in one or more biosynthetic enzyme genes within the engineered host cell.

### Product Inhibition Alleviating Mutations

In some instances, the engineered host cells are cells that include one or more product inhibition alleviating mutations (such as two or more, three or more, four or more, five or more, or even more) in one or more biosynthetic enzyme genes of the cell. In some examples, the one or more biosynthetic enzyme genes are native to the cell (e.g., is present in an unmodified cell). In some examples, the one or more biosynthetic enzyme genes are non-native to the cell. As used herein, the term “product inhibition alleviating mutation” refers to a mutation that alleviates a short term and/or long term product inhibition control mechanism of an engineered host cell. Short term product inhibition is a control mechanism of the cell in which there is competitive binding at a cosubstrate binding site. Long term product inhibition is a control mechanism of the cell in which there is irreversible binding of a compound away from a desired pathway.

A mutation that alleviates product inhibition reduces the inhibition of a regulated enzyme in the cell of interest relative to a control cell and provides for an increased level of the regulated compound or a downstream biosynthetic product thereof. In some cases, by alleviating inhibition of the regulated enzyme is meant that the IC₅₀ of inhibition is increased by 2-fold or more, such as by 3-fold or more, 5-fold or more, 10-fold or more, 30-fold or more, 100-fold or more, 300-fold or more, 1000-fold or more, or even more. By increased level is meant a level that is 110% or more of that of the regulated compound in a control cell or a downstream product thereof, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the regulated compound in the engineered host cell or a downstream product thereof.

A variety of product inhibition control mechanisms and biosynthetic enzymes in the engineered host cell that are directed to regulation of levels of BIAs of interest may be targeted for product inhibition alleviation. The engineered host cell may include one or more product inhibition alleviating mutations in one or more biosynthetic enzyme genes. The mutation may be located in any convenient biosynthetic enzyme genes where the biosynthetic enzyme is subject to regulatory control. In some embodiments, the one or more biosynthetic enzyme genes encode one or more tyrosine hydroxylase enzymes. In certain instances, the one or more product inhibition alleviating mutations are present in a biosynthetic enzyme gene that is TyrH. In some embodiments, the engineered host cell includes one or more product inhibition alleviating mutations in one or more biosynthetic enzyme genes such as one of those genes described in Table 3.

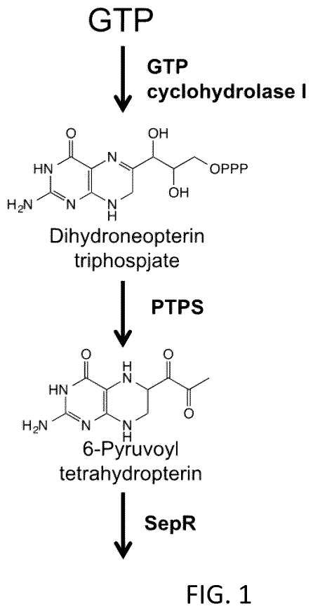

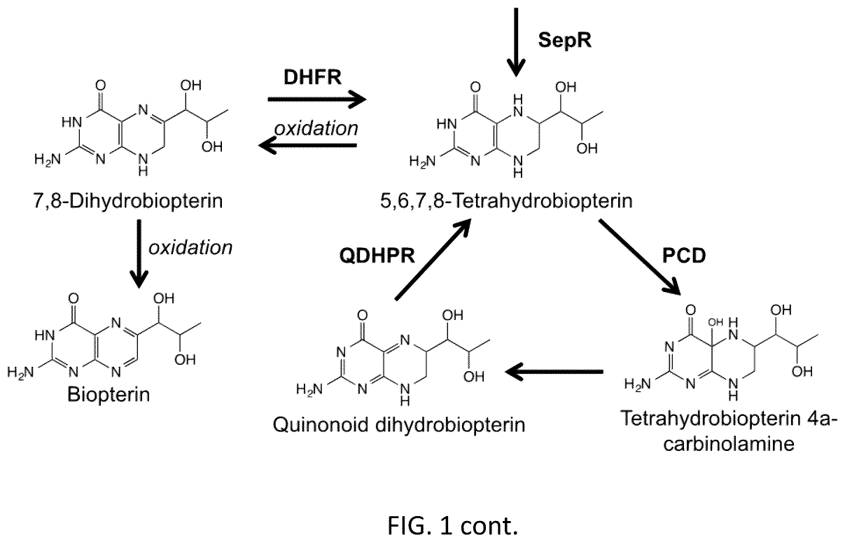

In certain embodiments, the one or more product inhibition alleviating mutations are present in the TyrH gene. The TyrH gene encodes tyrosine hydroxylase, which is an enzyme that converts tyrosine to L-DOPA. TyrH requires tetrahydrobiopterin (BH₄) as a cosubstrate to catalyze the hydroxylation reaction. Some microbial strains, such as Saccharomyces cerevisiae, do not naturally produce BH₄, but can be engineered to produce this substrate through a four-enzyme synthesis and recycling pathway, as illustrated in FIG. 1. FIG. 1 illustrates examples of synthesis, recycling, and salvage pathways of tetrahydrobiopterin, in accordance with embodiments of the invention. FIG. 1 provides the use of the enzymes PTPS, pyruvoyl tetrahydropterin synthase; SepR, sepiapterin reductase; PCD, pterin 4a-carbinolamine dehydratase; QDHPR, dihydropteridine reductase; and DHFR, dihydrofolate reductase. Of the enzymes that are illustrated in FIG. 1, yeast synthesizes an endogenous GTP cyclohydrolase I. GTP and dihydroneopterin triphosphate are naturally synthesized in yeast. Additionally, other metabolites in FIG. 1 are not naturally produced in yeast.

TyrH is inhibited by its product L-DOPA, as well as other catecholamines, particularly dopamine. Mammalian tyrosine hydroxylase activity, such as from humans or rats, can be improved through mutations that relieve product inhibition. For example, short term product inhibition, such as competitive binding at the cosubstrate binding site, can be relieved by a point mutation W166Y on the TyrH gene. In particular, the point mutation W166Y on the TyrH gene may improve binding of the cosubstrate. Additionally, short term product inhibition to relieve competitive binding at the cosubstrate binding site may be improved by a point mutation S40D on the TyrH gene. Short term product inhibition may also be improved by the joint mutations of R37E, R38E on the TyrH gene. In particular, R37E, R38E mutations may together specifically improve tyrosine hydroxylase activity in the presence of dopamine.

Additionally, long term product inhibition may be relieved by point mutations on the TyrH gene. Long term product inhibition relief may include the irreversible binding of catecholamine to iron in the active site such that there is less catecholamine present to act as a product inhibitor of tyrosine hydroxylase activity. Long term product inhibition can be relieved by the mutations E332D and Y371F, respectively, in the TyrH gene.

Combinations of the mutations can be made (such as two or three or more mutations at once) to relieve multiple types of substrate and product inhibition to further improve the activity of TyrH. The mutants of TyrH, when expressed in yeast strains to produce BIAs from sugar (such as those described in U.S. Provisional Patent Application Ser. No. 61/899,496) can significantly improve the production of BIAs.

Any convenient numbers and types of mutations may be utilized to alleviate a product inhibition control mechanism. In certain embodiments, the engineered host cells of the present invention may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more product inhibition alleviating mutations, such as 1, 2, 3, 4, 5, 6, 7, 8, 9, 10, 11, 12, 13, 14 or 15 product inhibition alleviating mutations in one or more biosynthetic enzyme genes within the engineered host cell.

### Feedback Inhibition Alleviating Mutations

In some instances, the engineered host cells are cells that include one or more feedback inhibition alleviating mutations (such as two or more, three or more, four or more, five or more, or even more) in one or more biosynthetic enzyme genes of the cell. In some cases, the one or more biosynthetic enzyme genes are native to the cell (e.g., is present in an unmodified cell). Additionally or alternatively, in some examples the one or more biosynthetic enzyme genes are non-native to the cell. As used herein, the term “feedback inhibition alleviating mutation” refers to a mutation that alleviates a feedback inhibition control mechanism of an engineered host cell. Feedback inhibition is a control mechanism of the cell in which an enzyme in the synthetic pathway of a regulated compound is inhibited when that compound has accumulated to a certain level, thereby balancing the amount of the compound in the cell. A mutation that alleviates feedback inhibition reduces the inhibition of a regulated enzyme in the engineered host cell relative to a control cell. In this way, engineered host cell provides for an increased level of the regulated compound or a downstream biosynthetic product thereof. In some cases, by alleviating inhibition of the regulated enzyme is meant that the IC₅₀ of inhibition is increased by 2-fold or more, such as by 3-fold or more, 5-fold or more, 10-fold or more, 30-fold or more, 100-fold or more, 300-fold or more, 1000-fold or more, or even more. By increased level is meant a level that is 110% or more of that of the regulated compound in a control cell or a downstream product thereof, such as 120% or more, 130% or more, 140% or more, 150% or more, 160% or more, 170% or more, 180% or more, 190% or more, or 200% or more, such as at least 3-fold or more, at least 5-fold or more, at least 10-fold or more or even more of the regulated compound in the host cell or a downstream product thereof.

A variety of feedback inhibition control mechanisms and biosynthetic enzymes that are directed to regulation of levels of BIAs of interest may be targeted for alleviation in the host cell. The host cell may include one or more feedback inhibition alleviating mutations in one or more biosynthetic enzyme genes native to the cell. The one or more mutations may be located in any convenient biosynthetic enzyme genes where the biosynthetic enzyme is subject to regulatory control. In some embodiments, the one or more biosynthetic enzyme genes may encode one or more enzymes selected from a 3-deoxy-d-arabinose-heptulosonate-7-phosphate (DAHP) synthase and a chorismate mutase. In some embodiments, the one or more biosynthetic enzyme genes encode a 3-deoxy-d-arabinose-heptulosonate-7-phosphate (DAHP) synthase. In some instances, the one or more biosynthetic enzyme genes may encode a chorismate mutase. In certain instances, the one or more feedback inhibition alleviating mutations may be present in a biosynthetic enzyme gene selected from ARO4 and ARO7. In certain instances, the one or more feedback inhibition alleviating mutations may be present in a biosynthetic enzyme gene that is ARO4. In certain instances, the one or more feedback inhibition alleviating mutations are present in a biosynthetic enzyme gene that is ARO7. In some embodiments, the engineered host cell may include one or more feedback inhibition alleviating mutations in one or more biosynthetic enzyme genes such as one of those genes described in Table 3.

Any convenient numbers and types of mutations may be utilized to alleviate a feedback inhibition control mechanism. As used herein, the term “mutation” refers to a deletion, insertion, or substitution of an amino acid(s) residue or nucleotide(s) residue relative to a reference sequence or motif. The mutation may be incorporated as a directed mutation to the native gene at the original locus. In some cases, the mutation may be incorporated as an additional copy of the gene introduced as a genetic integration at a separate locus, or as an additional copy on an episomal vector such as a 2 or centromeric plasmid. In certain instances, the feedback inhibited copy of the enzyme is under the native cell transcriptional regulation. In some instances, the feedback inhibited copy of the enzyme is introduced with engineered constitutive or dynamic regulation of protein expression by placing it under the control of a synthetic promoter.

In certain embodiments, the one or more feedback inhibition alleviating mutations may be present in the ARO4 gene. ARO4 mutations of interest may include, but are not limited to, substitution of the lysine residue at position 229 with a leucine, a substitution of the glutamine residue at position 166 with a lysine residue, or a mutation as described by Hartmann M, et al. ((2003) Proc Natl Acad Sci USA 100(3):862-867) or Fukuda et al. ((1992) J Ferment Bioeng 74(2):117-119). In some instances, mutations for conferring feedback inhibition may be selected from a mutagenized library of enzyme mutants. Examples of such selections may include rescue of growth of o-fluoro-D,L-phenylalanine or growth of aro3 mutant yeast strains in media with excess tyrosine as described by Fukuda et al. ((1990) Breeding of Brewing Yeast Producing a Large Amount of Beta-Phenylethyl Alcohol and Beta-Phenylethyl Acetate. Agr Biol Chem Tokyo 54(1):269-271).

In certain embodiments, the engineered host cells of the present invention may include 1 or more, 2 or more, 3 or more, 4 or more, 5 or more, 6 or more, 7 or more, 8 or more, 9 or more, 10 or more, 11 or more, 12 or more, 13 or more, 14 or more, or even 15 or more feedback inhibition alleviating mutations, such as 1, 2, 3, 4, 5, 6, 7, 8, 9, 10, 11, 12, 13, 14 or 15 feedback inhibition alleviating mutations in one or more biosynthetic enzyme genes within the engineered host cell.

### Transcriptional Modulation Modifications

The host cells may include one or more transcriptional modulation modifications (such as two or more, three or more, four or more, five or more, or even more modifications) of one or more biosynthetic enzyme genes of the cell. In some examples, the one or more biosynthetic enzyme genes are native to the cell. In some examples, the one or more biosynthetic enzyme genes are non-native to the cell. Any convenient biosynthetic enzyme genes of the cell may be targeted for transcription modulation. By transcription modulation is meant that the expression of a gene of interest in a modified cell is modulated, e.g., increased or decreased, enhanced or repressed, relative to a control cell (e.g., an unmodified cell). In some cases, transcriptional modulation of the gene of interest includes increasing or enhancing expression. By increasing or enhancing expression is meant that the expression level of the gene of interest is increased by 2-fold or more, such as by 5-fold or more and sometimes by 25-, 50-, or 100-fold or more and in certain embodiments 300-fold or more or higher, as compared to a control, i.e., expression in the same cell not modified (e.g., by using any convenient gene expression assay). Alternatively, in cases where expression of the gene of interest in a cell is so low that it is undetectable, the expression level of the gene of interest is considered to be increased if expression is increased to a level that is easily detectable. In certain instances, transcriptional modulation of the gene of interest includes decreasing or repressing expression. By decreasing or repressing expression is meant that the expression level of the gene of interest is decreased by 2-fold or more, such as by 5-fold or more and sometimes by 25-, 50-, or 100-fold or more and in certain embodiments 300-fold or more or higher, as compared to a control. In some cases, expression is decreased to a level that is undetectable. Modifications of host cell processes of interest that may be adapted for use in the subject host cells are described in U.S. Publication No. 20140273109 (Ser. No. 14/211,611) by Smolke et al., the disclosure of which is herein incorporated by reference in its entirety.

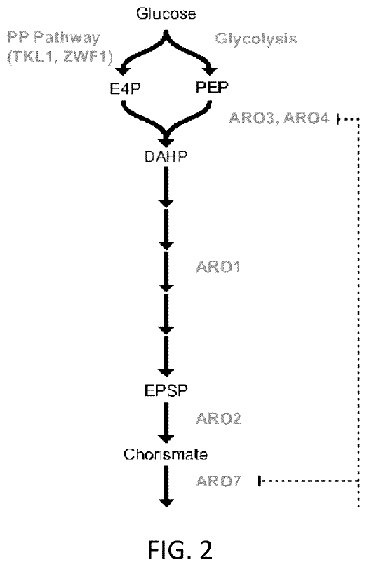

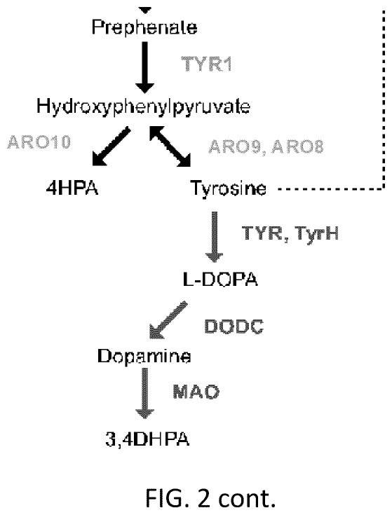

Any convenient biosynthetic enzyme genes may be transcriptionally modulated, and include but are not limited to, those biosynthetic enzymes described in FIG. 2. In particular, FIG. 2 illustrates a biosynthetic scheme for conversion of glucose to 4-HPA, dopamine, and 3,4-DHPA, in accordance with embodiments of the invention. Examples of enzymes described in FIG. 2 include ARO3, ARO4, ARO1, ARO7, TYR1, TYR, TyrH, DODC, MAO, ARO10, ARO9, ARO8, and TKL. In some instances, the one or more biosynthetic enzyme genes may be selected from ARO10, ARO9, ARO8, and TKL. In some cases, the one or more biosynthetic enzyme genes may be ARO10. In certain instances, the one or more biosynthetic enzyme genes may be ARO9. In some embodiments, the one or more biosynthetic enzyme genes may be TKL. In some embodiments, the host cell includes one or more transcriptional modulation modifications to one or more genes such as one of those genes described in Table 3.

In some embodiments, the transcriptional modulation modification may include a substitution of a strong promoter for a native promoter of the one or more biosynthetic enzyme genes or the expression of an additional copy(ies) of the gene or genes under the control of a strong promoter. The promoters driving expression of the genes of interest may be constitutive promoters or inducible promoters, provided that the promoters may be active in the host cells. The genes of interest may be expressed from their native promoters. Additionally or alternatively, the genes of interest may be expressed from non-native promoters. Although not a requirement, such promoters may be medium to high strength in the host in which they are used. Promoters may be regulated or constitutive. In some embodiments, promoters that are not glucose repressed, or repressed only mildly by the presence of glucose in the culture medium, may be used. There are numerous suitable promoters, examples of which include promoters of glycolytic genes such as the promoter of the B. subtilis tsr gene (encoding fructose biphosphate aldolase) or GAPDH promoter from yeast S. cerevisiae (coding for glyceraldehyde-phosphate dehydrogenase) (Bitter G. A., Meth. Enzymol. 152:673 684 (1987)). Other strong promoters of interest include, but are not limited to, the ADHI promoter of baker's yeast (Ruohonen L., et al, J. Biotechnol. 39:193 203 (1995)), the phosphate-starvation induced promoters such as the PHO5 promoter of yeast (Hinnen, A., et al, in Yeast Genetic Engineering, Barr, P. J., et al. eds, Butterworths (1989), the alkaline phosphatase promoter from B. lichenformis (Lee. J. W. K., et al., J. Gen. Microbiol. 137:1127 1133 (1991)), GPD1, and TEFl. Yeast promoters of interest include, but are not limited to, inducible promoters such as Gall-10, Gall, GalL, GalS, repressible promoter Met25, tetO, and constitutive promoters such as glyceraldehyde 3-phosphate dehydrogenase promoter (GPD), alcohol dehydrogenase promoter (ADH), translation-elongation factor-1-alpha promoter (TEF), cytochrome c-oxidase promoter (CYC1), MRP7 promoter, etc. In some instances, the strong promoter is GPD1. In certain instances, the strong promoter is TEFl. Autonomously replicating yeast expression vectors containing promoters inducible by hormones such as glucocorticoids, steroids, and thyroid hormones are also known and include, but are not limited to, the glucorticoid responsive element (GRE) and thyroid hormone responsive element (TRE), see e.g., those promoters described in U.S. Pat. No. 7,045,290. Vectors containing constitutive or inducible promoters such as alpha factor, alcohol oxidase, and PGH may be used. Additionally any promoter/enhancer combination (as per the Eukaryotic Promoter Data Base EPDB) could also be used to drive expression of genes of interest. It is understood that any convenient promoters specific to the host cell may be selected, e.g., E. coli. In some cases, promoter selection may be used to optimize transcription, and hence, enzyme levels to maximize production while minimizing energy resources.

### Inactivating Mutations

The engineered host cells may include one or more inactivating mutations to an enzyme of the cell (such as two or more, three or more, four or more, five or more, or even more). The inclusion of one or more inactivating mutations may modify the flux of a synthetic pathway of an engineered host cell to increase the levels of a BIA of interest or a desirable enzyme or precursor leading to the same. In some examples, the one or more inactivating mutations are to an enzyme native to the cell. Additionally or alternatively, the one or more inactivating mutations are to an enzyme non-native to the cell. As used herein, by “inactivating mutation” is meant one or more mutations to a gene or regulatory DNA sequence of the cell, where the mutation(s) inactivates a biological activity of the protein expressed by that gene of interest. In some cases, the gene is native to the cell. In some instances, the gene encodes an enzyme that is inactivated and is part of or connected to the synthetic pathway of a BIA of interest produced by the host cell. In some instances, an inactivating mutation is located in a regulatory DNA sequence that controls a gene of interest. In certain cases, the inactivating mutation is to a promoter of a gene. Any convenient mutations (e.g., as described herein) may be utilized to inactivate a gene or regulatory DNA sequence of interest. By “inactivated” or “inactivates” is meant that a biological activity of the protein expressed by the mutated gene is reduced by 10% or more, such as by 20% or more, 30% or more, 40% or more, 50% or more, 60% or more, 70% or more, 80% or more, 90% or more, 95% or more, 97% or more, or 99% or more, relative to a control protein expressed by a non-mutated control gene. In some cases, the protein is an enzyme and the inactivating mutation reduces the activity of the enzyme.

In some examples, the engineered host cell includes an inactivating mutation in an enzyme native to the cell. Any convenient enzymes may be targeted for inactivation. Enzymes of interest may include, but are not limited to those enzymes, described in Table 3 whose action in the synthetic pathway of the engineered host cell tends to reduce the levels of a BIA of interest. In some cases, the enzyme has glucose-6-phosphate dehydrogenase activity. In certain embodiments, the enzyme that includes an inactivating mutation is ZWF1. In some cases, the enzyme has alcohol dehydrogenase activity. In some embodiments, the enzyme that includes an inactivating mutation is selected from ADH2, ADH3, ADH4, ADH5, ADH6, ADH7, and SFA1. In certain embodiments, the enzyme that includes an inactivating mutation(s) is ADH2. In certain embodiments, the enzyme that includes an inactivating mutation(s) is ADH3. In certain embodiments, the enzyme that includes an inactivating mutation(s) is ADH4. In certain embodiments, the enzyme that includes an inactivating mutation(s) is ADH5. In certain embodiments, the enzyme that includes an inactivating mutation(s) is ADH6. In certain embodiments, the enzyme that includes an inactivating mutation(s) is ADH7. In some cases, the enzyme has aldehyde oxidoreductase activity. In certain embodiments, the enzyme that includes an inactivating mutation is selected from ALD2, ALD3, ALD4, ALD5, and ALD6. In certain embodiments, the enzyme that includes an inactivating mutation(s) is ALD2. In certain embodiments, the enzyme that includes an inactivating mutation(s) is ALD3. In certain embodiments, the enzyme that includes an inactivating mutation(s) is ALD4. In certain embodiments, the enzyme that includes an inactivating mutation(s) is ALD5. In certain embodiments, the enzyme that includes an inactivating mutation(s) is ALD6. In some embodiments, the host cell includes one or more inactivating mutations to one or more genes described in Table 3.

### Epimerization Modifications

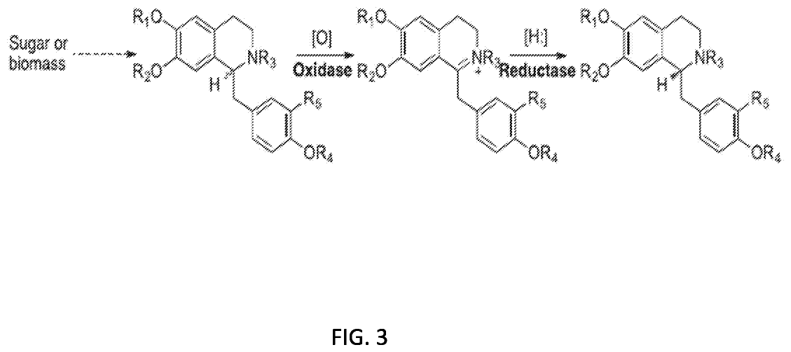

Some methods, processes, and systems provided herein describe the conversion of (S)-1-benzylisoquinoline alkaloids to (R)-1-benzylisoquinoline alkaloids. Some of these methods, processes, and systems may comprise an engineered host cell. In some examples, the conversion of (S)-1-benzylisoquinoline alkaloids to (R)-1-benzylisoquinoline alkaloids is a key step in the conversion of a substrate to a diverse range of alkaloids. In some examples, the conversion of (S)-1-benzylisoquinoline alkaloids to (R)-1-benzylisoquinoline alkaloids comprises an epimerization reaction. In some examples, the conversion of (S)-1-benzylisoquinoline alkaloids to (R)-1-benzylisoquinoline alkaloids comprises an epimerization reaction via an engineered epimerase. In some cases, epimerization of a substrate alkaloid may be performed by oxidizing an (S)-substrate to the corresponding Schiff base or imine intermediate, then stereospecifically reducing this intermediate to an (R)-product as provided in FIG. 3 and as represented generally in Scheme 1. As provided in Scheme 1, R₁, R₂, R₃, and R₄ may be H or CH₃. R₅ may be H, OH, or OCH₃.

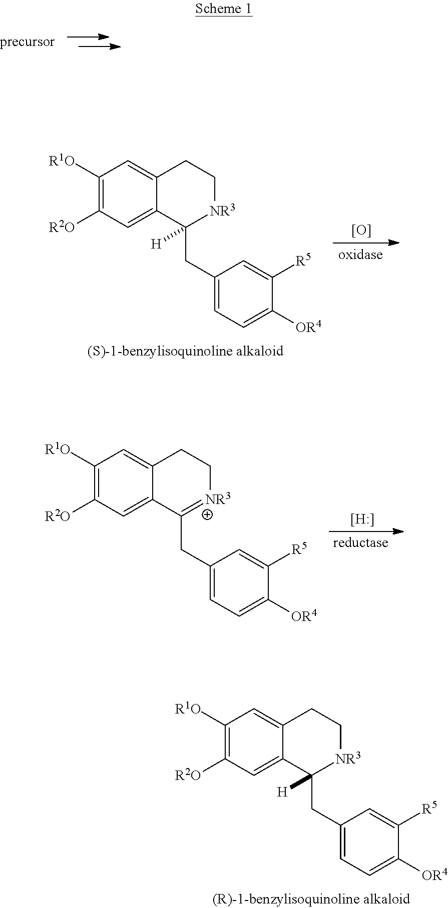

In some examples, the conversion of the (S)-substrate to the (R)-product may involve at least one oxidation reaction and at least one reduction reaction. In some cases, an oxidation reaction is optionally followed by a reduction reaction. In some cases, at least one of the oxidation and reduction reactions is carried out in the presence of an enzyme. In some cases, at least one of the oxidation and reduction reactions is catalyzed by an enzyme. In some cases, the oxidation and reduction reactions are both carried out in the presence of at least one enzyme. In some cases, at least one enzyme is useful to catalyze the oxidation and reduction reactions. The oxidation and reduction reactions may be catalyzed by the same enzyme. In some cases, at least one of the oxidation and reduction reactions is catalyzed by an engineered epimerase. In some cases, the oxidation and reduction reactions are both carried out in the presence of an engineered fused epimerase. In some cases, the oxidation and reduction reactions are both carried out in the presence of an engineered split epimerase having a separately expressed oxidase component and reductase component, respectively. In some cases, an engineered epimerase is useful to catalyze the oxidation and reduction reactions. The oxidation and reduction reactions may be catalyzed by the same engineered epimerase.

In some methods, processes and systems described herein, an oxidation reaction may be performed in the presence of an enzyme. In some examples, the enzyme may be an oxidase. In some examples, the enzyme may be part of an engineered epimerase. In some examples, the engineered epimerase may have an oxidase component. In some cases, the oxidase component may be a component of an engineered fused epimerase. In some cases, the oxidase component may be independently expressed as part of an engineered split epimerase. The oxidase may use an (S)-1-benzylisoquinoline as a substrate. The oxidase may convert the (S)-substrate to a corresponding imine or Schiff base derivative. The oxidase may be referred to as 1,2-dehydroreticuline synthase (DRS). Non-limiting examples of enzymes suitable for oxidation of (S)-1-benzylisoquinoline alkaloids in this disclosure include a cytochrome P450 oxidase, a 2-oxoglutarate-dependent oxidase, and a flavoprotein oxidase. For example, (S)-tetrahydroprotoberberine oxidase (STOX, E.C 1.3.3.8) may oxidize (S)-norreticuline and other (S)-1-benzylisoquinoline alkaloids to 1,2-dehydronorreticuline and other corresponding 1,2-dehydro products. In some examples, a protein that comprises an oxidase domain of any one of the preceding examples may perform the oxidation. In some examples, the oxidase may catalyze the oxidation reaction within a host cell, such as an engineered host cell, as described herein.

In some examples, a reduction reaction may follow the oxidation reaction. In some examples, the reduction reaction may be performed by an enzyme. In some examples, the reduction reaction may be performed by an enzyme that is part of an engineered epimerase. In some examples, the reductase may use an imine or Schiff base derived from a 1-benzylisoquinoline as a substrate. The reductase may convert the imine or Schiff base derivative to an (R)-1-benzylisoquinoline. The reductase may be referred to as 1,2-dehydroreticuline reductase (DRR). Non-limiting examples of enzymes suitable for reduction of an imine or Schiff base derived from an (S)-1-benzylisoquinoline alkaloid include an aldo-keto reductase (e.g., a codeinone reductase-like enzyme (EC 1.1.1.247)) and a short chain dehydrogenase (e.g., a salutaridine reductase-like enzyme (EC 1.1.1.248)). In some examples, a protein that comprises a reductase domain of any one of the preceding examples may perform the reduction. In a further embodiment, the reduction is stereospecific. In some examples, the reductase may catalyze the reduction reaction within a host cell, such as an engineered host cell, as described herein.

An example of an enzyme that can perform an epimerization reaction that converts (S)-1-benzylisoquinoline alkaloids to (R)-1-benzylisoquinoline alkaloids includes an epimerase having an oxidase domain and a reductase domain. In particular, the epimerase may have a cytochrome P450 oxidase 82Y2-like domain. Additionally, the epimerase may have a codeinone reductase-like domain. Further, an epimerase having a cytochrome P450 oxidase 82Y2-like domain and also having a codeinone reductase-like domain may be referred to as a DRS-DRR enzyme. In particular, a DRS-DRR enzyme may be a fusion enzyme that is a fusion epimerase. Further, when a DRS-DRR enzyme is modified by at least one activity-increasing modification, the fusion enzyme may be an engineered fusion epimerase.

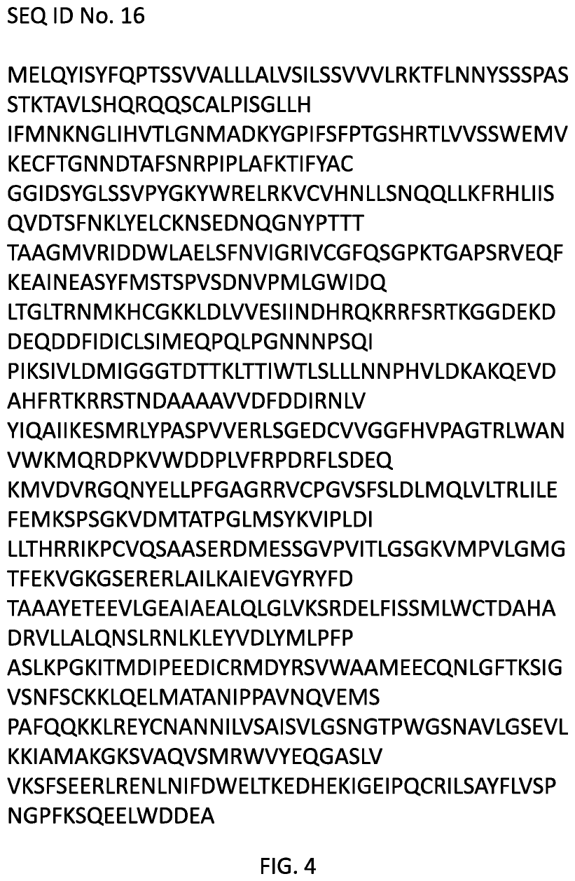

An example of an amino acid sequence of a DRS-DRR enzyme that may be used to perform the conversion of (S)-1-benzylisoquinoline alkaloids to (R)-1-benzylisoquinoline alkaloids is provided in FIG. 4. In particular, FIG. 4 illustrates an amino acid sequence of a DRS-DRR enzyme, in accordance with embodiments of the invention. As seen in FIG. 4, underlined text denotes the cytochrome P450 CYP82Y2-like domain (59% identity to AFB74617.1). The dotted underlined text denotes the aldo-keto reductase NADPH-dependent codeinone reductase-like domain (75% identity to ACM44066.1). Additional amino acid sequences of a DRS-DRR enzyme are set forth in Table 1. An amino acid sequence for an epimerase that is utilized in converting an (S)-1-benzylisoquinoline alkaloid to an (R)-1-benzylisoquinoline alkaloid may be 75% or more identical to a given amino acid sequence as listed in Table 1. For example, an amino acid sequence for such an epimerase may comprise an amino acid sequence that is at least 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the DNA sequence is altered such as to optimize codon usage for the host organism, for example.

An engineered host cell may be provided that produces an epimerase that converts (S)-1-benzylisoquinoline alkaloid to (R)-1-benzylisoquinoline alkaloid, wherein the epimerase comprises an amino acid sequence selected from the group consisting of: SEQ ID NOs: 1, 2, 3, 4, 5, 6, 7, 8, 9, 10, 11, 12, 13, 14, and 15. The epimerase that is produced within the engineered host cell may be recovered and purified so as to form a biocatalyst. In some cases, the epimerase may be split into one or more enzymes. Additionally, one or more enzymes that are produced by splitting the epimerase may be recovered from the engineered host cell. These one or more enzymes that result from splitting the epimerase may also be used to catalyze the conversion of (S)-1-benzylisoquinoline alkaloids to (R)-1-benzylisoquinoline alkaloids. In particular, the one or more enzymes that are recovered from the engineered host cell that produces the epimerase may be used in a process for converting an (S)-1-benzylisoquinoline alkaloid to an (R)-1-benzylisoquinoline alkaloid. The process may include contacting the (S)-1-benzylisoquinoline alkaloid with an epimerase in an amount sufficient to convert said (S)-1-benzylisoquinoline alkaloid to (R)-1-benzylisoquinoline alkaloid. In examples, the (S)-1-benzylisoquinoline alkaloid may be contacted with a sufficient amount of the one or more enzymes such that at least 5% of said (S)-1-benzylisoquinoline alkaloid is converted to (R)-1-benzylisoquinoline alkaloid. In further examples, the (S)-1-benzylisoquinoline alkaloid may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said (S)-1-benzylisoquinoline alkaloid is converted to (R)-1-benzylisoquinoline alkaloid.

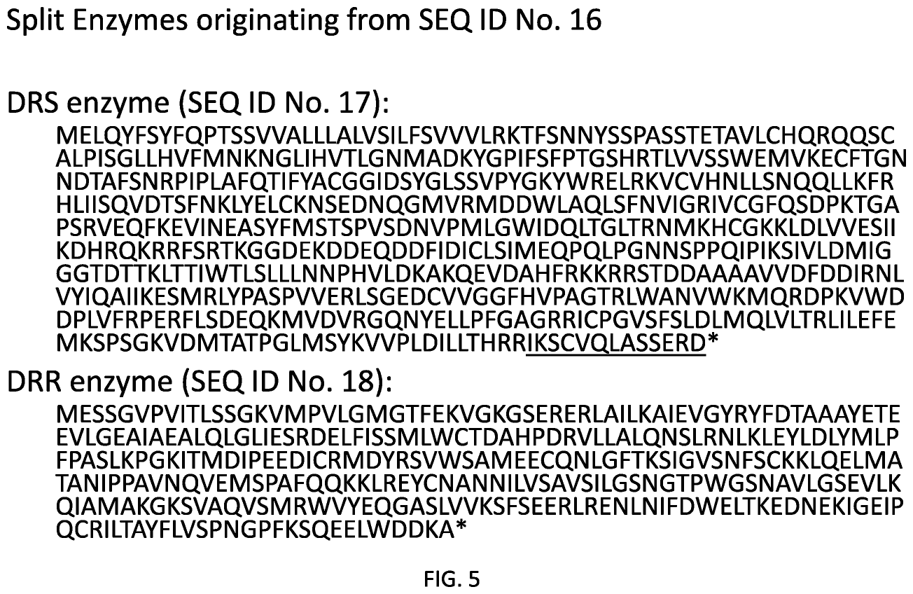

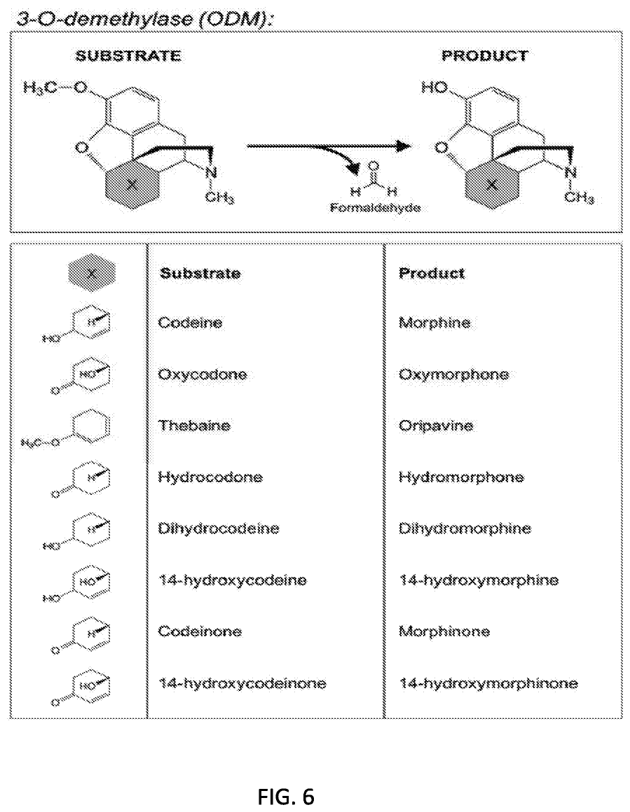

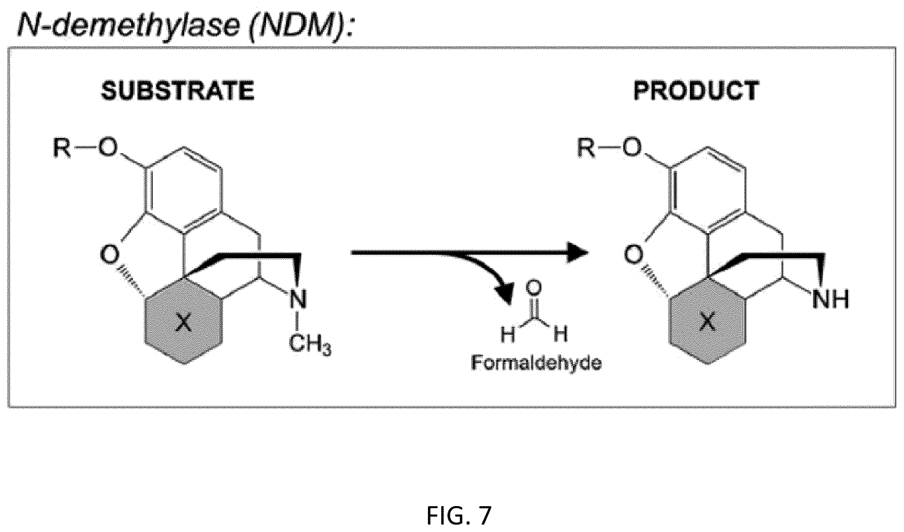

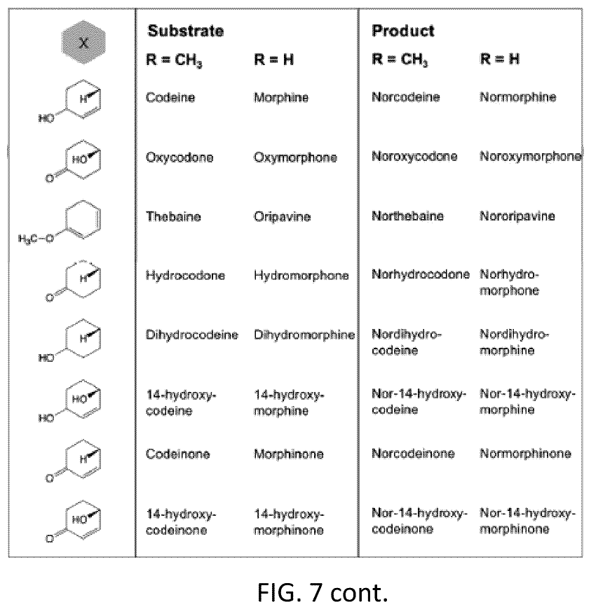

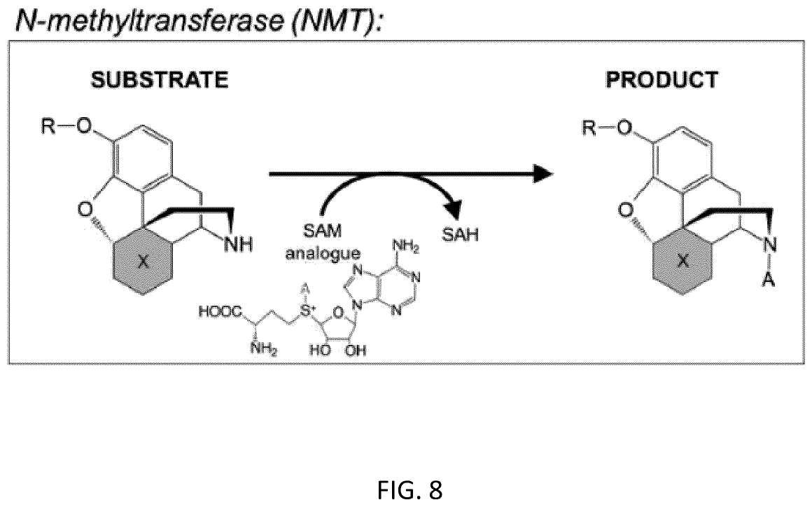

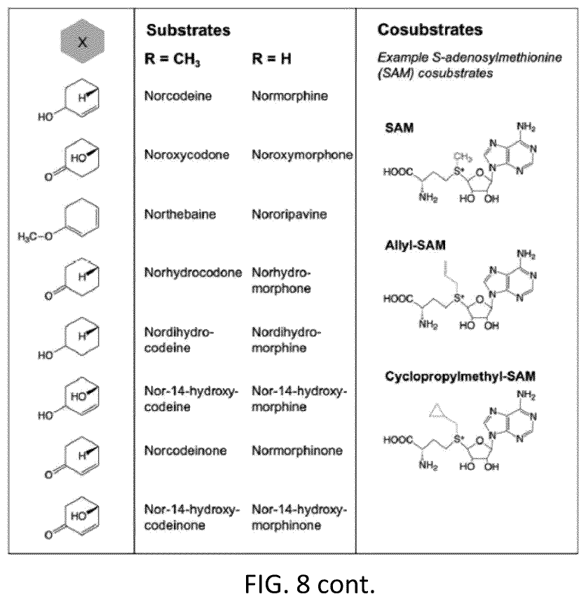

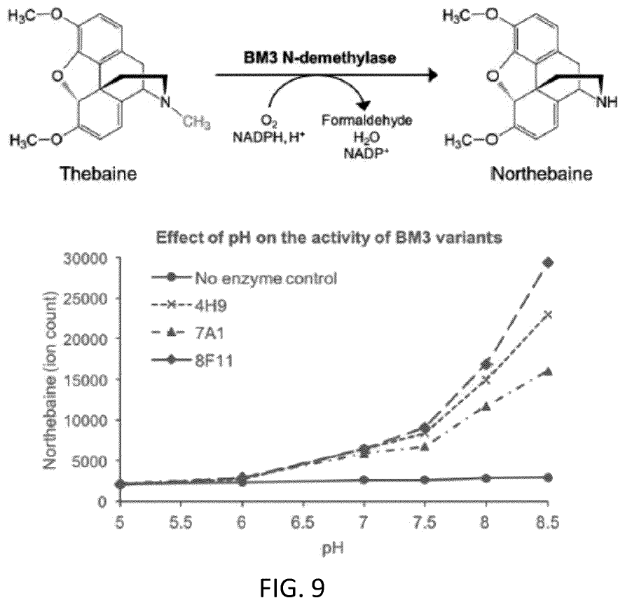

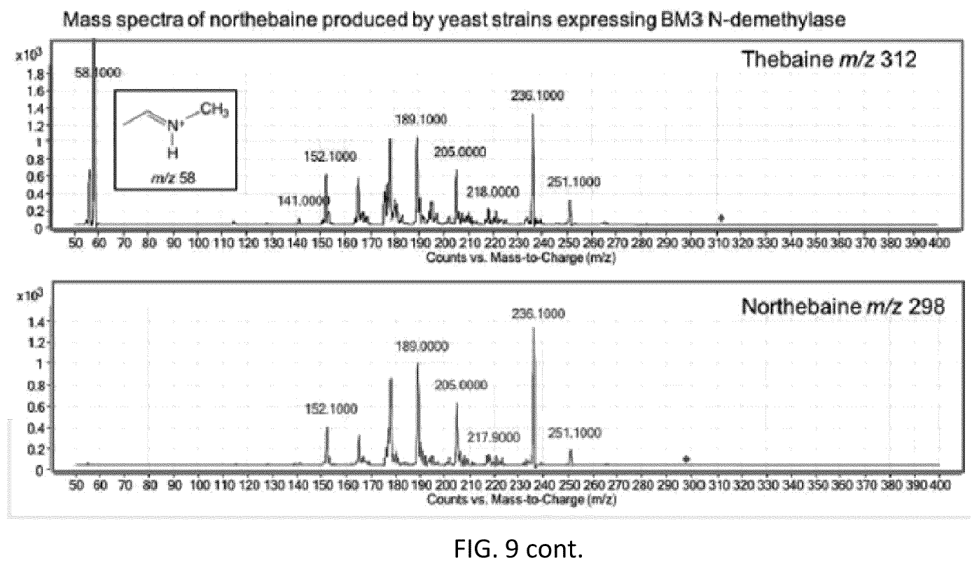

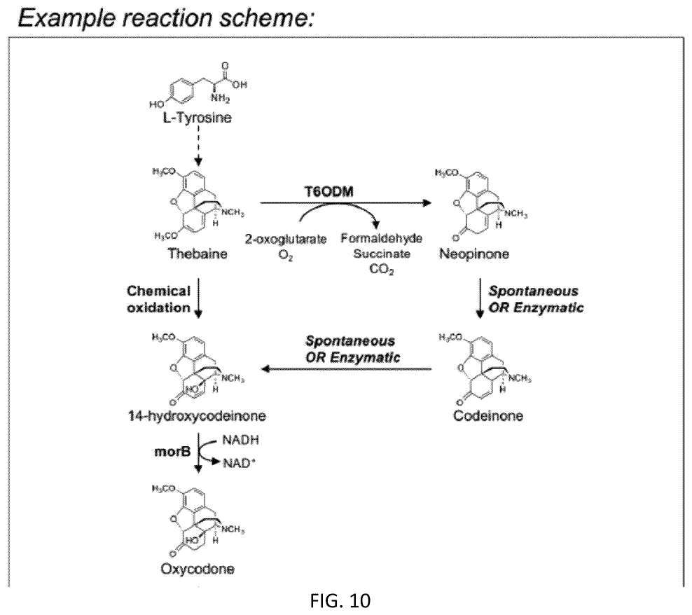

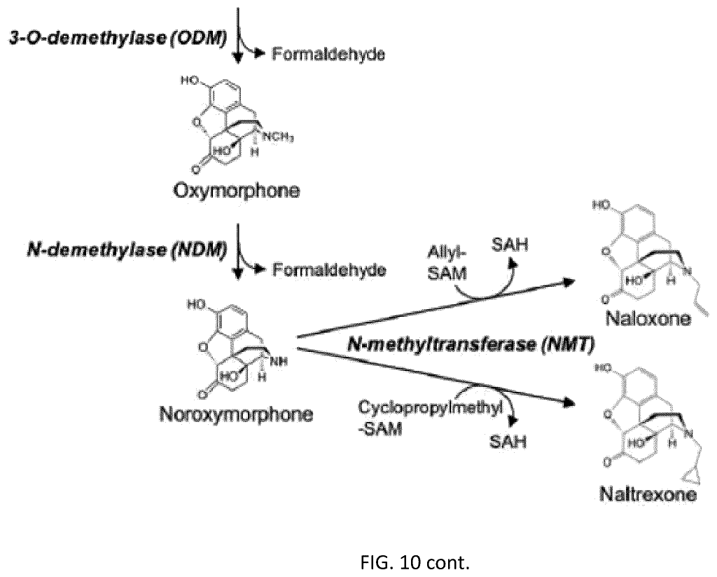

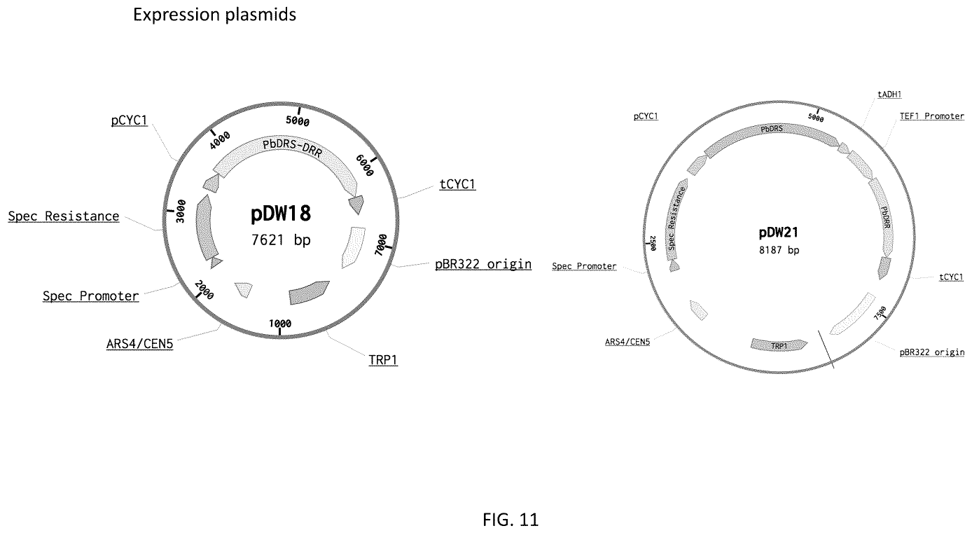

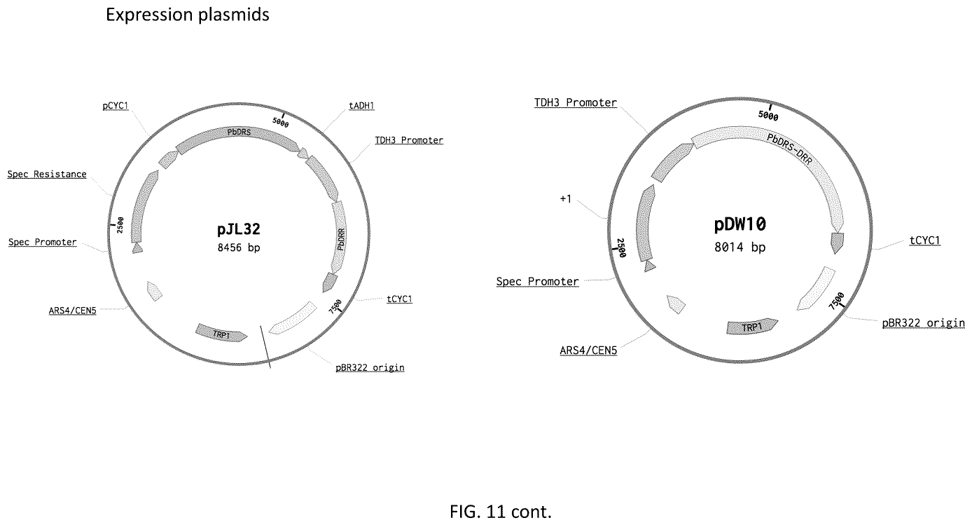

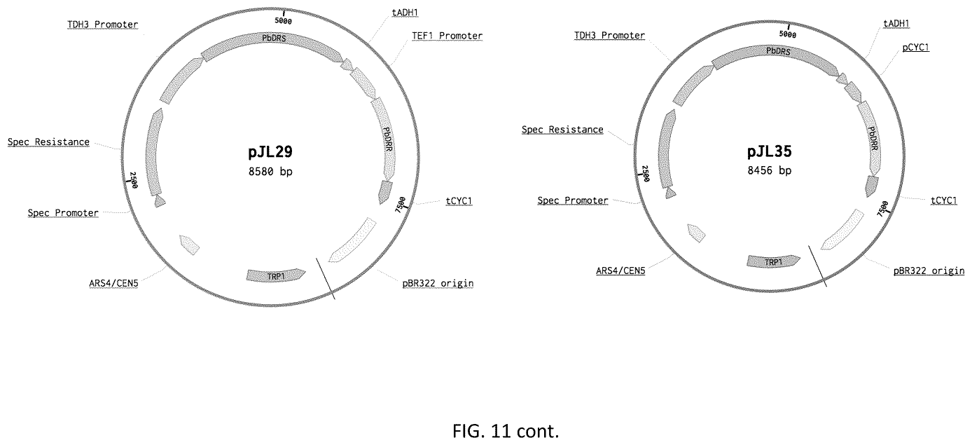

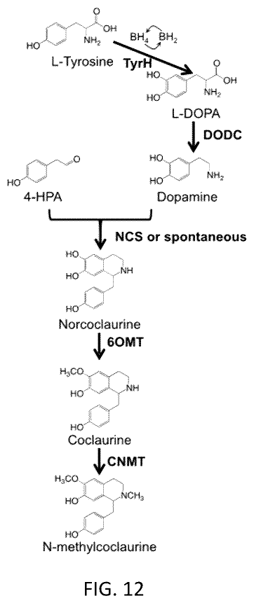

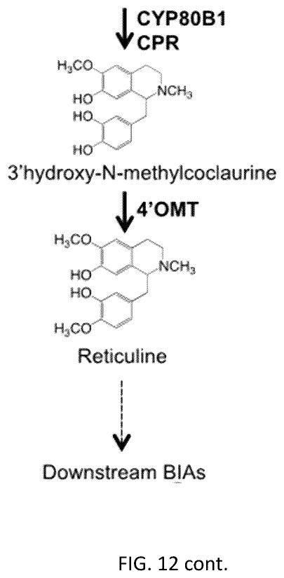

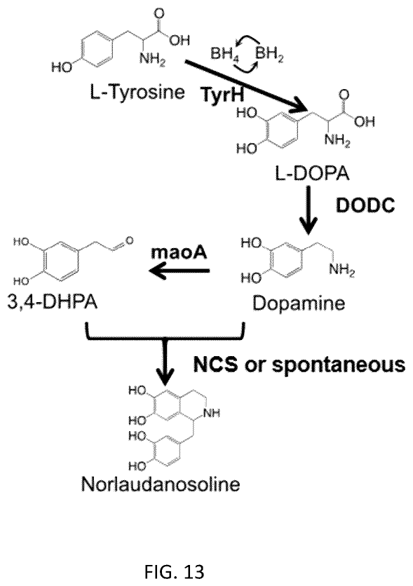

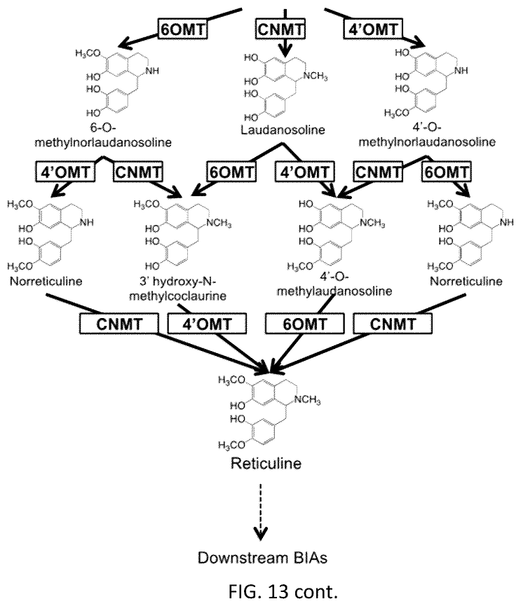

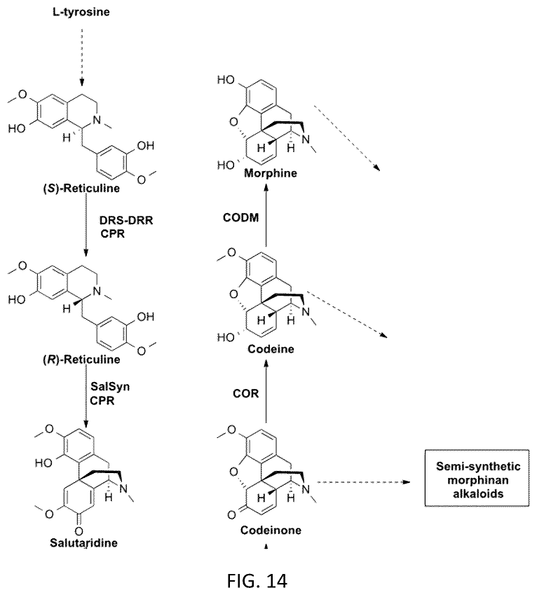

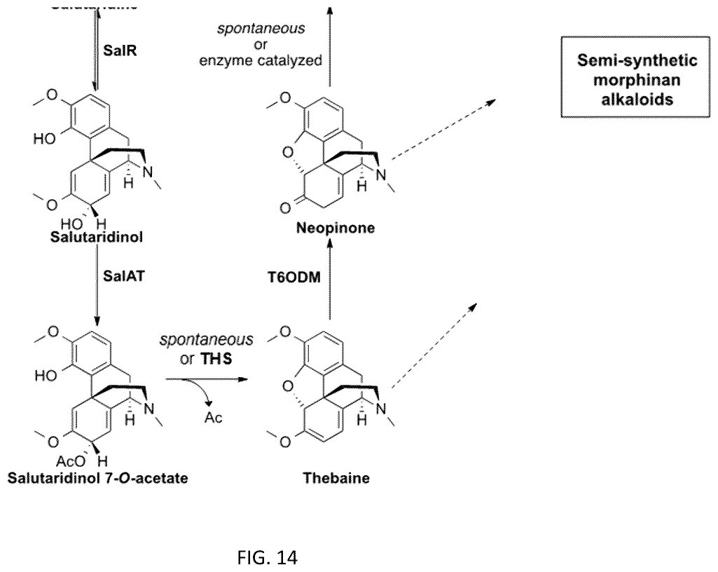

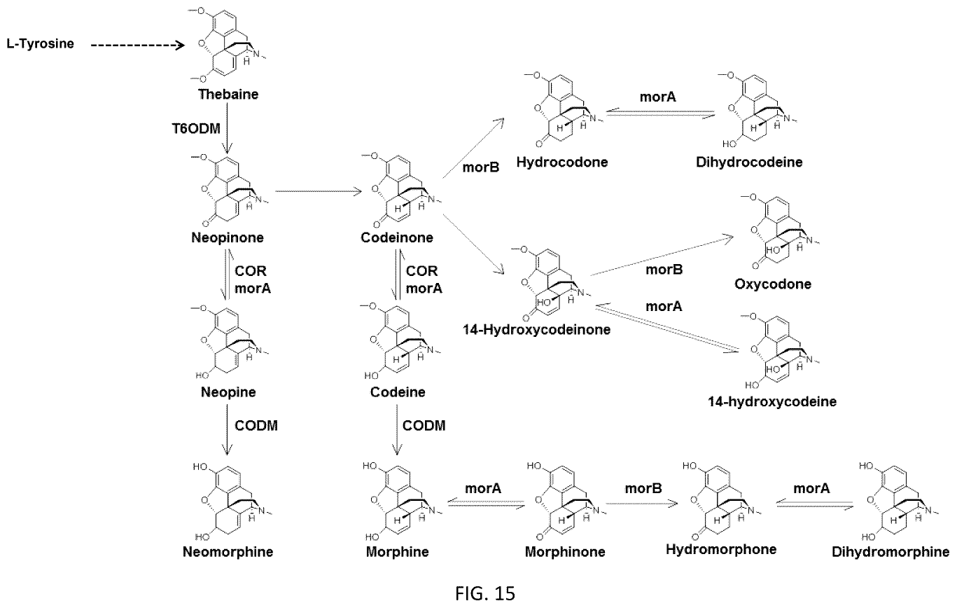

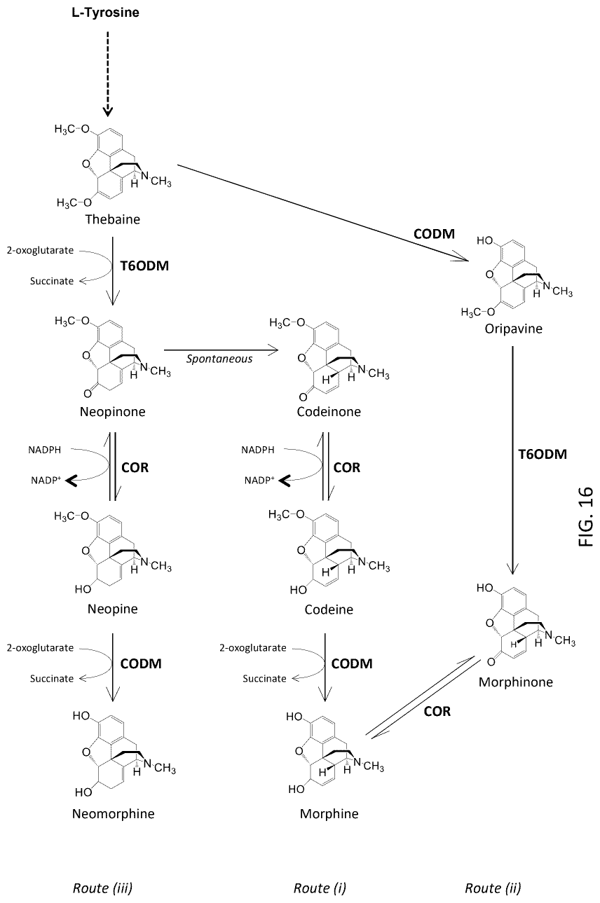

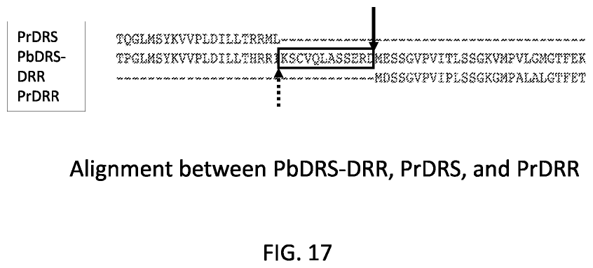

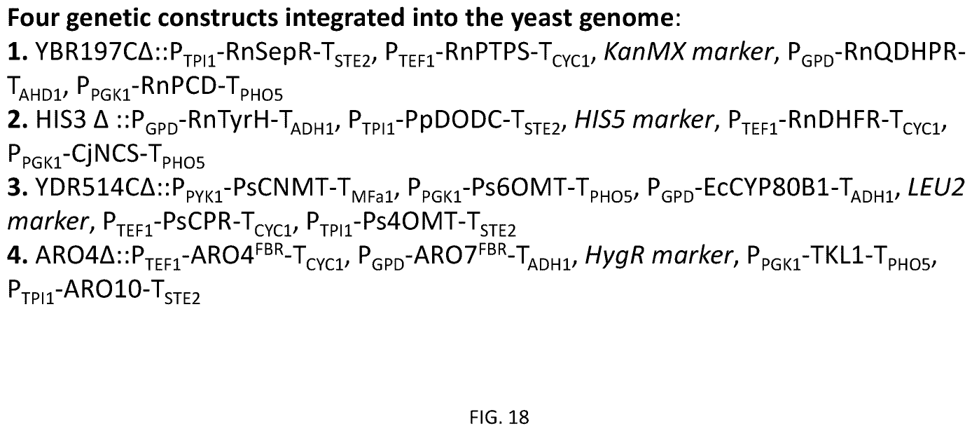

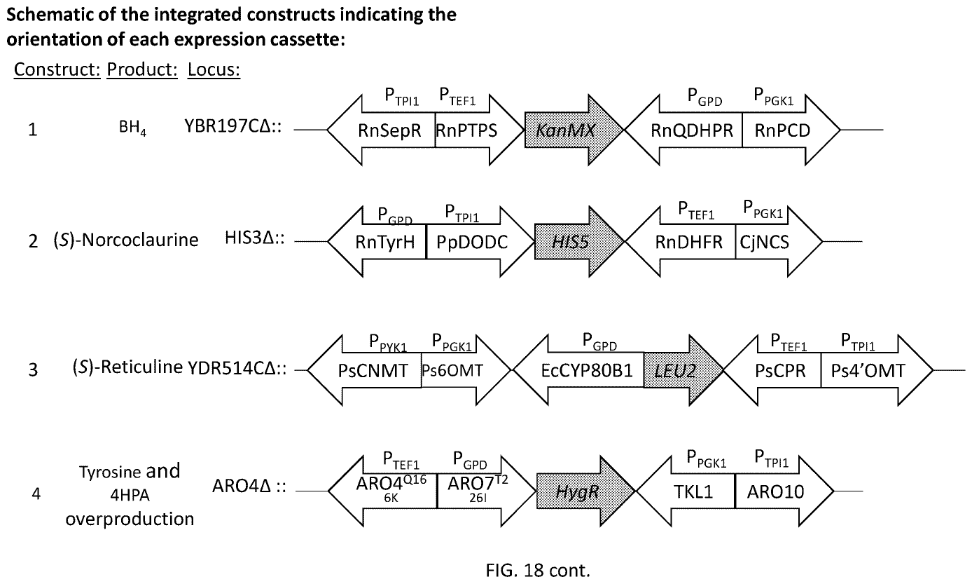

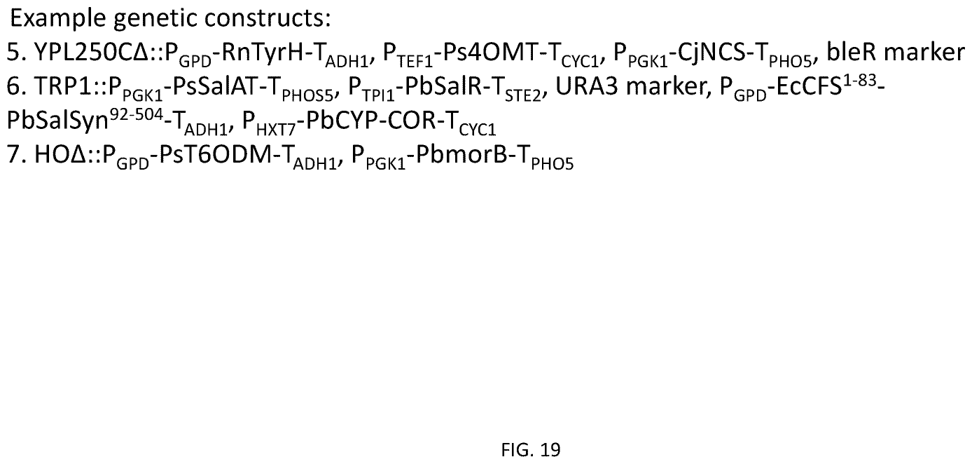

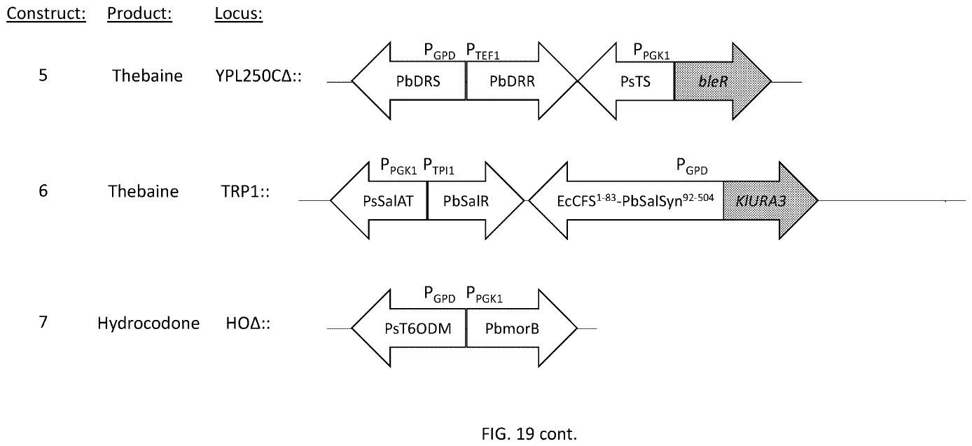

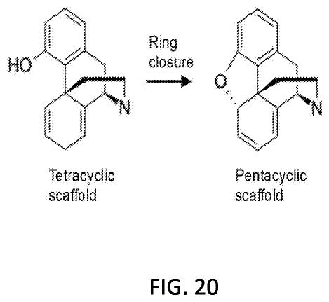

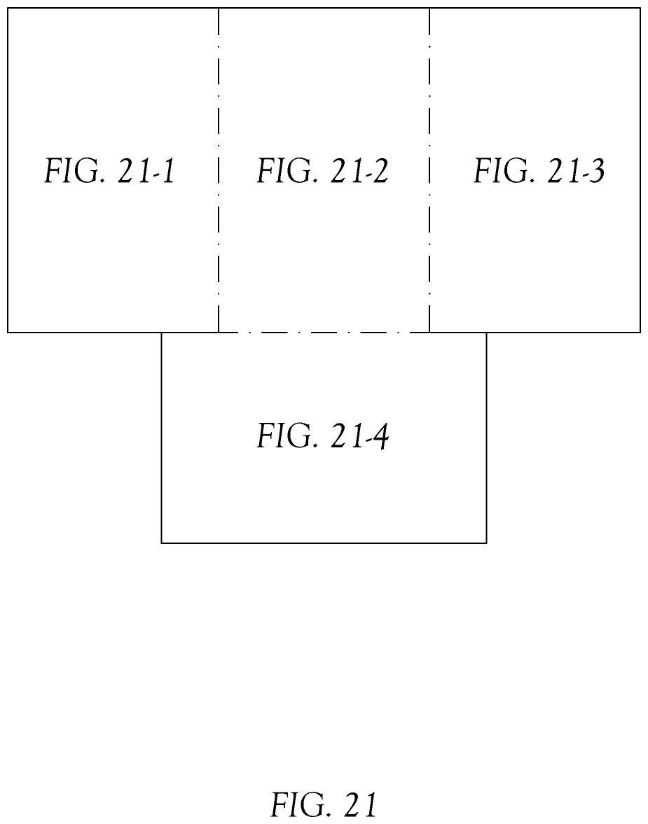

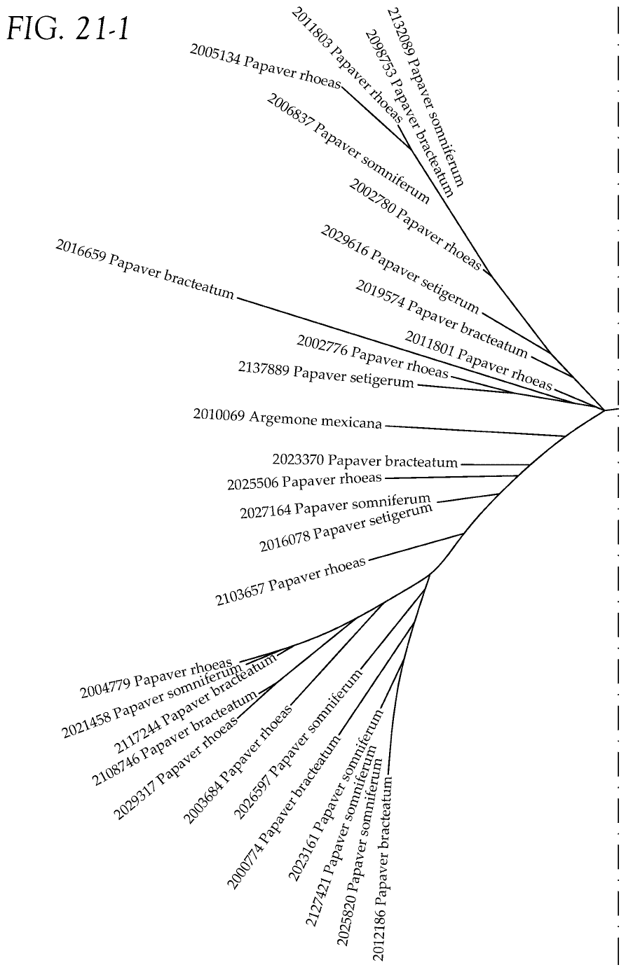

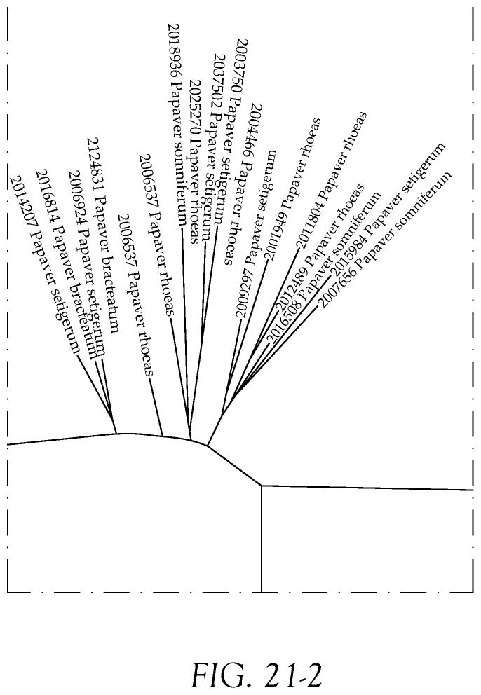

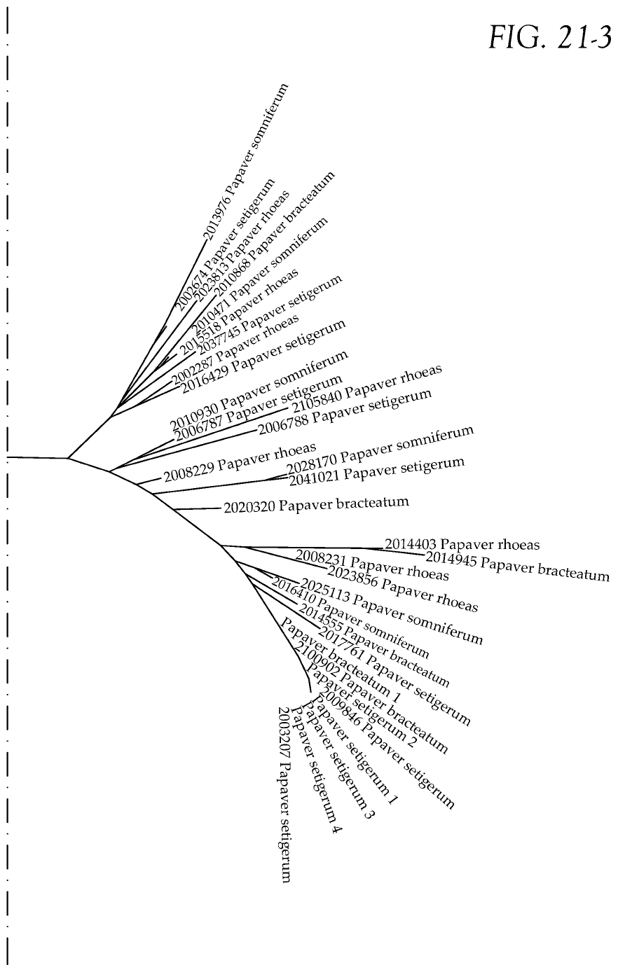

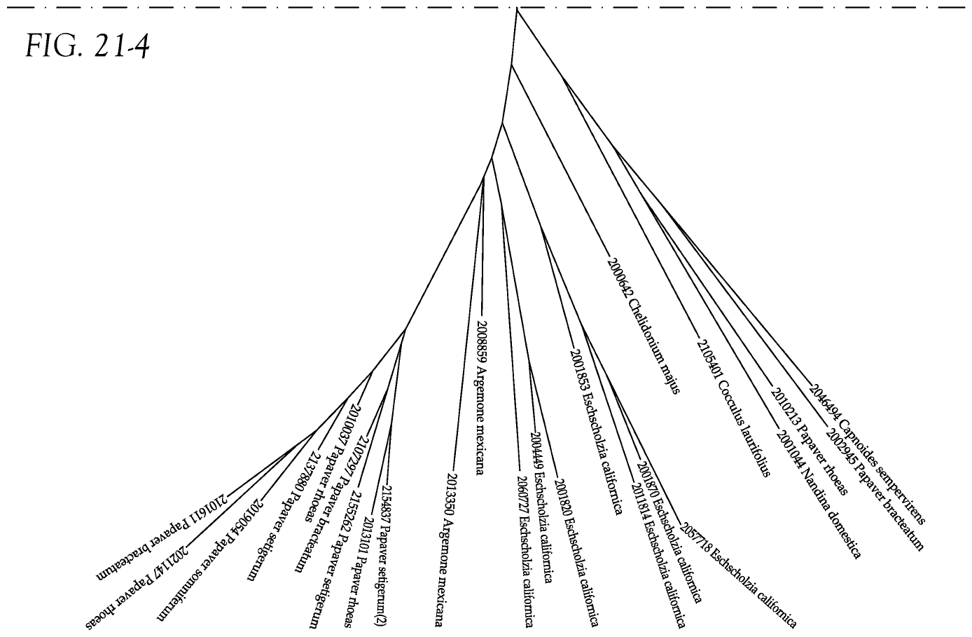

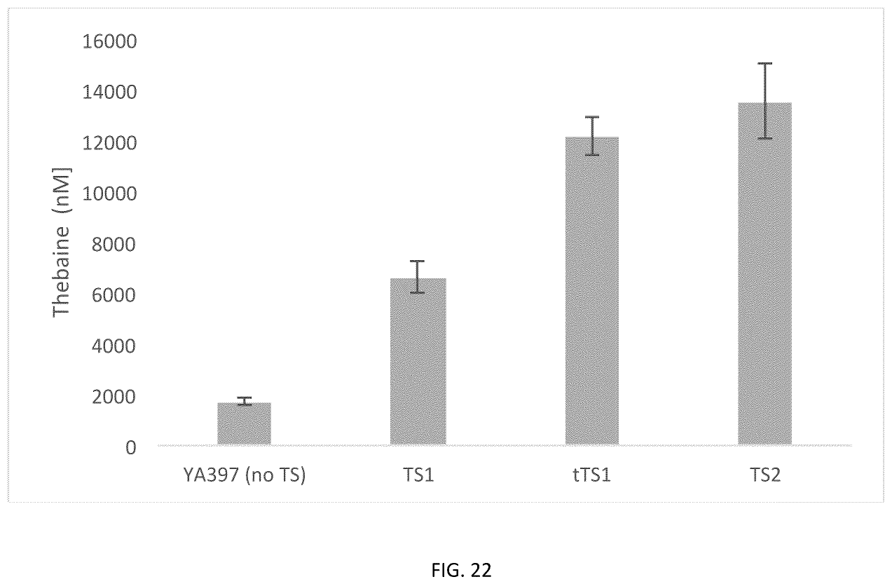

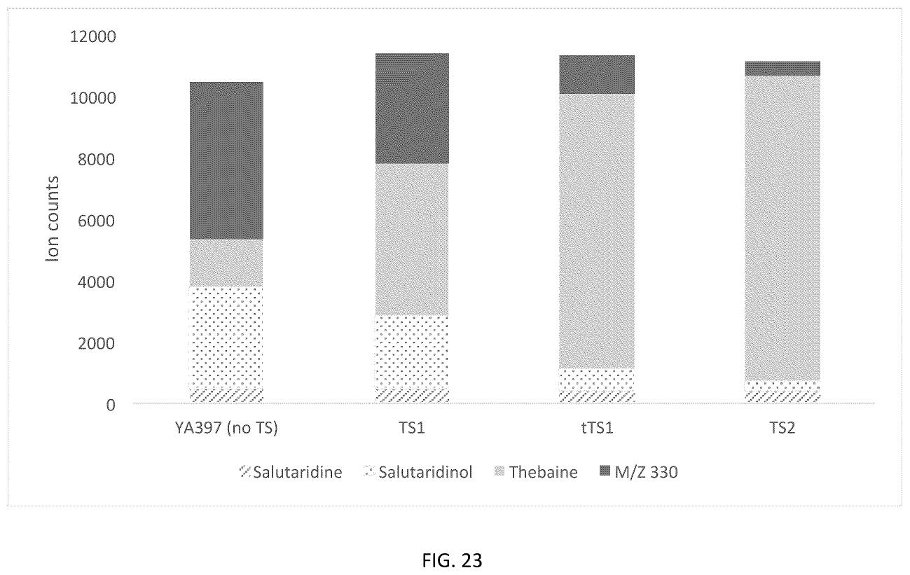

An example of an amino acid sequence of a DRS-DRR enzyme that may be used to perform the conversion of (S)-1-benzylisoquinoline alkaloids to (R)-1-benzylisoquinoline alkaloids is provided in FIG. 4. In particular, FIG. 4 illustrates an amino acid sequence of a DRS-DRR enzyme that has been codon-optimized, in accordance with embodiments of the invention. Further, FIG. 5 illustrates a split of an oxidase portion and reductase portion, each of the DRS-DRR enzyme of FIG. 4. Additional amino acid sequences of a DRS-DRR enzyme are set forth in Table 1. An amino acid sequence for an epimerase that is utilized in converting an (S)-1-benzylisoquinoline alkaloid to an (R)-1-benzylisoquinoline alkaloid may be 75% or more identical to a given amino acid sequence as listed in Table 1. For example, an amino acid sequence for such an epimerase may comprise an amino acid sequence that is at least 75% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to an amino acid sequence as provided herein. Additionally, in certain embodiments, an “identical” amino acid sequence contains at least 80%-99% identity at the amino acid level to the specific amino acid sequence. In some cases an “identical” amino acid sequence contains at least about 85%, 86%, 87%, 88%, 89%, 90%, 91%, 92%, 93%, 94% and more in certain cases, at least 95%, 96%, 97%, 98% and 99% identity, at the amino acid level. In some cases, the amino acid sequence may be identical but the DNA sequence is altered such as to optimize codon usage for the host organism, for example.

Amino acid residues of homologous epimerases may be referenced according to the numbering scheme of SEQ ID NO. 16, and this numbering system is used throughout the disclosure to refer to specific amino acid residues of epimerases which are homologous to SEQ ID NO. 16. Epimerases homologous to SEQ ID NO. 16 may have at least about 70%, 75%, 80%, 85%, 90%, 95%, 97%, 98%, or 99% sequence identity to SEQ ID NO. 16. In some cases, an amino acid referred to as position 50 in a homologous epimerase may not be the 50^{th} amino acid in the homologous epimerase, but would be the amino acid which corresponds to the amino acid at position 50 in SEQ ID NO. 16 in a protein alignment of the homologous epimerase with SEQ ID NO. 16. In some cases, homologous enzymes may be aligned with SEQ ID NO. 16 either according to primary sequence, secondary structure, or tertiary structure.

An engineered host cell may be provided that produces an engineered epimerase that converts (S)-1-benzylisoquinoline alkaloid to (R)-1-benzylisoquinoline alkaloid, wherein the epimerase comprises an amino acid sequence selected from the group consisting of: SEQ ID NOs: 1, 2, 3, 4, 5, 6, 7, 8, 9, 10, 11, 12, 13, 14, 15, 16, 17, and 18, and having one or more activity-enhancing modifications. The epimerase that is produced within the engineered host cell may be recovered and purified so as to form a biocatalyst. In some cases, the epimerase may be split into one or more enzymes. Additionally, one or more enzymes that are produced by splitting the epimerase may be recovered from the engineered host cell. These one or more enzymes that result from splitting the epimerase may also be used to catalyze the conversion of (S)-1-benzylisoquinoline alkaloids to (R)-1-benzylisoquinoline alkaloids. Additionally, the use of an engineered split epimerase may be used to increase the production of benzylisoquinoline alkaloid products within a cell when compared to the production of benzylisoquinoline alkaloid products within a cell utilizing a fused epimerase.

In additional cases, the one or more enzymes that are recovered from the engineered host cell that produces the epimerase may be used in a process for converting an (S)-1-benzylisoquinoline alkaloid to an (R)-1-benzylisoquinoline alkaloid. The process may include contacting the (S)-1-benzylisoquinoline alkaloid with an epimerase in an amount sufficient to convert said (S)-1-benzylisoquinoline alkaloid to (R)-1-benzylisoquinoline alkaloid. In examples, the (S)-1-benzylisoquinoline alkaloid may be contacted with a sufficient amount of the one or more enzymes such that at least 5% of said (S)-1-benzylisoquinoline alkaloid is converted to (R)-1-benzylisoquinoline alkaloid. In further examples, the (S)-1-benzylisoquinoline alkaloid may be contacted with a sufficient amount of the one or more enzymes such that at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100% of said (S)-1-benzylisoquinoline alkaloid is converted to (R)-1-benzylisoquinoline alkaloid.

The one or more enzymes that may be used to convert an (S)-1-benzylisoquinoline alkaloid to an (R)-1-benzylisoquinoline alkaloid may contact the (S)-1-benzylisoquinoline alkaloid in vitro. Additionally, or alternatively, the one or more enzymes that may be used to convert an (S)-1-benzylisoquinoline alkaloid to an (R)-1-benzylisoquinoline alkaloid may contact the (S)-1-benzylisoquinoline alkaloid in vivo. Additionally, the one or more enzymes that may be used to convert an (S)-1-benzylisoquinoline alkaloid to an (R)-1-benzylisoquinoline alkaloid may be provided to a cell having the (S)-1-benzylisoquinoline alkaloid within, or may be produced within an engineered host cell.

In some examples, the methods provide for engineered host cells that produce an alkaloid product, wherein the epimerization of an (S)-substrate to an (R)-product may comprise a key step in the production of an alkaloid product. In some examples, the alkaloid produced is an (R)-1-benzylisoquinoline alkaloid. In still other embodiments, the alkaloid produced is derived from an (R)-1-benzylisoquinoline alkaloid, including, for example, 4-ring promorphinan and 5-ring morphinan alkaloids. In another embodiment, an (S)-1-benzylisoquinoline alkaloid is an intermediate toward the product of the engineered host cell. In still other embodiments, the alkaloid product is selected from the group consisting of 1-benzylisoquinoline, morphinan, promorphinan, nor-opioid, or nal-opioid alkaloids.

In some examples, the (S)-substrate is an (S)-1-benzylisoquinoline alkaloid selected from the group consisting of (S)-norreticuline, (S)-reticuline, (S)-tetrahydropapaverine, (S)-norcoclaurine, (S)-coclaurine, (S)—N-methylcoclaurine, (S)-3′-hydroxy-N-methylcoclaurine, (S)-norisoorientaline, (S)-orientaline, (S)-isoorientaline, (S)-norprotosinomenine, (S)-protosinomenine, (S)-norlaudanosoline, (S)-laudanosoline, (S)-4′-O-methyllaudanosoline, (S)-6-O-methylnorlaudanosoline, (S)-4′-O-methylnorlaudanosoline.

In some examples, the (S)-substrate is a compound of Formula I:

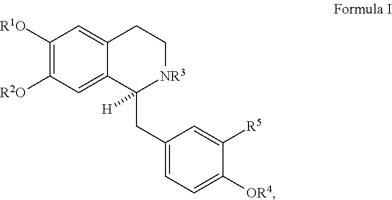

or a salt thereof, wherein:
R¹, R², R³, and R⁴ are independently selected from hydrogen and methyl; and
R⁵ is selected from hydrogen, hydroxy, and methoxy.

In some other examples, at least one of R¹, R², R³, R⁴, and R⁵ is hydrogen.

In still other examples, the (S)-substrate is a compound of Formula II:

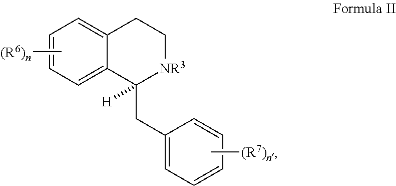

or a salt thereof, wherein:
R³ is selected from hydrogen and C₁-C₄ alkyl;
R⁶ and R⁷ are independently selected at each occurrence from hydroxy, fluoro, chloro, bromo, carboxaldehyde, C1-C₄ acyl, C1-C₄ alkyl, and C1-C₄ alkoxy;
n is 0, 1, 2, 3, or 4; and
n′ is 0, 1, 2, 3, 4 or 5.

When a bond is drawn across a ring, it means substitution may occur at a non-specific ring atom or position. For example, in Formula II shown above, the hydrogen of any —CH— in the 6-membered ring may be replaced with R⁷ to form —CR⁷—.

In some examples, R⁶ and R⁷ are independently methyl or methoxy. In some other examples, n and n′ are independently 1 or 2. In still other embodiments, R³ is hydrogen or methyl.

In some examples, the methods provide for engineered host cells that produce alkaloid products from (S)-reticuline. The epimerization of (S)-reticuline to (R)-reticuline may comprise a key step in the production of diverse alkaloid products from a precursor. In some examples, the precursor is L-tyrosine or a sugar (e.g., glucose). The diverse alkaloid products can include, without limitation, 1-benzylisoquinoline, morphinan, promorphinan, nor-opioid, or nal-opioid.alkaloids.

Any suitable carbon source may be used as a precursor toward an epimerized 1-benzylisoquinoline alkaloid. Suitable precursors can include, without limitation, monosaccharides (e.g., glucose, fructose, galactose, xylose), oligosaccharides (e.g., lactose, sucrose, raffinose), polysaccharides (e.g., starch, cellulose), or a combination thereof. In some examples, unpurified mixtures from renewable feedstocks can be used (e.g., cornsteep liquor, sugar beet molasses, barley malt, biomass hydrolysate). In still other embodiments, the carbon precursor can be a one-carbon compound (e.g., methanol, carbon dioxide) or a two-carbon compound (e.g., ethanol). In yet other embodiments, other carbon-containing compounds can be utilized, for example, methylamine, glucosamine, and amino acids (e.g., L-tyrosine). In some examples, a 1-benzylisoquinoline alkaloid may be added directly to an engineered host cell of the invention, including, for example, norlaudanosoline, laudanosoline, norreticuline, and reticuline. In still further embodiments, a 1-benzylisoquinoline alkaloid may be added to the engineered host cell as a single enantiomer (e.g., an (S)-1-benzylisoquinoline alkaloid), or a mixture of enantiomers, including, for example, a racemic mixture.

In some examples, the methods provide for the epimerization of a stereocenter of a 1-benzylisoquinoline alkaloid, or a derivative thereof. In a further embodiment, the method comprises contacting the 1-benzylisoquinoline alkaloid with at least one enzyme. The at least one enzyme may invert the stereochemistry of a stereocenter of a 1-benzylisoquinoline alkaloid, or derivative thereof, to the opposite stereochemistry. In some examples, the at least one enzyme converts an (S)-1-benzylisoquinoline alkaloid to an (R)-1-benzylisoquinoline alkaloid. In some examples of this conversion of an (S)-1-benzylisoquinoline alkaloid to an (R)-1-benzylisoquinoline alkaloid utilizing the at least one enzyme, the (S)-1-benzylisoquinoline alkaloid is selected from the group consisting of (S)-norreticuline, (S)-reticuline, (S)-tetrahydropapaverine, (S)-norcoclaurine, (S)-coclaurine, (S)—N-methylcoclaurine, (S)-3′-hydroxy-N-methylcoclaurine, (S)-norisoorientaline, (S)-orientaline, (S)-isoorientaline, (S)-norprotosinomenine, (S)-protosinomenine, (S)-norlaudanosoline, (S)-laudanosoline, (S)-4′-O-methyllaudanosoline, (S)-6-O-methylnorlaudanosoline, and (S)-4′-O-methylnorlaudanosoline.

In still other embodiments, the 1-benzylisoquinoline alkaloid that is epimerized may comprise two or more stereocenters, wherein only one of the two or more stereocenters is inverted to produce a diastereomer of the substrate (e.g., (S, R)-1-benzylisoquinoline alkaloid converted to (R, R)-1-benzylisoquinoline alkaloid). In examples where only one stereocenter of a 1-benzylisoquinoline alkaloid is inverted when contacted with the at least one enzyme, the product is referred to as an epimer of the 1-benzylisoquinoline alkaloid.

In some examples, the 1-benzylisoquinoline alkaloid is presented to the enzyme as a single stereoisomer. In some other examples, the 1-benzylisoquinoline alkaloid is presented to the enzyme as a mixture of stereoisomers. In still further embodiments, the mixture of stereoisomers may be a racemic mixture. In some other examples, the mixture of stereoisomers may be enriched in one stereoisomer as compared to another stereoisomer.

In some examples, an 1-benzylisoquinoline alkaloid, or a derivative thereof, is recovered. In some examples, the 1-benzylisoquinoline alkaloid is recovered from a cell culture. In still further embodiments, the recovered 1-benzylisoquinoline alkaloid is enantiomerically enriched in one stereoisomer as compared to the original mixture of 1-benzylisoquinoline alkaloids presented to the enzyme. In still further embodiments, the recovered 1-benzylisoquinoline alkaloid has an enantiomeric excess of at least 50%, at least 55%, at least 60%, at least 65%, at least 70%, at least 75%, at least 80%, at least 82%, at least 84%, at least 86%, at least 88%, at least 90%, at least 91%, at least 92%, at least 93%, at least 94%, at least 95%, at least 96%, at least 97%, at least 98%, at least 99%, at least 99.5%, at least 99.7%, or 100%.

In some examples, a promorphinan, or a derivative thereof, is recovered. In some examples, the promorphinan is recovered from a cell culture.

In some examples, a morphinan, or a derivative thereof, is recovered. In some examples, the morphinan is recovered from a cell culture.

In some examples, a nal-opioid, or a derivative thereof, is recovered. In some examples, the nal-opioid is recovered from a cell culture.

In some examples, a nor-opioid, or a derivative thereof, is recovered. In some examples, the nor-opioid is recovered from a cell culture.

“Isomers” are different compounds that have the same molecular formula. “Stereoisomers” are isomers that differ only in the way the atoms are arranged in space. “Enantiomers” are a pair of stereoisomers that are non superimposable mirror images of each other. A 1:1 mixture of a pair of enantiomers is a “racemic” mixture. “Diastereoisomers” or “diastereomers” are stereoisomers that have at least two asymmetric atoms but are not mirror

1-73. (canceled)

74. A method of converting a precursor of a promorphinan molecule to an alkaloid product comprising:
contacting the precursor of a promorphinan molecule with an epimerase and a thebaine synthase, wherein at least one promorphinan precursor is produced within an engineered non-plant cell where the conversion occurs,
wherein the engineered non plant cell comprises three or more enzymes selected from the group consisting of tyrosinase or tyrosine hydroxylase (TYR or Tyr1), L DOPA decarboxylase (DODC), monoamine oxidase (maoA), 6 O methyltransferase (60MT), coclaurine N methyltransferase (CNMT), cytochrome P450 80B1 (CYP80B1), 4′ 0 methyltransferase (4′OMT), dehydroreticuline synthase and dehydroreticuline reductase (DRS DRR), salutaridine synthase (SalSyn), salutaridine reductase (SalR), salutaridinol 7 O acetyltransferase (SalAT), and norcoclaurine synthase (NCS);
and wherein, within the engineered non plant cell, the precursor of a promorphinan molecule or is converted to an alkaloid product selected from the group consisting of (i) a morphinan alkaloid, (ii) a nal opioid alkaloid, and (iii) a nor opioid alkaloid.

75. The method of claim 74, wherein the alkaloid product is a morphinan alkaloid.

76. The method of claim 75, wherein the morphinan alkaloid is a thebaine, codeinone, codeine, morphine, morphinone, oripavine, neopinone, neopine, neomorphine, hydrocodone, dihydrocodeine, 14-hydroxycodeinone, oxycodone, 14-hydroxycodeine, morphinone, hydromorphone, dihydromorphine, dihydroetorphine, ethylmorphine, etorphine, metopon, buprenorphine, pholcodine, heterocodeine, or oxymorphone.

77. The method of claim 74, wherein the alkaloid product is a nal opioid alkaloid.

78. The method of claim 77, wherein the nal opioid alkaloid is a naltrexone, naloxone, nalmefene, nalorphine, nalorphine, nalodeine, naldemedine, naloxegol, 6b-naltrexol, naltrindole, methylnaltrexone, methylsamidorphan, alvimopan, axelopran, bevenpran, dinicotinate, levallorphan, samidorphan, buprenorphine, dezocine, eptazocine, butorphanol, levorphanol, nalbuphine, pentazocine, phenazocine, norbinaltorphimine, or diprenorphine.

79. The method of claim 74, wherein the alkaloid product is a nor opioid alkaloid.

80. The method of claim 79, wherein the nor-opioid alkaloid is a norcodeine, noroxycodone, northebaine, norhydrocodone, nordihydro-codeine, nor-14-hydroxy-codeine, norcodeinone, nor-14-hydroxy-codeinone, normorphine, noroxymorphone, nororipavine, norhydro-morphone, nordihydro-morphine, nor-14-hydroxy-morphine, normorphinone, or nor-14-hydroxy-morphinone.

81. The method of claim 74, wherein the epimerase is an engineered epimerase.

82. The method of claim 81, wherein the engineered epimerase is a split epimerase.

83. The method of claim 81, wherein the engineered epimerase converts an (S)-1-benzylisoquinoline precursor to an (R)-1-benzylisoquinoline product.

84. The method of claim 83, wherein the engineered epimerase converts (S)-reticuline to (R)-reticuline.

85. The method of claim 83, wherein at least 50% of the (S)-1-benzylisoquinoline alkaloid molecules within the engineered non-plant cell are converted to the (R)-1-benzylisoquinoline product.

86. The method of claim 74, wherein the thebaine synthase is an engineered thebaine synthase.

87. The method of claim 74, wherein the precursor of a promorphinan molecule is selected from the group consisting ofreticuline, 3′ hydroxy-N-methylcoclaurine, coclaurine, norcoclaurine, norlaudanosoline, methylnorlaudanosoline, laudanosoline, methylnorlaudanosoline, norreticuline, 3′ hydroxy-N-methylcoclaurine, 4‘-’-Omethylaudanosoline, L-Dopa, tyrosine, dopamine, 3,4-dihydroxyphenylacetaldehyde (3,4-DHPA), hydroxyphenylpyruvate, prephenate, chorismate, 5-enolpyruvylshikimate-3-phosphate (EPSP), 3-deoxy-D-arabinoheptulosonate-7-phosphate (DAHP), erythrose-4-phosphate (E4P), phosphoenolpyruvate (PEP), and glucose.

88. The method of claim 74, wherein at least 50% of tetracyclic promorphinan precursor molecules within the engineered non-plant cell are converted to thebaine.

89. The method of claim 74, wherein the precursor of a promorphinan molecule is an (S)-substrate of Formula I: 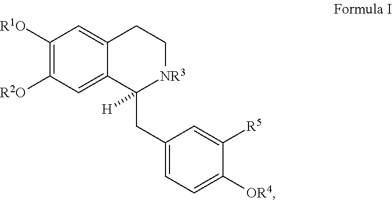
or a salt thereof, wherein:
R¹, R², R³, and R⁴ are independently selected from hydrogen and methyl; and
R⁵ is selected from hydrogen, hydroxy, and methoxy.

90. The method of claim 89, wherein at least one of R¹, R², R³, R⁴, and R⁵ is hydrogen.

91. The method of claim 74, wherein the precursor of a promorphinan molecule is (S)-substrate is a compound of Formula II: 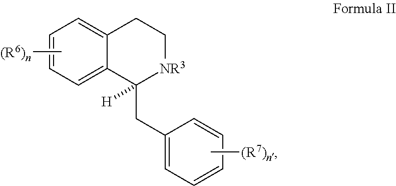
or a salt thereof, wherein:
R³ is selected from hydrogen and C₁-C₄ alkyl;
R⁶ and R⁷ are independently selected at each occurrence from hydroxy, fluoro, chloro, bromo, carboxaldehyde, C₁-C₄ acyl, C₁-C₄ alkyl, and C₁-C₄ alkoxy;
n is 0, 1, 2, 3, or 4; and
n′ is 0, 1, 2, 3, 4 or 5.

92. The method of claim 74, further comprising at least one modification selected from the group consisting of i) a BIA-generating modification, ii) an O-demethylation modification, iii) an N-demethylation modification, and iv) an N-linked modification.

93. The method of claim 74, wherein the engineered non-plant cell is a bacterial cell or a fungal cell.


SEQUENCE LISTING (153 sequences)

SEQ ID NO: 1 - protein, 901 aa, Papaver somniferum
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNHGNYTTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 2 - protein, 919 aa, Papaver somniferum
  VARIANT 209: note = Any amino acid
  VARIANT 226: note = Any amino acid
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNHGNYTTXLLLPQLAWRQPWKLYYXTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 3 - protein, 732 aa, Papaver somniferum
  VARIANT 725: note = Any amino acid
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNHGNYTTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRXVSKPWLH

SEQ ID NO: 4 - protein, 764 aa, Papaver somniferum
  VARIANT 6: note = Any amino acid
MRWHRXIDSYGLSSVPYGKYWRELRKVCVHNLLSNQQLLKFRHLIISQVDTSFNKLYELCKNSEDNQGNYPTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 5 - protein, 900 aa, Papaver somniferum
  source 1..900: note = subspecies: setigerum
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 6 - protein, 724 aa, Papaver somniferum
  source 1..724: note = subspecies: setigerum
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYTTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQALYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEE

SEQ ID NO: 7 - protein, 889 aa, Papaver bracteatum
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSSKKLQELMATANIPPAVNQVEMSPAFQQKKLREYCNANNILVSAVSILGSNGTPWGSNAVLGSEVLKQIAMAKGKSVAQVSMRWVYEQGASLVVKSFSEERLRENLNIFDWELTKEDNEKIGEIPQCRILTAYFLVSPNGPFKSQEELWDDKA

SEQ ID NO: 8 - protein, 889 aa, Papaver bracteatum
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSCKKLQELMATANIPPAVNQVEMSPAFQQKKLREYCNANNILVSAVSILGSNGTPWGSNAVLGSEVLKQIAMAKGKSVAQVSMRWVYEQGASLVVKSFSEERLRENLNIFDWELTKEDNEKIGEIPQCRILTAYFLVSPNGPFKSQEELWDDKA

SEQ ID NO: 9 - protein, 848 aa, Papaver bracteatum
SSPASSTETAVLCHQRQQSCALPISGLLHIFMNKNGLIHVTLGNMADKYGPIFSFPTGSHRILVVSSWEMVKECFTGNNDTAFSNRPIPLAFKTIFYACRGIDSYGLSSVPYGKYWRELRKVCVHNLLSNQQLLKFRHLIISQVDTSFNKLYELCKNSEDNQGMVRMDDWLAQLSFSVIGRIVCGFQSDPKTGAPSRVEQFKEAINEASYFMSTSPVSDNVPMLGWIDQLTGLTRNMTHCGKKLDLVVESIINDHRQKRRFSRTKGGDEKDDEQDDFIDICLSIMEQPQLPGNNNPPKIPIKSIVLDMIGAGTDTTKLTIIWTLSLLLNNPNVLAKAKQEVDAHFETKKRSTNEASVVVDFDDIGNLVYIQAIIKESMRLYPVSPVVERLSSEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 10 - protein, 847 aa, Papaver bracteatum
SSPASSTETAVLCHQRQQSCALPISGLLHIFMNKNGLIHVTLGNMADKYGPIFSFPTGSHRILVVSSWEMVKECFTGNNDTFFSNRPIPLAFKIIFYAGGVDSYGLALVPYGKYWRELRKICVHNLLSNQQLLKFRHLIISQVDTSFNKLYELCKNSEDNQGMVRMDDWLAQLSFSVIGRIVCGFQSDPKTGAPSRVEQFKEAINEASYFMSTSPVSDNVPMLGWIDQLTGLTRNMTHCGKKLDLVVESIINDHRQKRRFSRTKGGDEKDDEQDDFIDICLSIMEQPQLPGNNNPPKIPIKSIVLDMIGGGTDTTKLTTIWTLSLLLNNPHVLDKAKQEVDAHFLTKRRSTNDAAVVDFDDIRNLVYIQAIIKESMRLYPASPVVERLSGEDCVVGGFHVPAGTRLWVNVWKMQRDPNVWADPMVFRPERFLSHGQKKMVDVRGKNYELLPFGAGRRICPGISFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 11 - protein, 845 aa, Papaver bracteatum
SSPASSTETAVLCHQRQQSCALPISGLLHIFMNKNGLIHVTLGNMADKYGPIFSFPTGSHRILVVSSWEMVKECFTGNNDTFFSNRPIPLAFKIIFYAGGVDSYGLALVPYGKYWRELRKICVHNLLSNQQLLNFRHLIISQVDTSFNKLYDLSNKKKNTTTDSGTVRMDDWLAQLSFNVIGRIVCGFQTHTETSATSSVERFTEAIDEASRFMSIATVSDTFPWLGWIDQLTGLTRKMKHYGKKLDLVVESIIEDHRQNRRISGTKQGDDFIDICLSIMEQPQIIPGNNDPPRQIPIKSIVLDMIGGGTDTTKLTTTWTLSLLLNNPHVLEKAREEVDAHFGTKRRPTNDDAVMVEFDDIRNLVYIQAIIKESMRLYPASPVVERLSGEDCVVGGFHVPAGTRLWVNVWKMQRDPNVWADPMVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 12 - protein, 869 aa, Papaver bracteatum
VALRKKILKNYYSSSSSTATAVSHQWPKASRALPLIDLLHVFFNKTDLMHVTLGNMADKFGPIFSFPTGSHRTLVVSSWEKAKECFTGNNDIVFSGRPLPLAFKLIFYAGGIDSYGISQVPYGKKWRELRNICVHNILSNQQLLKFRHLMISQVDNSFNKLYEVCNSNKDEGDSATSTTAAGIVRMDDWLGKLAFDVIARIVCGFQSQTETSTTSSMERFTEAMDEASRFMSVTAVSDTVPWLGWIDQLTGLKRNMKHCGKKLNLVVKSIIEDHRQKRRLSSTKKGDENIIDEDEQDDFIDICLSIMEQPQLPGNNNPPKIPIKSIVLDMIGGGTDTTKLTTIWTLSLLLNNPHVLDKAKQEVDAHFLTKRRSTNDAAVVDFDDIRNLVYIQAIIKESMRLYPASPVVERLSGEDCVVGGFHVPAGTRLWVNVWKMQRDPNVWADPMVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLRSGKVMPVLGMGTFEKAGKGSERERLAILKAIEVGYRYFDTAAAYETEEVLGEAIAEALQLGLIKSRDELFISSMLWCTDAHPDRVLLALQNSLRNLKLEYVDLYMLPFPASLKPGKITMDIPEEDICPMDYRSVWSAMEECQNLGLTKSIGVSNFSCKKLEELMATANIPPAVNQVEMSPAFQQKKLREYCNANNILVSAVSILGSNGTPWGSNAVLGSEVLKKIAMAKGKSVAQVSMRWVYEQGASLVVKSFSEERLRENLNIFDWQLTKEDNEKIGEIPQCRILSAYFLVSPKGPFKSQEELWDDKA

SEQ ID NO: 13 - protein, 858 aa, Papaver bracteatum
  VARIANT 822: note = Any amino acid
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSSKKLQELMATANIPPAVNQVEMSPAFQQKKLREYCNANNILVSAVSILGSNGTPWGSNAVLGSEVLKQIAMAKGKSVAQVSMRWVXKFSAYAIVWSLFFGHRICITLYSFLIRNVAYICITY

SEQ ID NO: 14 - protein, 716 aa, Papaver bracteatum
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERDMESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRQVFLMQIRLIYICTYQQVHLNIYFQINEFVLCDMYRNLKLEY

SEQ ID NO: 15 - protein, 864 aa, Chelidonium majus
LNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYPTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAFLKAIEVGYRYFDTAAAYETEEFLGEAIAEALQLGLIKSRDELFITSKLWPCDAHPDLVVPALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA

SEQ ID NO: 16 - protein, 889 aa, synthetic construct; identical to SEQ ID NO: 8
  REGION 1..889: note = Description of Artificial Sequence: Synthetic polypeptide

SEQ ID NO: 17 - protein, 568 aa, synthetic construct
  REGION 1..568: note = Description of Artificial Sequence: Synthetic polypeptide
MELQYFSYFQPTSSVVALLLALVSILFSVVVLRKTFSNNYSSPASSTETAVLCHQRQQSCALPISGLLHVFMNKNGLIHVTLGNMADKYGPIFSFPTGSHRTLVVSSWEMVKECFTGNNDTAFSNRPIPLAFQTIFYACGGIDSYGLSSVPYGKYWRELRKVCVHNLLSNQQLLKFRHLIISQVDTSFNKLYELCKNSEDNQGMVRMDDWLAQLSFNVIGRIVCGFQSDPKTGAPSRVEQFKEVINEASYFMSTSPVSDNVPMLGWIDQLTGLTRNMKHCGKKLDLVVESIIKDHRQKRRFSRTKGGDEKDDEQDDFIDICLSIMEQPQLPGNNSPPQIPIKSIVLDMIGGGTDTTKLTTIWTLSLLLNNPHVLDKAKQEVDAHFRKKRRSTDDAAAAVVDFDDIRNLVYIQAIIKESMRLYPASPVVERLSGEDCVVGGFHVPAGTRLWANVWKMQRDPKVWDDPLVFRPERFLSDEQKMVDVRGQNYELLPFGAGRRICPGVSFSLDLMQLVLTRLILEFEMKSPSGKVDMTATPGLMSYKVVPLDILLTHRRIKSCVQLASSERD

SEQ ID NO: 18 - protein, 321 aa, synthetic construct
  REGION 1..321: note = Description of Artificial Sequence: Synthetic polypeptide
MESSGVPVITLSSGKVMPVLGMGTFEKVGKGSERERLAILKAIEVGYRYFDTAAAYETEEVLGEAIAEALQLGLIESRDELFISSMLWCTDAHPDRVLLALQNSLRNLKLEYLDLYMLPFPASLKPGKITMDIPEEDICRMDYRSVWSAMEECQNLGFTKSIGVSNFSCKKLQELMATANIPPAVNQVEMSPAFQQKKLREYCNANNILVSAVSILGSNGTPWGSNAVLGSEVLKQIAMAKGKSVAQVSMRWVYEQGASLVVKSFSEERLRENLNIFDWELTKEDNEKIGEIPQCRILTAYFLVSPNGPFKSQEELWDDKA

SEQ ID NO: 19 - DNA, 682 bp, Saccharomyces cerevisiae
  source 1..682: mol_type = genomic DNA
ttcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 20 - DNA, 289 bp, Saccharomyces cerevisiae
  source 1..289: mol_type = genomic DNA
gagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaata

SEQ ID NO: 21 - DNA, 413 bp, Saccharomyces cerevisiae
  source 1..413: mol_type = genomic DNA
catagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 22 - DNA, 274 bp, Saccharomyces cerevisiae
  source 1..274: mol_type = genomic DNA
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttg

SEQ ID NO: 23 - DNA, 150 bp, Saccharomyces cerevisiae
  source 1..150: mol_type = genomic DNA
gcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggta

SEQ ID NO: 24 - DNA, 8014 bp, synthetic construct
  misc_feature 1..8014: note = Description of Artificial Sequence: Synthetic polynucleotide
  source 1..8014: mol_type = other DNA
cctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgattcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggaacttcagtacttctcctattttcaacccacttcatctgtcgtagccctactactagcactagtgagtattttatttagcgtagttgttttgaggaagactttcagtaacaattactccagccccgcgtcaagtacggaaaccgctgtgctgtgtcatcagaggcaacagagttgcgccctacctatcagcggccttcttcacgtgttcatgaataagaacggcctgattcatgtcaccttgggaaatatggctgacaaatatggccctatcttcagttttccgacaggcagccaccgtactttagtagtcagttcctgggaaatggtgaaagagtgtttcaccggtaataacgacacggcattctccaacagaccaatccctttggcttttcaaaccatattctacgcctgtggcggcattgattcttacggtttaagtagtgtcccgtatggtaaatactggagggagttgagaaaggtgtgtgttcacaacctgctgagtaatcagcaattgctgaagttcagacatcttataatctcccaagtggatacgtcttttaacaagttgtatgagctgtgtaagaactctgaagataatcaaggtatggtaaggatggatgattggctagctcaactttcctttaacgtcatcggtaggatcgtttgcggattccagtctgacccaaagacgggtgcaccttcaagggtagaacagtttaaggaagtcataaatgaggcgtcatattttatgtcaacaagtccagtctccgataacgtaccaatgttgggatggatcgaccaattgaccggtctgacgaggaacatgaagcattgtgggaagaagcttgacttagtagtggagtcaattatcaaggaccataggcaaaagagacgtttttcacgtacaaaaggtggcgatgagaaggatgacgaacaggacgactttattgatatttgcttgagcatcatggagcagccacagttgcccgggaacaattctccccctcaaattccgatcaaatctatcgtgctagacatgattgggggtggtaccgacactacgaaacttacaaccatatggaccctatcacttttgttgaacaatcctcacgtgttagataaagctaaacaagaggtcgacgctcactttcgtaaaaagagaagatcaacagatgacgcagcagcggcagtcgttgattttgacgacataagaaatttagtatacatccaagccatcattaaagaaagtatgaggctttatccagccagcccggtggttgagcgtctttccggcgaggattgcgttgttggaggttttcacgtgcctgctggtacgagactatgggctaacgtttggaagatgcaaagagatcccaaagtttgggacgatcctctagtattcagacctgaaaggtttttgagcgacgagcaaaagatggtagacgttcgtggccaaaactatgaacttctgccattcggcgcaggaagaagaatctgtccaggcgtttcctttagtcttgaccttatgcaacttgtcctaaccaggttaatcctagagttcgaaatgaagtccccgtccggcaaggtagatatgaccgcaactccaggactaatgtcttacaaggtggttccattggacatattgctgactcaccgtcgtatcaagtcatgcgttcaattggcgtcttctgaacgtgatatggaaagttctggggtgcctgtgatcacattgtcctcaggtaaagtaatgcccgtactgggcatgggaaccttcgaaaaggtgggtaaggggtctgaacgtgagcgtttagccattcttaaagcgatcgaagttggttaccgttactttgataccgcagcggcatatgaaacggaagaagttctaggggaagccattgctgaagctttacaattgggtctgatagagagccgtgacgagctgttcatcagctcaatgctttggtgcaccgacgcacatccagaccgtgtgctacttgctctgcaaaacagtctgagaaatctaaaacttgaatatctagacctatatatgttgccgtttcctgccagccttaagccgggcaaaattacgatggatattcctgaggaggatatttgccgtatggattatcgttcagtctggagcgccatggaagagtgtcaaaacttaggatttactaaaagtattggtgtaagcaacttttcttgcaagaaattacaagaattaatggccactgcaaatatcccgcccgcggtaaatcaagtagagatgtcaccagctttccaacagaaaaaactgagggaatattgtaacgcaaacaacatattggtatccgcagtaagcattctgggatcaaacgggacgccctggggtagtaatgctgttcttggaagcgaagttttgaaacagatcgcgatggcgaaaggcaaaagcgttgcgcaagtcagtatgaggtgggtctatgagcagggcgcgtctttagtagtcaagagtttctctgaagaacgtttaagagaaaacctgaatatttttgactgggagcttacgaaagaagacaatgagaagataggcgaaatcccgcaatgtagaatccttactgcgtacttccttgtctccccgaacggcccgtttaaatctcaggaagagctttgggatgacaaggcataaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgc

SEQ ID NO: 25 - DNA, 7621 bp, synthetic construct
  misc_feature 1..7621: note = Description of Artificial Sequence: Synthetic polynucleotide
  source 1..7621: mol_type = other DNA
cctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgagagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataatggaacttcagtacttctcctattttcaacccacttcatctgtcgtagccctactactagcactagtgagtattttatttagcgtagttgttttgaggaagactttcagtaacaattactccagccccgcgtcaagtacggaaaccgctgtgctgtgtcatcagaggcaacagagttgcgccctacctatcagcggccttcttcacgtgttcatgaataagaacggcctgattcatgtcaccttgggaaatatggctgacaaatatggccctatcttcagttttccgacaggcagccaccgtactttagtagtcagttcctgggaaatggtgaaagagtgtttcaccggtaataacgacacggcattctccaacagaccaatccctttggcttttcaaaccatattctacgcctgtggcggcattgattcttacggtttaagtagtgtcccgtatggtaaatactggagggagttgagaaaggtgtgtgttcacaacctgctgagtaatcagcaattgctgaagttcagacatcttataatctcccaagtggatacgtcttttaacaagttgtatgagctgtgtaagaactctgaagataatcaaggtatggtaaggatggatgattggctagctcaactttcctttaacgtcatcggtaggatcgtttgcggattccagtctgacccaaagacgggtgcaccttcaagggtagaacagtttaaggaagtcataaatgaggcgtcatattttatgtcaacaagtccagtctccgataacgtaccaatgttgggatggatcgaccaattgaccggtctgacgaggaacatgaagcattgtgggaagaagcttgacttagtagtggagtcaattatcaaggaccataggcaaaagagacgtttttcacgtacaaaaggtggcgatgagaaggatgacgaacaggacgactttattgatatttgcttgagcatcatggagcagccacagttgcccgggaacaattctccccctcaaattccgatcaaatctatcgtgctagacatgattgggggtggtaccgacactacgaaacttacaaccatatggaccctatcacttttgttgaacaatcctcacgtgttagataaagctaaacaagaggtcgacgctcactttcgtaaaaagagaagatcaacagatgacgcagcagcggcagtcgttgattttgacgacataagaaatttagtatacatccaagccatcattaaagaaagtatgaggctttatccagccagcccggtggttgagcgtctttccggcgaggattgcgttgttggaggttttcacgtgcctgctggtacgagactatgggctaacgtttggaagatgcaaagagatcccaaagtttgggacgatcctctagtattcagacctgaaaggtttttgagcgacgagcaaaagatggtagacgttcgtggccaaaactatgaacttctgccattcggcgcaggaagaagaatctgtccaggcgtttcctttagtcttgaccttatgcaacttgtcctaaccaggttaatcctagagttcgaaatgaagtccccgtccggcaaggtagatatgaccgcaactccaggactaatgtcttacaaggtggttccattggacatattgctgactcaccgtcgtatcaagtcatgcgttcaattggcgtcttctgaacgtgatatggaaagttctggggtgcctgtgatcacattgtcctcaggtaaagtaatgcccgtactgggcatgggaaccttcgaaaaggtgggtaaggggtctgaacgtgagcgtttagccattcttaaagcgatcgaagttggttaccgttactttgataccgcagcggcatatgaaacggaagaagttctaggggaagccattgctgaagctttacaattgggtctgatagagagccgtgacgagctgttcatcagctcaatgctttggtgcaccgacgcacatccagaccgtgtgctacttgctctgcaaaacagtctgagaaatctaaaacttgaatatctagacctatatatgttgccgtttcctgccagccttaagccgggcaaaattacgatggatattcctgaggaggatatttgccgtatggattatcgttcagtctggagcgccatggaagagtgtcaaaacttaggatttactaaaagtattggtgtaagcaacttttcttgcaagaaattacaagaattaatggccactgcaaatatcccgcccgcggtaaatcaagtagagatgtcaccagctttccaacagaaaaaactgagggaatattgtaacgcaaacaacatattggtatccgcagtaagcattctgggatcaaacgggacgccctggggtagtaatgctgttcttggaagcgaagttttgaaacagatcgcgatggcgaaaggcaaaagcgttgcgcaagtcagtatgaggtgggtctatgagcagggcgcgtctttagtagtcaagagtttctctgaagaacgtttaagagaaaacctgaatatttttgactgggagcttacgaaagaagacaatgagaagataggcgaaatcccgcaatgtagaatccttactgcgtacttccttgtctccccgaacggcccgtttaaatctcaggaagagctttgggatgacaaggcataaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgc

SEQ ID NO: 26 - DNA, 8187 bp, synthetic construct
  misc_feature 1..8187: note = Description of Artificial Sequence: Synthetic polynucleotide
  source 1..8187: mol_type = other DNA
cctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgagagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataatggaacttcagtacttctcctattttcaacccacttcatctgtcgtagccctactactagcactagtgagtattttatttagcgtagttgttttgaggaagactttcagtaacaattactccagccccgcgtcaagtacggaaaccgctgtgctgtgtcatcagaggcaacagagttgcgccctacctatcagcggccttcttcacgtgttcatgaataagaacggcctgattcatgtcaccttgggaaatatggctgacaaatatggccctatcttcagttttccgacaggcagccaccgtactttagtagtcagttcctgggaaatggtgaaagagtgtttcaccggtaataacgacacggcattctccaacagaccaatccctttggcttttcaaaccatattctacgcctgtggcggcattgattcttacggtttaagtagtgtcccgtatggtaaatactggagggagttgagaaaggtgtgtgttcacaacctgctgagtaatcagcaattgctgaagttcagacatcttataatctcccaagtggatacgtcttttaacaagttgtatgagctgtgtaagaactctgaagataatcaaggtatggtaaggatggatgattggctagctcaactttcctttaacgtcatcggtaggatcgtttgcggattccagtctgacccaaagacgggtgcaccttcaagggtagaacagtttaaggaagtcataaatgaggcgtcatattttatgtcaacaagtccagtctccgataacgtaccaatgttgggatggatcgaccaattgaccggtctgacgaggaacatgaagcattgtgggaagaagcttgacttagtagtggagtcaattatcaaggaccataggcaaaagagacgtttttcacgtacaaaaggtggcgatgagaaggatgacgaacaggacgactttattgatatttgcttgagcatcatggagcagccacagttgcccgggaacaattctccccctcaaattccgatcaaatctatcgtgctagacatgattgggggtggtaccgacactacgaaacttacaaccatatggaccctatcacttttgttgaacaatcctcacgtgttagataaagctaaacaagaggtcgacgctcactttcgtaaaaagagaagatcaacagatgacgcagcagcggcagtcgttgattttgacgacataagaaatttagtatacatccaagccatcattaaagaaagtatgaggctttatccagccagcccggtggttgagcgtctttccggcgaggattgcgttgttggaggttttcacgtgcctgctggtacgagactatgggctaacgtttggaagatgcaaagagatcccaaagtttgggacgatcctctagtattcagacctgaaaggtttttgagcgacgagcaaaagatggtagacgttcgtggccaaaactatgaacttctgccattcggcgcaggaagaagaatctgtccaggcgtttcctttagtcttgaccttatgcaacttgtcctaaccaggttaatcctagagttcgaaatgaagtccccgtccggcaaggtagatatgaccgcaactccaggactaatgtcttacaaggtggttccattggacatattgctgactcaccgtcgtatcaagtcatgcgttcaattggcgtcttctgaacgtgattaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtacatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatggaaagttctggggtgcctgtgatcacattgtcctcaggtaaagtaatgcccgtactgggcatgggaaccttcgaaaaggtgggtaaggggtctgaacgtgagcgtttagccattcttaaagcgatcgaagttggttaccgttactttgataccgcagcggcatatgaaacggaagaagttctaggggaagccattgctgaagctttacaattgggtctgatagagagccgtgacgagctgttcatcagctcaatgctttggtgcaccgacgcacatccagaccgtgtgctacttgctctgcaaaacagtctgagaaatctaaaacttgaatatctagacctatatatgttgccgtttcctgccagccttaagccgggcaaaattacgatggatattcctgaggaggatatttgccgtatggattatcgttcagtctggagcgccatggaagagtgtcaaaacttaggatttactaaaagtattggtgtaagcaacttttcttgcaagaaattacaagaattaatggccactgcaaatatcccgcccgcggtaaatcaagtagagatgtcaccagctttccaacagaaaaaactgagggaatattgtaacgcaaacaacatattggtatccgcagtaagcattctgggatcaaacgggacgccctggggtagtaatgctgttcttggaagcgaagttttgaaacagatcgcgatggcgaaaggcaaaagcgttgcgcaagtcagtatgaggtgggtctatgagcagggcgcgtctttagtagtcaagagtttctctgaagaacgtttaagagaaaacctgaatatttttgactgggagcttacgaaagaagacaatgagaagataggcgaaatcccgcaatgtagaatccttactgcgtacttccttgtctccccgaacggcccgtttaaatctcaggaagagctttgggatgacaaggcataaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgc

SEQ ID NO: 27 - DNA, 8580 bp, synthetic construct
  misc_feature 1..8580: note = Description of Artificial Sequence: Synthetic polynucleotide
  source 1..8580: mol_type = other DNA
cctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgattcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggaacttcagtacttctcctattttcaacccacttcatctgtcgtagccctactactagcactagtgagtattttatttagcgtagttgttttgaggaagactttcagtaacaattactccagccccgcgtcaagtacggaaaccgctgtgctgtgtcatcagaggcaacagagttgcgccctacctatcagcggccttcttcacgtgttcatgaataagaacggcctgattcatgtcaccttgggaaatatggctgacaaatatggccctatcttcagttttccgacaggcagccaccgtactttagtagtcagttcctgggaaatggtgaaagagtgtttcaccggtaataacgacacggcattctccaacagaccaatccctttggcttttcaaaccatattctacgcctgtggcggcattgattcttacggtttaagtagtgtcccgtatggtaaatactggagggagttgagaaaggtgtgtgttcacaacctgctgagtaatcagcaattgctgaagttcagacatcttataatctcccaagtggatacgtcttttaacaagttgtatgagctgtgtaagaactctgaagataatcaaggtatggtaaggatggatgattggctagctcaactttcctttaacgtcatcggtaggatcgtttgcggattccagtctgacccaaagacgggtgcaccttcaagggtagaacagtttaaggaagtcataaatgaggcgtcatattttatgtcaacaagtccagtctccgataacgtaccaatgttgggatggatcgaccaattgaccggtctgacgaggaacatgaagcattgtgggaagaagcttgacttagtagtggagtcaattatcaaggaccataggcaaaagagacgtttttcacgtacaaaaggtggcgatgagaaggatgacgaacaggacgactttattgatatttgcttgagcatcatggagcagccacagttgcccgggaacaattctccccctcaaattccgatcaaatctatcgtgctagacatgattgggggtggtaccgacactacgaaacttacaaccatatggaccctatcacttttgttgaacaatcctcacgtgttagataaagctaaacaagaggtcgacgctcactttcgtaaaaagagaagatcaacagatgacgcagcagcggcagtcgttgattttgacgacataagaaatttagtatacatccaagccatcattaaagaaagtatgaggctttatccagccagcccggtggttgagcgtctttccggcgaggattgcgttgttggaggttttcacgtgcctgctggtacgagactatgggctaacgtttggaagatgcaaagagatcccaaagtttgggacgatcctctagtattcagacctgaaaggtttttgagcgacgagcaaaagatggtagacgttcgtggccaaaactatgaacttctgccattcggcgcaggaagaagaatctgtccaggcgtttcctttagtcttgaccttatgcaacttgtcctaaccaggttaatcctagagttcgaaatgaagtccccgtccggcaaggtagatatgaccgcaactccaggactaatgtcttacaaggtggttccattggacatattgctgactcaccgtcgtatcaagtcatgcgttcaattggcgtcttctgaacgtgattaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtacatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatggaaagttctggggtgcctgtgatcacattgtcctcaggtaaagtaatgcccgtactgggcatgggaaccttcgaaaaggtgggtaaggggtctgaacgtgagcgtttagccattcttaaagcgatcgaagttggttaccgttactttgataccgcagcggcatatgaaacggaagaagttctaggggaagccattgctgaagctttacaattgggtctgatagagagccgtgacgagctgttcatcagctcaatgctttggtgcaccgacgcacatccagaccgtgtgctacttgctctgcaaaacagtctgagaaatctaaaacttgaatatctagacctatatatgttgccgtttcctgccagccttaagccgggcaaaattacgatggatattcctgaggaggatatttgccgtatggattatcgttcagtctggagcgccatggaagagtgtcaaaacttaggatttactaaaagtattggtgtaagcaacttttcttgcaagaaattacaagaattaatggccactgcaaatatcccgcccgcggtaaatcaagtagagatgtcaccagctttccaacagaaaaaactgagggaatattgtaacgcaaacaacatattggtatccgcagtaagcattctgggatcaaacgggacgccctggggtagtaatgctgttcttggaagcgaagttttgaaacagatcgcgatggcgaaaggcaaaagcgttgcgcaagtcagtatgaggtgggtctatgagcagggcgcgtctttagtagtcaagagtttctctgaagaacgtttaagagaaaacctgaatatttttgactgggagcttacgaaagaagacaatgagaagataggcgaaatcccgcaatgtagaatccttactgcgtacttccttgtctccccgaacggcccgtttaaatctcaggaagagctttgggatgacaaggcataaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgc

SEQ ID NO: 28 - DNA, 8456 bp, synthetic construct
  misc_feature 1..8456: note = Description of Artificial Sequence: Synthetic polynucleotide
  source 1..8456: mol_type = other DNA
cctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgagagcgttggttggtggatcaagcccacgcgtaggcaatcctcgagcagatccgccaggcgtgtatatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataatggaacttcagtacttctcctattttcaacccacttcatctgtcgtagccctactactagcactagtgagtattttatttagcgtagttgttttgaggaagactttcagtaacaattactccagccccgcgtcaagtacggaaaccgctgtgctgtgtcatcagaggcaacagagttgcgccctacctatcagcggccttcttcacgtgttcatgaataagaacggcctgattcatgtcaccttgggaaatatggctgacaaatatggccctatcttcagttttccgacaggcagccaccgtactttagtagtcagttcctgggaaatggtgaaagagtgtttcaccggtaataacgacacggcattctccaacagaccaatccctttggcttttcaaaccatattctacgcctgtggcggcattgattcttacggtttaagtagtgtcccgtatggtaaatactggagggagttgagaaaggtgtgtgttcacaacctgctgagtaatcagcaattgctgaagttcagacatcttataatctcccaagtggatacgtcttttaacaagttgtatgagctgtgtaagaactctgaagataatcaaggtatggtaaggatggatgattggctagctcaactttcctttaacgtcatcggtaggatcgtttgcggattccagtctgacccaaagacgggtgcaccttcaagggtagaacagtttaaggaagtcataaatgaggcgtcatattttatgtcaacaagtccagtctccgataacgtaccaatgttgggatggatcgaccaattgaccggtctgacgaggaacatgaagcattgtgggaagaagcttgacttagtagtggagtcaattatcaaggaccataggcaaaagagacgtttttcacgtacaaaaggtggcgatgagaaggatgacgaacaggacgactttattgatatttgcttgagcatcatggagcagccacagttgcccgggaacaattctccccctcaaattccgatcaaatctatcgtgctagacatgattgggggtggtaccgacactacgaaacttacaaccatatggaccctatcacttttgttgaacaatcctcacgtgttagataaagctaaacaagaggtcgacgctcactttcgtaaaaagagaagatcaacagatgacgcagcagcggcagtcgttgattttgacgacataagaaatttagtatacatccaagccatcattaaagaaagtatgaggctttatccagccagcccggtggttgagcgtctttccggcgaggattgcgttgttggaggttttcacgtgcctgctggtacgagactatgggctaacgtttggaagatgcaaagagatcccaaagtttgggacgatcctctagtattcagacctgaaaggtttttgagcgacgagcaaaagatggtagacgttcgtggccaaaactatgaacttctgccattcggcgcaggaagaagaatctgtccaggcgtttcctttagtcttgaccttatgcaacttgtcctaaccaggttaatcctagagttcgaaatgaagtccccgtccggcaaggtagatatgaccgcaactccaggactaatgtcttacaaggtggttccattggacatattgctgactcaccgtcgtatcaagtcatgcgttcaattggcgtcttctgaacgtgattaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtattcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggaaagttctggggtgcctgtgatcacattgtcctcaggtaaagtaatgcccgtactgggcatgggaaccttcgaaaaggtgggtaaggggtctgaacgtgagcgtttagccattcttaaagcgatcgaagttggttaccgttactttgataccgcagcggcatatgaaacggaagaagttctaggggaagccattgctgaagctttacaattgggtctgatagagagccgtgacgagctgttcatcagctcaatgctttggtgcaccgacgcacatccagaccgtgtgctacttgctctgcaaaacagtctgagaaatctaaaacttgaatatctagacctatatatgttgccgtttcctgccagccttaagccgggcaaaattacgatggatattcctgaggaggatatttgccgtatggattatcgttcagtctggagcgccatggaagagtgtcaaaacttaggatttactaaaagtattggtgtaagcaacttttcttgcaagaaattacaagaattaatggccactgcaaatatcccgcccgcggtaaatcaagtagagatgtcaccagctttccaacagaaaaaactgagggaatattgtaacgcaaacaacatattggtatccgcagtaagcattctgggatcaaacgggacgccctggggtagtaatgctgttcttggaagcgaagttttgaaacagatcgcgatggcgaaaggcaaaagcgttgcgcaagtcagtatgaggtgggtctatgagcagggcgcgtctttagtagtcaagagtttctctgaagaacgtttaagagaaaacctgaatatttttgactgggagcttacgaaagaagacaatgagaagataggcgaaatcccgcaatgtagaatccttactgcgtacttccttgtctccccgaacggcccgtttaaatctcaggaagagctttgggatgacaaggcataaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgc

SEQ ID NO: 29 - DNA, 8456 bp, synthetic construct
  misc_feature 1..8456: note = Description of Artificial Sequence: Synthetic polynucleotide
  source 1..8456: mol_type = other DNA
cctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgattcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatggaacttcagtacttctcctattttcaacccacttcatctgtcgtagccctactactagcactagtgagtattttatttagcgtagttgttttgaggaagactttcagtaacaattactccagccccgcgtcaagtacggaaaccgctgtgctgtgtcatcagaggcaacagagttgcgccctacctatcagcggccttcttcacgtgttcatgaataagaacggcctgattcatgtcaccttgggaaatatggctgacaaatatggccctatcttcagttttccgacaggcagccaccgtactttagtagtcagttcctgggaaatggtgaaagagtgtttcaccggtaataacgacacggcattctccaacagaccaatccctttggcttttcaaaccatattctacgcctgtggcggcattgattcttacggtttaagtagtgtcccgtatggtaaatactggagggagttgagaaaggtgtgtgttcacaacctgctgagtaatcagcaattgctgaagttcagacatcttataatctcccaagtggatacgtcttttaacaagttgtatgagctgtgtaagaactctgaagataatcaaggtatggtaaggatggatgattggctagctcaactttcctttaacgtcatcggtaggatcgtttgcggattccagtctgacccaaagacgggtgcaccttcaagggtagaacagtttaaggaagtcataaatgaggcgtcatattttatgtcaacaagtccagtctccgataacgtaccaatgttgggatggatcgaccaattgaccggtctgacgaggaacatgaagcattgtgggaagaagcttgacttagtagtggagtcaattatcaaggaccataggcaaaagagacgtttttcacgtacaaaaggtggcgatgagaaggatgacgaacaggacgactttattgatatttgcttgagcatcatggagcagccacagttgcccgggaacaattctccccctcaaattccgatcaaatctatcgtgctagacatgattgggggtggtaccgacactacgaaacttacaaccatatggaccctatcacttttgttgaacaatcctcacgtgttagataaagctaaacaagaggtcgacgctcactttcgtaaaaagagaagatcaacagatgacgcagcagcggcagtcgttgattttgacgacataagaaatttagtatacatccaagccatcattaaagaaagtatgaggctttatccagccagcccggtggttgagcgtctttccggcgaggattgcgttgttggaggttttcacgtgcctgctggtacgagactatgggctaacgtttggaagatgcaaagagatcccaaagtttgggacgatcctctagtattcagacctgaaaggtttttgagcgacgagcaaaagatggtagacgttcgtggccaaaactatgaacttctgccattcggcgcaggaagaagaatctgtccaggcgtttcctttagtcttgaccttatgcaacttgtcctaaccaggttaatcctagagttcgaaatgaagtccccgtccggcaaggtagatatgaccgcaactccaggactaatgtcttacaaggtggttccattggacatattgctgactcaccgtcgtatcaagtcatgcgttcaattggcgtcttctgaacgtgattaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtagagcgttggttggtggatcaagcccacgcgtaggcaatcctcgagcagatccgccaggcgtgtatatatagcgtggatggccaggcaactttagtgctgacacatacaggcatatatatatgtgtgcgacaacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataatggaaagttctggggtgcctgtgatcacattgtcctcaggtaaagtaatgcccgtactgggcatgggaaccttcgaaaaggtgggtaaggggtctgaacgtgagcgtttagccattcttaaagcgatcgaagttggttaccgttactttgataccgcagcggcatatgaaacggaagaagttctaggggaagccattgctgaagctttacaattgggtctgatagagagccgtgacgagctgttcatcagctcaatgctttggtgcaccgacgcacatccagaccgtgtgctacttgctctgcaaaacagtctgagaaatctaaaacttgaatatctagacctatatatgttgccgtttcctgccagccttaagccgggcaaaattacgatggatattcctgaggaggatatttgccgtatggattatcgttcagtctggagcgccatggaagagtgtcaaaacttaggatttactaaaagtattggtgtaagcaacttttcttgcaagaaattacaagaattaatggccactgcaaatatcccgcccgcggtaaatcaagtagagatgtcaccagctttccaacagaaaaaactgagggaatattgtaacgcaaacaacatattggtatccgcagtaagcattctgggatcaaacgggacgccctggggtagtaatgctgttcttggaagcgaagttttgaaacagatcgcgatggcgaaaggcaaaagcgttgcgcaagtcagtatgaggtgggtctatgagcagggcgcgtctttagtagtcaagagtttctctgaagaacgtttaagagaaaacctgaatatttttgactgggagcttacgaaagaagacaatgagaagataggcgaaatcccgcaatgtagaatccttactgcgtacttccttgtctccccgaacggcccgtttaaatctcaggaagagctttgggatgacaaggcataaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgc

SEQ ID NO: 30 - protein, 153 aa, Papaver bracteatum
MAPRGVSGLVGKLSTELDVNCDAEKYYNMYKNGEDVQKAVPHLCMDVKVISGDATRSGCIKEWNVNIDGKTIRSVEETTHNDETKTLRHRVFEGDMMKDYKKFDTIMEVNPKPDGNGCVVTRSIEYEKVNENSPTPFDYLQFGHQAMEDMNKY

SEQ ID NO: 31 - protein, 144 aa, Papaver somniferum
  source 1..144: note = subspecies: setigerum
MLVGKLSTELEVDCDAEKYYNMYKHGEDVKKALCVDVKVISGDPTRSGCIKEWNVNIDGKTIRSVEETTHNDETKTLRHRVFEGDMMKDFKKFDTIMVVNPKPDGNGCVVTRSIEYEKTNENSPTPFDYLQFGHQAIEDMNKYL

SEQ ID NO: 32 - protein, 141 aa, Papaver somniferum
  source 1..141: note = subspecies: setigerum
MLVGKLSTELEVDCDAEKYYNMYKHGEDKRQCVDVKVISGDPTRSGCIKEWNVNIDGKTIRSVEETTHNDETKTLRHRVFEGDMMKDFKKFDTIMVVNPKPDGNGCVVTRSIEYEKTNENSPTPFDYLQFGHQAIEDMNKY

SEQ ID NO: 33 - protein, 147 aa, Papaver somniferum
  source 1..147: note = subspecies: setigerum
MLVGKLSTELEVDCDAEKYYNMYKHGEDVKKAVPHLCVDVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYL

SEQ ID NO: 34 - protein, 109 aa, Papaver somniferum
  source 1..109: note = subspecies: setigerum
MVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYL

SEQ ID NO: 35 - protein, 180 aa, Papaver somniferum
MDSINSSIYFCAYFRELIIKLLMAPPGVSGLVGKLSTELEVNCDAEKYYNMYKHGEDVQKAVPHLCVDVKVISGDPTRSGCIKEWNVNIDGKTIRSVEETTHNDETKTLRHRVFEGDVMKDFKKFDTIMVVNPKPDGNGCVVTRSIEYEKTNDNSPTPFDYLQFGHQAIEDMNKYLRDSE

SEQ ID NO: 36 - protein, 164 aa, Papaver somniferum
MNFFIKDHLYICLVGKLSTELEVDCDAEKYYNMYKHGEDVKKAVPHLCVDVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYLRDSESN

SEQ ID NO: 37 - protein, 160 aa, Papaver somniferum
MAPLGVSGLVGKLSTELEVDCDAEKYYNMYKHGEDVKKAVPHLCVDVKIISGDPTSSGCIKEWNVNIDGKTIRSVEETTHDDETKTLRHRVFEGDVMKDFKKFDTIMVVNPKPDGNGCVVTRSIEYEKTNENSPTPFDYLQFGHQAIEDMNKYLRDSESN

SEQ ID NO: 38 - protein, 400 aa, Papaver somniferum
MMKVCVSSREKIKPSRPTPGHLKTHKLSFLDQVAARIYVPLLLYYAGNKENVDTDTRCNIIKKSLAETLTKFYILAGKIVNDEIERFVNCNDDGVDFCVTKVSNCQLFQVIKRPDIFDQVTLFLPFDPCDNEITASGDFLLSVQVNVFEDCRGMVIGLCINHKVADASSITTFVNYWATIARGLVLNVDDRQIQDPCFQVQSIFPQKEKGIGFKISSSSIDGTLVTKKFGFEASKLAELKERCKFAGATEDIRGGYKPNRVEALSTFLWKCFIDIDQAKTKAAAPARVYLASNAVNIRSRIVPQLPTSSFGNMVAITDAIFTVNSNENNGINDPYYPKLVQKFRDAVKRVDGEYIEALQSTDLLLNNVTKLFKHILNGQTLSISFTSWCRFPFYDTDLLD

SEQ ID NO: 39 - protein, 386 aa, Papaver somniferum
MKVQVISKELIKPSTPTPPRLRNFKLSLLDQLLPPFYVPIIIFYPANDDHESNNNDQCIKANILKKSLSETLTRFYPIAGRIRDKILVECNDEGVHYIEAKVNAVMSDFMSLDVIHQLHPSYITLDDLAEEAQLAVQVTMFDCGGIALSICSSHKIIDGCTSTTFLNSWAATARAPSNPEIVYPTFDAAAIFPAQPSGVQVSTLESDDRLQGENVVTKRFLFSASKITALRARIAESRSSNILSKYPSRSEAVSALVWKSFMETSRVKVTREHTFSAEASTKPIVRSIANFVVNLRTRLNPPLPNVSFGNIIMDATAESLIIDNGENTLGFVETLDGLISQLRLGVTKMDDEYVRKLREDDVEFLKSLDEASHPSNGEGDGNGERV

SEQ ID NO: 40 - protein, 525 aa, Papaver somniferum
  source 1..525: note = subspecies: setigerum
MNDTMKIEVVSKESIKPSYPTPNNLKIHNLSNLDQLIPAFYMDHILYYPSLDSNDSSLGDDEEDKKMIFSASSRHRCDVVKKSLAETLTRYYPLAGRIKDEKSVECNDEGVDYIEARVVGITVSQVIQLASSDIEVMEPFLPYEPYGGTGSAFRRAGIHSNSKPLLKIQVNVFDCGGMVICLSGSHKVIDATSILNFVNDWAATARGGFDTHDDELKVAVVDKPCYIFSSMFPPTSFGNQEEKDTADQAQLVPDRIEIVTKRFVFKDSSIAKLKKKCIHVNTNNGSDHQVDKQEHNMQQMPSRIEALTSLIWMCFMDVDRRFRVKQIDDAVSPVNTVNEVSLPKQVQYVAGFAINLRTRTIQPLPTNSFGNMTDTAIAEVTLNLTGSDHFNNEKGIRDQSQNYPELVSKIKDSIKLVDNKHIEAMKRNLAISCNNIKMHQMMKESTFDQNTRELLMFSSWCRFPIYEADFGWGKPSWASITKLLYKNCVMFLDTSSGDGIEAWVSLKEEDMVEFERHEELVALAS

SEQ ID NO: 41 - protein, 459 aa, Papaver somniferum
MKVQVISKEIIKPSSPTPPHLRNFKLSLLDQILPPFYVPIVMFYPAGDDYVTNNNIHDQSSKSEFLKKSLSETLTRFYPIAGRIKDNILIDCNNEGVDYIEAKVNGIMSDFMSVDVVHQLHPSHIMLDDVAKEAQLAVQVNLFDCGGIAISISMSHKIVDACTAITFINGWAATARAAPKQEIVCPTFDSAAIFPALPPGVQVSSLESDDSVQGVNVVTKMFAFTAPKIASLRARIAELRSSSDGLSKYPTRTEALSALVWKSFIRTSRVKAARKYSLSPASTKPVIKSVANYAVNLRTRLNPPLPQVSFGNILMDATAESTTTIDDDDSHEFADTLAGLIGQLRLGVSRINGDYIRKLQEGDLAFLKSLDEASHDSNGEKVQICWISSLCRFPFYEADFGWGKPSWVALNTNAEYKNSLFLMDTKCGTGIEAWVSLEEDDMAIFEEDQDLLQCVKSIN

SEQ ID NO: 42 - protein, 477 aa, Papaver somniferum
  source 1..477: note = subspecies: setigerum
MENMKVEVVLKQTIKPSTQTPLHSKTFNLSFLDQHLGPPIYIPFTLYYESGDVNNKNNHCDGYKNNLEEACEHRVSVIKQSLSETLARYYPLAGRMKEDNLAVECNDEGVEYFETRVSDVRLSQVIKRSPNHNSVLRKFLPPCISSCDNSMSIPFDYGFKSKTLLAIQVNIFECGGIVIGMCMAHRLADASTMFTFITDWAATARGAIEDIKGPSFDFSYTLFPQKDVINNFKPFDPMLTREEDLVTKYFVFPASKIVELKRRNVNNIVCQDTSQQNTSPCTRVEAVTSFMWKRYMDSVRAKNQTQATSVEKYGALYTVNLRSRITPPLPANSFGNIYTFTIALSTPSDENDIDDGLRKDVSSPNDLNLVGKVRDAIKKIDDKYTRKLQSSEDELVNDVKPLTSGEAIFLGFSSWCRFPIYEADFGWGKPTWVSIGTMALRNTVFLMDTKSGDGIEAFVNMAKEDMDNFEVKLLADQ

SEQ ID NO: 43 - protein, 485 aa, Papaver somniferum
  source 1..485: note = subspecies: setigerum
MENMKVEVVLEQTIKPSTQTPLHSKTFNLSFLDQHLGPPIYIPFTLYYESGDVNNKNNHCDGYKNNLEEVCEHRVSVIKQSLSETLARYYPLAGRMKEDNLAVECNDEGVEYFETRVSDVRLSQVIKRSPNHNSVLRKFLPPCISSCDNSMSIPFDYGFKSKTLLAIQVNIFECGGIVIGMCMAHRLADASTMFTFITDWAATARGAIEDIKGPSFDFSYTLFPQKDVINNFKPFDPMLTREEDLVTKYFVFPASKIVELKRRNVNNIVCQDTSQQNTSPCTRVEAVTSFMWKRYMDSVRAKNQTQATSVEKYGALYTVNLRSRITPPLPANSFGNIYTFTIALSTPSDENDIDDGLRKDVSSPNDLNLVGKVRDAIKKIDDKYTRKLQSSEDELVNDVKPLTSGEAIFLGFSSWCRFPIYEADFGWGKPTWVSIGTMALRNTVFLMDTKSGDGIEAFVNMAKEDMDNFEVKLLADQLLHVHPTV

SEQ ID NO: 44 - protein, 452 aa, Papaver somniferum
  source 1..452: note = subspecies: setigerum
MSSTVEVISKQTIKPSTPTPIQRKNHSLSLIDQHFAPIYIPIVLFYPAAAVNDTGNVQHGDNTCVLKRSLSETLVHFYPLAGRMKDNIVVDCNDQGVEFTEVKVSGTMCDFLMKPDEQLSGLLPSEAVCMNFVREAQVMIQVNTFDCGSKAISLCVSHKIADASTITTFSRCWAETTIAVSKSTSAVTPIVSSKFHPTFDAASLFPPIKQLISPSGVTPALPELIPSEESKFGKIISKRFLFSATTINSVREKLSALMADKLKYRRLTRVEVVSALIWNSFDKLATTGSVAVMVKHAVNLRKRIDPPLPDVSFGNILEFTKAVVGEAAANTTTQGTVGSSSKLLEELSEFAGQLREPVSKMNKGDHDFDMENTDYEERDLWMSSWCNYGLYDIDFGCGKPVWVTTVATMYPYSDGFFMNDTRCGQGIEVWGNLVEEDMANFQLNLSELLDRI

SEQ ID NO: 45 - protein, 451 aa, Papaver somniferum
MMKVCVSSREKIKPSRPTPGHLKTHKLSFLDQVAARIYVPLLLYYAGNKENVDTDTRCNIIKKSLAETLTKFYILAGKIVNDEIERFVNCNDDGVDFCVTKVSNCQLFQVIKRPDIFDQVTLFLPFDPCDNEITASGDFLLSVQVNVFEDCRGMVIGLCINHKVADASSITTFVNYWATIARGLVLNVDDRQIQDPCFQVQSIFPQKEKGIGFKISSSSIDGTLVTKKFGFEASKLAELKERCKFTTEPEDGYKPTRVEALSAFLWKCFIDIDQAKLKGVARTKVYLATNAVNMRSRMVPQLPTSSFGNIISITDAVFSINNDDSTGINDPYYPKLVRKFRDAIKKIDRDYIEALRSTDLLLNNMMKLIEHVLSGHTLSIYFSSWCRFPLYETDFGWGKPIWVSTCTIPQKNVIVLMDSNSSADGIEAYVTLAKEDMGELEHHEELLALIS

SEQ ID NO: 46 - protein, 232 aa, Papaver somniferum
MGAMKFFSFLAVAMVLSLAHIQAQQGNWGDETVPYTMGPEKITKLRFYFHDIVTGNNPTAVQIAQATGTNSSSTLFGALFMIDDPLTEGPDPDSRLVGRAQGFYGSAGQNEAALILGMSLVFTGNEKFNGSTISVLSRNPVTHTEREFAIVGGTGYFQFARGFISAKTYSLVGPNAVVEYNCTIVHPSSVSESGKSNSSPGKSDSNSGSQISLGSNLVFVSVIAYVTIILSL

SEQ ID NO: 47 - protein, 224 aa, Papaver somniferum
  source 1..224: note = subspecies: setigerum
MVLSMSHSQAQEGNWGDESVPYTMGPEKMTKLRFYFHDIITGNSPTAVQIAQATGTNTSATMFGALMMIDDPLTEGPDPNSRLVGRAQGFYGSAGQNELALILGMSLVFTGNEKFNGSTISVLSRNPVMHTEREFAIVGGTGYFQFARGFISAKTYSLVGPNAVVEYNCTIVHPSSVSESGKSDSSSGKSDSSSGSQISLGTNLVFLSVIAFVTIIVSPQHFSW

SEQ ID NO: 48 - protein, 210 aa, Papaver somniferum
MTKTVLVDDIPFPQNITTVTTEKQLPLLGQGITDMEIHFLQIKFTAIGTAIGVYLEPEIASHLQQWKGKTGAELSQDDEFFAAVVSASVEKYVRVVVIKEIKGSQYMLQLESWVRDELAAADKYEDEEEESLDKVIEFFQSKYLKQLSFIPSHFSATTPAVAEIGLEIEGQKDLKIKVENGNVIEMIQKWYLGGTRGVSPSTTQSLATSL

SEQ ID NO: 49 - protein, 210 aa, Papaver somniferum
MPFLKAIEIEGCKFRPFVTPPGSTQILFLAGSGVKEEFGDSKSMKYSSCAIYLQPTCILYLAKAWAQKSVVDITQSLNFFMDIATGPFEKYCRITMLETAKGEDYAAMITKNCEEMLTNSKRYSETAKAALTKFSEAFNGRTLASGSSIHVTVSTSNSVTLAFTEDGSTPKQGDVTLDCKEVGEAFLMSTISLHTTIRESMGSRISGLYK

SEQ ID NO: 50 - protein, 166 aa, Papaver somniferum
  source 1..166: note = subspecies: setigerum
MAPMAQLSEIQVEQFVFPPTMTPPSSTESLFLGGAGVRGLQIQDRFIKFTAIGVYLAEEAIPSLSPKWKSKSPEELTDDVEFFMDIVTGPFEKFVKITMILPLTGDQYAEKVTENCIQYLKSKDMYTDAEAKAVERFIEIFKNEMFPPASSILFTISPAGSLTVGF

SEQ ID NO: 51 - protein, 160 aa, Papaver rhoeas
MVYLEPEIATHLKQWKGKTGAELSQDDDFFSAVVSAPVEKYVRVVVIKEIKGSQYMLQLESWVRDELAAADKYEDEEEESLDKVIEFFQSKYLKQHSVIITFHFSATTPAVAEIGLEIEGQKDLKIKVENGNVVEMIQKWYLGGTRGVSPSTTQSLATSL

SEQ ID NO: 52 - protein, 206 aa, Papaver bracteatum
MTKMVLVDDIPFPQNITTATTAKQLPLLGQGITDMEIHFLQIKFTAIGVYLEPEIASHLKQWKGKTGAELSQDDEFFSAIVSAPVEKYVRVVVIKEIKGSQYMLQLESWVRDELAAADKYEDEEEESLEKVIEFFQSKYLKQHSVIPFHFSATTPAVAEIGLEIEGHKDLKMKVENGNVVEMIQKWYLAGTRGVSPSTTQSLATSL

SEQ ID NO: 53 - protein, 216 aa, Papaver bracteatum
MAPMAQLSEIQVEQFVFPPTMTPPSSTESLFLGGAGVRGLQIQDRFIKFTAIGVYLAEEAIPSLSPKWKSKTPEELTNDVEFFMDIVTGPFEKFVKITMILPLTGDQYAEKVTENCVEYLKSKDLYTDAEAKAVERFIEIFKNEMFPPASSILFTISPTGSLTVGFSKDTSIPEARNAVIENKALSESILESIIGKNGVSPAAKQSLAERISELLK

SEQ ID NO: 54 - protein, 151 aa, Panax ginseng
MGLTGKLICQTGIKSDGDVFHELFGTRPHHVPNITPANIQGCDLHEGEFGKVGSVVIWNYSIDGNAMIAKEEIVAIDEEDKSVTFKVVEGHLFEEFKSIVFSVHVDTKGEDNLVTWSIDYEKLNESVKDPTSYLDFLLSVTRDIEAHHLPK

SEQ ID NO: 55 - protein, 157 aa, Arachis hypogaea
MGVFTFEDEITSTVPPAKLYNAMKDADSITPKIIDDVKSVEIVEGNGGPGTIKKLTIVEDGETKFILHKVESIDEANYAYNYSVVGGVALPPTAEKITFETKLVEGPNGGSIGKLTLKYHTKGDAKPDEEELKKGKAKGEGLFRAIEGYVLANPTQY

SEQ ID NO: 56 - protein, 166 aa, Hypericum perforatum
MGIDPFTMAAYTIVKEEESPIAPHRLFKALVLERHQVLVKAQPHVFKSGEIIEGDGGVGTVTKITFVDGHPLTYMLHKFDEIDAANFYCKYTLFEGDVLRDNIEKVVYEVKLEAVGGGSKGKITVTYHPKPGCTVNEEEVKIGEKKAYEFYKQVEEYLAANPEVFA

SEQ ID NO: 57 - protein, 158 aa, Lupinus luteus
MGVFTFQDEYTSTIAPAKLYKALVTDADIIIPKAVETIQSVEIVEGNGGPGTIKKLTFIEGGESKYVLHKIEAIDEANLGYNYSIVGGVGLPDTIEKISFETKLVEGANGGSIGKVTIKIETKGDAQPNEEEGKAAKARGDAFFKAIESYLSAHPDYN

SEQ ID NO: 58 - protein, 161 aa, Fragaria X ananassa
MAGVFTYETEFTSVIPPPRLFKAFILDADNLIPKIAPQAVKCAEIIEGDGGVGTIKKITFGEGSQFGSVTHKIDGIDKENFVYSYSLIEGDALSDKIEKISYETKLVSSSDGGSIIKSTSNYHTKGDVEIKEEHVKAGKEKFSHLFKLVEGYLLANPNEYC

SEQ ID NO: 59 - protein, 150 aa, Actinidia chinensis
  source 1..150: note = subspecies: deliciosa
MDLSGKMVKQVEILSDGIVFYEIFRYRLYLISEMSPVNIQGVDLLEGNWGTVGSVIFFKYTIDGKEKTAKDIVEAIDEETKSVTFKIVEGDLMELYKTFIIIVQVDTKGEHNSVTWTFHYEKLKEDVEEPNTLMNFCIEITKDIETYHLK

SEQ ID NO: 60 - protein, 164 aa, Thalictrum flavum
MGIINQVSTVTKVIHHELEVAASADDIWTVYSWPGLAKHLPDLLPGAFEKLEIIGDGGVGTILDMTFVPGEFPHEYKEKFILVDNEHRLKKVQMIEGGYLDLGVTYYMDTIHVVPTGKDSCVIKSSTEYHVKPEFVKIVEPLITTGPLAAMADAISKLVLEHKS

SEQ ID NO: 61 - protein, 155 aa, Vigna radiata
MVKEFNTQTELSVRLEALWAVLSKDFITVVPKVLPHIVKDVQLIEGDGGVGTILIFNFLPEVSPSYQREEITEFDESSHEIGLQVIEGGYLSQGLSYYKTTFKLSEIEEDKTLVNVKISYDHDSDIEEKVTPTKTSQSTLMYLRRLERYLSNGSA

SEQ ID NO: 62 - protein, 364 aa, Papaver sp.
MEKAKLMKLGNGMEIPSVQELAKLTLAEIPSRYVCANENLLLPMGASVINDHETIPVIDIENLLSPEPIIGKLELDRLHFACKEWGFFQVVNHGVDASLVDSVKSEIQGFFNLSMDEKTKYEQEDGDVEGFGQGFIESEDQTLDWADIFMMFTLPLHLRKPHLFSKLPVPLRETIESYSSEMKKLSMVLFNKMEKALQVQAAEIKGMSEVFIDGTQAMRMNYYPPCPQPNLAIGLTSHSDFGGLTILLQINEVEGLQIKREGTWISVKPLPNAFVVNVGDILEIMTNGIYHSVDHRAVVNSTNERLSIATFHDPSLESVIGPISSLITPETPALFKSGSTYGDLVEECKTRKLDGKSFLDSMRI

SEQ ID NO: 63 - protein, 360 aa, Papaver sp.
METPILIKLGNGLSIPSVQELAKLTLAEIPSRYTCTGESPLNNIGASVTDDETVPVIDLQNLLSPEPVVGKLELDKLHSACKEWGFFQLVNHGVDALLMDNIKSEIKGFFNLPMNEKTKYGQQDGDFEGFGQPYIESEDQRLDWTEVFSMLSLPLHLRKPHLFPELPLPFRETLESYLSKMKKLSTVVFEMLEKSLQLVEIKGMTDLFEDGLQTMRMNYYPPCPRPELVLGLTSHSDFSGLTILLQLNEVEGLQIRKEERWISIKPLPDAFIVNVGDILEIMTNGIYRSVEHRAVVNSTKERLSIATFHDSKLESEIGPISSLVTPETPALFKRGRYEDILKENLSRKLDGKSFLDYMRM

SEQ ID NO: 64 - protein, 364 aa, Papaver somniferum
MEKAKLMKLGNGLSIPSVQELAELTFAEVPSRYVCTNDENLLLMTMGASEIDDETVPVIDLQNLLSPEPAIGKSELDWLHYSCKEWGFFQLVNHGVDALLVDHVKSEIHSFFNLPLNEKTKYGQRDGDVEGFGQAFLVSENQKLDWADMFFINTLPLHLRKPHLFPNLPLPLRETIESYSSEMKKLSMVLFEMMGKAIEVIDIKEAITEMFEDGMQSMRMNYYPPCPQPERVIGITPHSDFDGLTILLQLNEVEGLQIRKEDKWISIKPLPDAFIVNVGDIWEIMTNGVHRSVDHRGVINSTKERLSIATFHSPKLELEIGPISSLIRPETPAVFKSAGRFEDLLKEGLSRKLDGKSFLDCMRM

SEQ ID NO: 65 - protein, 294 aa, Papaver somniferum
MEKAKLMKLGNGMEIPSVQELAKLTLAEIPSRYVCANENLLLPMGASVINDHETIPVIDIENLLSPEPIIGKLELDRLHFACKEWGFFQVVNHGVDASLVDSVKSEIQGFFNLSMDEKTKYEQEDGDVEGFGQGFIESEDQTLDWADIFMMFTLPLHLRKPHLFSKLPVPLRETIESYSSEMKKLSMVLFNKMEKALQVQAAEIKGMSEVFIDGTQAMRMNYYPPCPQPNLAIGLTSHSDFGGLTILLQINEVEGLQIKREGTWISVKPLPNAFVVNVGDILEIMTNGIYHSVD

SEQ ID NO: 66 - protein, 364 aa, Papaver somniferum
METAKLMKLGNGMSIPSVQELAKLTLAEIPSRYICTVENLQLPVGASVIDDHETVPVIDIENLISSEPVTEKLELDRLHSACKEWGFFQVVNHGVDTSLVDNVKSDIQGFFNLSMNEKIKYGQKDGDVEGFGQAFVASEDQTLDWADIFMILTLPLHLRKPHLFSKLPLPLRETIESYSSEMKKLSMVLFEKMEKALQVQAVEIKEISEVFKDMTQVMRMNYYPPCPQPELAIGLTPHSDFGGLTILLQLNEVEGLQIKNEGRWISVKPLPNAFVVNVGDVLEIMTNGMYRSVDHRAVVNSTKERLSIATFHDPNLESEIGPISSLITPNTPALFRSGSTYGELVEEFHSRKLDGKSFLDSMRM

SEQ ID NO: 67 - protein, 369 aa, Papaver bracteatum
METPKSIKLGGSLLVPSVQELAQQSFAEVPARYVRDDLEPLTDLSGVSMIDQTIPVIDLQKLQSPVPIIRELESEKLHSACKEWGFFQVVNHGVDILLVEKTKSEIKDFFNLPMDEKKKFWQEEGDIQGFGQAFVQSEDQKLDWADIFLMVTLPRHTRNPRLFPKLPLPLRNTMDSYSSKLSKLASTLIEMMGKALHMETSVLAELFEDGRQTMRINYYPPCPQPKDVIGLTPHSDGGGLTILLQLNEVDGLQIRKEKIWIPIKPLPNAFVVNIGNILEIMTNGIYRSVEHRATIHSTKERLSVAAFHNPKVGVEIGPIVSMITPESPALFRTIEYDDYGKKYFSRKLDGKSSLDFMRIGEGDEENKAT

SEQ ID NO: 68 - protein, 366 aa, Papaver bracteatum
METPKLIKLGGSLLVPSVLELTKQSPAEVPARYIRNDLEPMTDLSSASLTDQTIPVIDLQNLLSPEPELELEKLHSGCKEWGFFQVMNHGVDILLVEKVKSEIQGFFNLPIDEKNKFWQEEGDLEGYGKAFVHSEDEKLDWADMFFILTQPQYMRKPRVFPKLPLRLRETIESYSLELSKLGLTLLDLMGKALQIETGVMSELFEDGRQTMRMNYYPPCPQPEHVIGLTPHSDGGALTILLQLNQVDGLQIRKEEIWVPIKPLPNAFVVNIGDILEIMSNGVYRSVEHRATINSSKERLSVAIFQSPKHGTEIGPILSMITPEAPALFKTIPYEDYLRKFFSRKLGGKSFVDSMRIGESDEDNNTA

SEQ ID NO: 69 - protein, 366 aa, Papaver bracteatum
METQKQENFGASLSVPNVQELAKQSPEQVPDRYIRSDQDSSTNISCPSMTDQIPVIDLQSLLSPDPIIGELELERLHSACKEWGFFQVVNHGVDNLLVEKVKSEIQGFFNLPMDEKKKFWQEEGDFEGFGQAFVFSEDQKLDWGDVFFILTQPQHMRKPRLFPKLPLPFRKTIESYSLETNKLSMTLLELMEKALKIETGVMTELFEGGIQRMRMTYYPPCPQPKHVIGLTPHSDPDALTILLQLNEVDGLQIRKEKIWVPIKPLSNAFVVNIGDILEIMSNGIYRSVEHRATVNSTKERLSVATFHSPRKDTEIGPILITPETPALFRTSGFEDYFRKFFAHKLNGKSFLSSIRIGETDEGNNAT

SEQ ID NO: 70 - protein, 379 aa, Papaver bracteatum
MEAPKLIMLGGSLFVPSVQELAKQSLAEVPVRYVRDDQDTLGNNINITPMSMIDQSIPVIDLEKLLSPEPIVGELELERLHSACKEWGFFQVVNHGVDSLLVEKVKSEIEGFFKLPMDEKTKFWQEEGDIEGFGQVFVHSQDQKLDWGDMFLMQTLPRHTRKPRLFPNLPLPLRQTIESYSSELSKLVLTLVDLMGKALQMESGVLTELFENGIQRMRMNYYPPCPQPEQVIGLTPHSDVGGLTILLQLNEVDGLQIKKDKVWVPIKPLANAFVVNVGDALEIMSNGIYRSVEHRATINSTKERLSIATFHNPRADREIGPIPSMISPETPALFKTTGYEEYFKKFFSRKLEGKSFLDSLRIREGDEHCGRLDVKGPCN

SEQ ID NO: 71 - protein, 365 aa, Papaver bracteatum
MEIPNPIKIGSSLLVPSVQELAKQSFAEVPARYIRNDVDPLITKLSDVSLIDQTVPVIDLQKLLSPEPIVGELELERLHSACKEWGFFQVVNHGVDNLLVEKVKSEIQGFFNLPMEEKKKFWQEEGDFEGFGQMFVQSEEQKLDWGDMFFILTQPQHMRKPRLFSKLPLPLRETIESYSLELIKLGLTIIKLMEKALQIDAGVMAELFEDGIHTMRMNYYPPCPQPEHVIGLTPHSDGGGLTILLQLNEVDGLQIRRENIWVPIKPLPNAFVVNIGDILEILSNGIYRSVEHRSTVNATKERLSVATFQNPKQESVIGPNMITPERPALFRKIVYKDYMKKLFSRKLDGKSFLDSLRIGEGDERP

SEQ ID NO: 72 - protein, 349 aa, Papaver bracteatum
METLKTVKPGGSLFIPNGQELAKQSLEEVYVGNDQDTMLLIGQTIPVIDLQKLLSPEPITGDMELDKLHSACKEWGFFQVVNHGVDILLVEKVKSEVHDFFNIPMDEKKPFWQEEGDLEGFGQVFITSEDQQLDWGDMFFMVTLPKHMRKPRLFLKLPLPLRETIESYSLKLSKLGVTLVELMGKALQMEDRIMSELFDDGRQTMRMNYYPPCPQPEQVIGLTPHSDPGGLTILLELNEVNGLIRKENIWVPIIPLPNAFIVNIGDILEIMSNGIYHSVEHRATINSTKERLSVAMFNSPKVDTEIGPIHSMITPETPALFRTIGYDEYLKIFFSRKLDGKSLLESMKI

SEQ ID NO: 73 - protein, 379 aa, Papaver bracteatum
  VARIANT 373: note = Any amino acid
  VARIANT 377: note = Any amino acid
MEAPKLIMLGGSLFVPSVQELAKQSLAEVPVRYVRDDQDTLGNNINITPMSMIDQSIPVIDLEKLLSPEPIVGELELERLHSACKEWGFFQVVNHGVDSLLVEKVKSEIEGFFELPVDEKKKFWQEEGDIEGFGQIFVHSEDQKLDWADMFYMLTLPPNMRKPRLFPNLPLPLRQTIDSYSSELSKLVLTLVDLMGKALQMESGVLTELFENGIQRMRMNYYPPCPQPEQVIGLTPHSDVGGLTILLQLNEVDGLQIKKDKIWVPIKPLRNAFVVNVGDALEIMSNGIYRSVEHRATINSTKERLSIATFHNPRADREIGPIPSMISPETPALFKTTGYEEYFKKFFSRKLEGKSFLDSLRIGEGDEHCGRLXVKGXCN

SEQ ID NO: 74 - protein, 362 aa, Papaver bracteatum
METPKLMKLGGSLFVPSVQELAKQSLAEVPARYVRDDRDMVGNIINVTPMSMIDQSIPVIDLEKLLSPDLIVGELELERLHSACKEWGFFQVVNHGVDSLLVEKVKSEIEGFFELPMDEKKKFWQEEGDAEGFAQFFVQSEDQKLDYSGDMFFMLNLPQHMRKPRLFLKLPLPLRETIESYSLKLSKLGVTLVELMGKALQMEDRIMSELFDDGRQTMRMNYYPPCPQPEQVIGLTPHSDPGGLTILLELNEVNGLIRKENIWVPIIPLPNAFIVNIGDILEIMSNGIYHSVEHRATINSTKERLSVAMFNSPKVDTEIGPIHSMITPETPALFRTIGYDEYLKIFFSRKLDGKSLLESMKI

SEQ ID NO: 75 - protein, 365 aa, Papaver bracteatum
METPKLRDFGSFLPVPSVQELAKQVLTEIPPRYIRTDLEALNKLSCASNTDQTVPIIDMQCLLSAEPEMELEKLHSACKEWGFFRVVNHGVDNLESVKSEIESFLNLPVNAKNKYGQKQGDDQGFGSRFVLSEEQKLDWGDFFYMVTRPLYLRKPHLFPELPLPLRETIESYSSEVSKLAMALFEMMGKALKIETGVMTEIFEGGMQAMRMNYYPPCPRPDLVIGLNAHSDFGGLTILLQLNEVEGLEIRNKGEWVSVKPLANAFVVNVGDVMEILTNGIYHSVEHRATINSSKERLSVATFHYPKLETGIGPLPCMITPKTPALFGRIERYELLLRKYYARKLNGKSTLDCMRIGNGFEDDNTA

SEQ ID NO: 76 - protein, 370 aa, Papaver bracteatum
MEAPKLIMLGGSLFVPSVQELAKQSLAEVPARYVRDDQDTLGNNINITPMSMIDQSIPVIDLEKLLSPEPIVGELELERLHSACKEWGFFQVVNHGVDSLLVEKVKSEIEGFFELPVDEKKKFWQEEGDIEGFGQIFVHSEDQKLDWADMFYMLTLPPNMRKPRLFPNLPLPLRQTIDSYSSELSKLVLTLVDLMGKALQMESGVLTELFENGIQRMRMNYYPPCPQPEQVIGLTPHSEVGGLTILLQLNEVDGLQIRKEKIWVPIKPLSNAFIVNIGDILEIMSNGIYRSVEHRATVNSTKERLSVATFHSPRKDTEIGPILITPETPALFRTSGFEDYFRKFFAHKLNGKSFLSSIRIGETDEGNNAT

SEQ ID NO: 77 - protein, 313 aa, Papaver bracteatum
MSMIDQSIPVIDLEKLLSPEPIVGELELERLHSACKEWGFFQVVNHGVDSLLVEKVKSEIEGFFELPVDEKKKFWQEEGDIEGFGQIFVHSEDQKLDWADMFYMLTLPPNMRKPRLFPNLPLPLRQTIDSYSSELSKLVLTLVDLMGKALQMESGVLTELFENGIQRMRMNYYPPCPQPEQVIGLTPHSDVGGLTILLQLNEVDGLQIRKEKIWVPIKPLSNAFIVNIGDILEIMSNGIYHSVEHRATINSTKERLSVAMFNSPKVDTEIGPIHSMITPETPALFRTIGYDEYLKIFFSRKLDGKSLLESMKI

SEQ ID NO: 78 - protein, 365 aa, Papaver bracteatum
METPKLVKSSGSSLFLSTSVQELAKQSLPEVPARYIRTNLEPLSNVSGDSQSVPVIDLQKLLSSEPIIGELELDKLHSACKEWGFFQVVNHGVDNLVMEKIKTEIQGFFNLSLDEKQKFWKKEGDAEGFGQNFIESEDQKLDWGDTFGMFTLPIHMRNPRLFPELPLPLRETIESYSLDVRKLALALIGLMEKALKIKTSAMSELFEDGGQAMRMNYYPPCPQPEHVIGLTPHSDAGGLTILLQLNEVDGLQIKKDKIWVPIKPLPNAFVVNIGDILEIMTNGIYRSVEHRATINSSKERLSVAAFHSPKGDTLIGPMVSLITPETPALFRTIGYQDYMKKFMSRKLDGKSLVNSMRIGEGDEDK

SEQ ID NO: 79 - protein, 356 aa, Papaver bracteatum
METPTLMKLGNGLSVPSVQELAKATLAEIPSRYICTDENLLTMGASTTDNETVPVIDLQNLLSPEPVIGMLELDRLHSACKEWGFFQLVNHGVDALLVDNEVQGFFNLPMDEKTKYGQKDGDDEGFGQFFVISEDQKLDWADVFYMSTLPLHSRKPHLFPELPLPLRETMESYSSEMKKLSMVLFDMMGKALQVVEIKGITELFEDGAQQIRMNYYPPCPQPELVFGLTSHSDFDGLTILLQLGEVEGLQIKKEERWISIKPLPDAFIVNVGDILEIMTNGIYRSVDHRAVVNSIKERLTIATFHDPRLEAEIGPISSLITPETPALFKRGVFEDLLKEMFLRKLDGKSFLDCMRM

SEQ ID NO: 80 - protein, 350 aa, Papaver rhoeas
GNGLSVPSVQELAKQTLAEIPSRYICTDENPLITGASVVDDETVPVINLQNLLSPEPVIGKLELDKLHSACKEWGFFQVVNHGVNDSLVDSVKSEIEGFFNLPANEKLKYGQKDGDVEGFGQHFVVSEDQKLDWADVFYMVTLPVRLRKPHLFPELPLPLRDTLDSYSSELNKLSMVLLEMMEKALKLVECKGITDFFEDGFQQMRMNYYPPCPRPELVTGLTSHSDFGGLTILLQLNDVEGLQIKKEERWISIKPLPNAFIVNIGDVLEIMSNGIYRSVDHRAVINSTKVRMSVATFHDPRLEAVIGPISSLITPETPALFKRGVFEDLLKEMFLRKLDGKSFLDCMRI

SEQ ID NO: 81 - protein, 351 aa, Papaver somniferum
  source 1..351: note = subspecies: setigerum
LMKLANGMSVPIVQELAKLTVGEIPSRYICTDGNLLTMGASVIDYETVPVIDLQNLQSREPVIEKLELDRLHSACKEWGFFQLLNHGVDASLMDNVRSEIRGFFNLPISDKMKYGQKDGDEEGFGQHFIVSEDQKLDWVDAFMMFTLPLHSRNPRLTPEFPQPLRETVESYSSEMKKLSVLLFELMEKALQVKGITEMFEDGLQSIRMNYYPPCPRPELAIGLTSHSDFDGLTILLQLNEVEGLQIKKEERWISIKPLPNAFIVNVGDVLEVMTNGIYRSVDHRAVVNSTKERLSIATFHDPELESEIGPIASLITPETPALFKRGRFKDLLKENLSTKLDGKSFLDCIRM

SEQ ID NO: 82 - protein, 497 aa, Homo sapiens
MGLEALVPLAVIVAIFLLLVDLMHRRQRWAARYSPGPLPLPGLGNLLHVDFQNTPYCFDQLRRRFGDVFSLQLAWTPVVVLNGLAAVREALVTHGEDTADRPPVPITQILGFGPRSQGVFLARYGPAWREQRRFSVSTLRNLGLGKKSLEQWVTEEAACLCAAFANHSGRPFRPNGLLDKAVSNVIASLTCGRRFEYDDPRFLRLLDLAQEGLKEESGFLREVLNAVPVLLHIPALAGKVLRFQKAFLTQLDELLTEHRMTWDPAQPPRDLTEAFLAEMEKAKGNPESSFNDENLRIVVADLFSAGMVTTSTTLAWGLLLMILHPDVQRRVQQEIDDVIGQVRRPEMGDQAHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVPTGQPRPSHHGVFAFLVTPSPYELCAVPR

SEQ ID NO: 83 - protein, 1049 aa, Bacillus megaterium
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAADEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 84 - protein, 503 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 85 - protein, 502 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSIIFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 86 - protein, 534 aa, Menispermum canadense
MIMMFIDYYSSWLPQTLLLQSILLAVSLVIFINLFLTRRRSYSSKSHTNIIHPPKAAGALPVIGHLYTLFRGLSAGVPLYRQLDAMADRYGPAFIIHLGVYPTLVVTCRELAKECFTTNDQTFATRPSTCAGKYIGYNYAFFGFAPYGPYWREARKIATVELLSNYRLDSLRHVREAEVGRNVDELYALHASSSTNKQNMMKIDMKQWFDQVTLNVILMMVVGKRCVTTGGNEEEVRVVKVLHEFFKHLGTLSVSDVVPYVEWMDLDGNIGRMKSTAKELDCILGRWLEEHRRERRSDFMDAMLAMVEGIKIPYYDSDTVIKAICLNLLNAGSDTLGITMTWALSLLLNNRHVLKKVKDELDVHVGKNRQVEELDVKNLVYLHAVVKETLRLFPPAPLGVPHEAMEDCVVGGFHVAKGTRLVVNVWKLHRDPSVWSDPLAFKPERFLDNNTVDVRGQHFQLLPFGSGRRGCPGITFALQVAHLTLARLLHGFEWDTPDGAPVDMSEVSVLTTAKKNPVEVLFTPRLPAEVYTQN

SEQ ID NO: 87 - protein, 527 aa, Nigella sativa
MLSIHDSTMVFLQLQAICGIFGFIFIITWWTRWKSSNKMKAPEVAGAWPVIGHLHLLGGGRPLYQLLGDMSDKYGPAFTLRMGIQKALVVSSWEVAKECLTTNDRALATRPSSAGGKYMGYNNALIPFSPYGPYWRDMRKIATLELLSNHRLEELKHVREMEINTCISDMYKLCQVEDGVEIKPISVDLSQWFADLTFNVVVMMITGKRYIGSTDAGDMNEIRHFQAALVKFMRLLRISLLVDVFPVLQWINYGGFKGVMKSTARDIDSVLENWLQEHQRKRLSPDFNGNHDFIDVMISTLEGTEFSDYDHNTIIKAISMAMVVGGTDTTTTTLIWAISLLLNNPNAMKKVQEELEIHVGKERNVDGSDIQHLVYLQAVVKETLRLYPPVPLSVMHQAMEDCVIGSYNIQAGTRVLFNLWKLHRDSSVWSDPLEFRPERFLTSHVDVDVRGQHFELIPFGSGRRSCPGISFALQVIHLTIARLFHGFNLTTPGNSSVDMSEISGATLSKVTPLEVLVTPRLSSKLYN

SEQ ID NO: 88 - protein, 525 aa, Hydrastis canadensis
MDSLLQLQIIGALAALIFTYKLLKVICRSPMTDGMEAPEPPGAWPIIGHLHLLGGQDPIARTLGVMTDKYGPILKLRLGVHTGLVVSNWELAKECFTTNDRVLASRPMGAAGKYLGYNYAIFGLAPHGPYWSEVRKIVLRELLSNQSLEKLKHVRISEINTCLKNLFSLNNGNTPIKVDMKQWFERPMFNVVTMMIAGKRYFSMENDNEAMNFRKVATEFMYLTGVFVVSDALPYLEWLDLQGHVSAMKRTAKELDIHVGKWLEEHRRAKLLGETKNEDDFVDVLLTILPEDLKDNQTYIHDRDTIIKATALALFLAASDTTAITLTWALSLILNNPDVLKRAQDELDKHVGKEKLVKESDIINLVYLQAIIKETLRLYPAAPLLLPHEAMEDCTVGGYHVPKGTRIFVNIWKLQRDPRVWFDPNEFRPERFLTTHANVDFKGQHFEYIPFSSGRRVCPGITFSTQIMHLTLAHLLHEFNIVTPTKSNAGVDMTESLGITMPKATPLEVLLTPRLPSNLYNQYRD

SEQ ID NO: 89 - protein, 550 aa, Eschscholzia californica
MNLLIFFQFLLQFQVLVGLSVLLAFSYYLWVSKNPKINKFKGKGALLAPQAAGAWPIVGHLPQLVGPKPLFRILGAMADNYGPIFMLRFGVHPTVVVSSWEMTKECFTTNDRHLASRPSNAASQYLIYEVYALFGFSLYGSSYWRDARKIATLELLSHRRLELLKHVPYTEIDTCIKQLHRLWTKNNKNQNNPELKVEMNQFFTDLTMNVILKLVVGKRFFNVDDAADHEKEEARKIQGTIFEFFKLTEGSVSAGALPLLNWLDLNGQKRAMKRTAKKMDSIAEKLLDEHRQKRLSKEGVKGTHDHNDFMDVLLSILDADQGDYSHHPFNYSRDHVIKATTLSMILSSMSISVSLSWALSLLLNNRHVLKKAQDELDMNVGKDRQVEEGDIKNLVYLQAIVKETFRMYPANPLLLPHEAIEDCKIGGFNVPAGTRVVVNAWKLQHDPRVWSNPSEFKPERFLNDQAAKVVDVRGQNFEYLPFGSGRRVCPGISFSLQTIHMSLARLVQAFELGTPSNERIDMTEGSGLTMPKTTPLHVLLNPRLPLPLYE

SEQ ID NO: 90 - protein, 537 aa, Glaucium flavum
MELINSLEIQPITISILALLTVSILLYKIIWNHGSRKNNKSNKNNRKTSSSAGVVEIPGAWPIIGHLHLFNGSEQMFHKLGSLADQYGPAPFFIRFGSRKYVVVSNWELVKTCFTAQSQIFVSRPPMLAMNILFFPKDSLSYIQHGDHWRELRKISSTKLLSSHRVETQKHLIASEVDYCFKQLYKLSNNGEFTLVRLNTWCEDMALNVHVRMIAGMKNYVAAPGSGEYGGQARRYRKALEEALDLLNQFTITDVVPWLGWLDHFRDVVGRMKRCGAELDSIFATWVEEHRVKRASGKGGDVEPDFIDLCWESMEQLPGNDPATVIKLMCKEHIFNGSGTSSLTLAWILSLIMNNPYVIKKAREELEKHVGNHRQVEESDLPNLLYIQAIIKEGMRLYTPGPFIDRNTTEDYEINGVHIPAGTCLYVNLWKIHRDPNVYEDPLEFKPERFLKNNSDLDLKGQNYQLLPFGAGRRICPGVSLALPLMYLTVSRLIHGFDMKLPKGVEKADMTAHGGVINQRAYPLEVLLKPRLTFQQA

SEQ ID NO: 91 - protein, 568 aa, Stylophorum diphyllum
MTIGALALLSFIYFLRVSVIKRTKYTNTAVTATNKLENDEDEANHSKRVVAPPEVAGAWPILGHLPQLVGLKQPLFRVLGDMADKYGPIFIVRFGMYPTLVVSSWEMAKECFTTNDRVLASRPASASGKYLTYNYAMFGFTNGPYWREIRKISMLELLSHRRVELLKHVPSTEIDSSIKQLYHLWVENQNQNKQGDHQVKVDMSQLLRDLTLNIVLKLVVGKRLFNNNDMDHEQDEAARKLQKTMVELIKVAGASVASDALPFLGWLDVDGLKRTMKRIAKEIDVIAERWLQEHRQKKLTSNDKGGSNNIQGGGGDNDFMDVMLSILDDDSNFFINYNRDTVIKATSLTMILAGSDTTTLSLTWALTLLATNPGALRKAQDELDTKVGRDRQVDERDIKNLVYLQAIVKETLRMYPAAPLAIPHEATQDCIVGGYHVTAGTRVWVNLWKLQRDPHAWPNPSEFRPERFLAVENDCKQQGTCDGEAANMDFRGQHFEYMPFGSGRRMCPGINFAIQIIHMTLARLLHSFELRVPEEEVIDMAEDSGLTISKVTPLELLLTPRLPLPLYI

SEQ ID NO: 92 - protein, 561 aa, Stylophorum diphyllum
FCQFQGIVGILLAFLTFLYYLWRASITGLRTKPKHNDFKVTKAAPEADGAWPIVGHFAQFIGPRPLFRILGDMADKYGSIFMVRFGMYPTLVVSSWEMAKECFTTNDRFLASRPASAAGKYLTYDFAMLSFSFYGPYWREIRKISMLELLSHRRVELLKHVPSTEIDSSIKQLYHLWVENQNQNKQGDHQVKVDMSQLLRDLTLNIVLKLVVGKRLFNNNDMDHEQDEAARKLQKTMVELIKVAGASVASDALPFLGWLDVDGLKRTMKRIAKEIDVIAERWLQEHRQKKLTSNDKGGSNNIQGGGGDNDFMDVMLSILDDDSNFFINYNRDTVIKATSLTMILAGSDTTTLSLTWALTLLATYPLCALRKAQDELDTKVGRDRQVDERDIKNLVYLQAIVKETLRMYPAAPLAIPHEATQDCIVGGYHVTAGTRVWVNLWKLQRDPHAWPNPSEFRPERFLAVENDCKQQGTCDGEAANMDFRGQHFEYMPFGSGRRMCPGINFAIQIIHMTLARLLHSFELRVPEEEVIDMAEDSGLTISKVTPLELLLTPRLPLPLYI

SEQ ID NO: 93 - protein, 561 aa, Chelidonium majus
MDLFIFFSRFQYIVGLLAFLTFFYYLWRVSITGTRIKTNQNIMNGTNMMAPEAAGAWPIVGHLPQLVGPQPLFKILGDMADKYGSIFMVRFGMHPTLVVSSWEMAKECFTTNDKFLASRPTSAGGKYLTYDFAMFGFSFYGPYWREIRKISTLELLSHRRVELLKHVPYTEIGGSIKQLYKLWMETQNQNKQRDDHQVKVDMSQVFGYLTLNTVLKLVVGKGLFNNNDMNHEQEEGRKLHETVLEFFKLAGVSVASDALPFLGWLDVDGQKRSMKRIAKEMDLIAERWLQEHRQKRLTSNNKASSGHDDFMSVLLSILDDDSNFFNYNRDTVIKATSLNLILAASDTTSVSLTWVLSLLVTNPGALKKVQDELDTKVGRNRHVEERDIEKLVYLQATVKETLRMYPAGPLSVPHEATQDCTVGGYQVTAGTRLVVNVWKLQRDPRVWPNPSEFKPERFLPDGCEVGCGEAANMDFRGQHFEYIPFGSGRRMCPGIDFAIQIIHMTLACLLHAFEFQVPSSLDKHLVPAVIDMSEGSGLTMPKVTPLEVLLNPRLPLPLYEL

SEQ ID NO: 94 - protein, 561 aa, Eschscholzia californica
MEKPILLQLQPGILGLLALMCFLYYVIKVSLSTRNCNQLVRHPPEAAGSWPIVGHLPQLVGSGKPLFRVLGDMADKFGPIFMVRFGVHPTLVVSSWEMAKECFTSNDKFLASRPPSAASIYMAYDHAMLGFSSYGPYWREIRKISTLHLLSHRRLELLKHVPHLEIHNFIKGLYGIWKDHQKQQQQPTARDDQDSVMLEMSQLFGYLTLNIVLSLVVGKRVCNYHADGHLDDGEEAGQGQKLHQTITDFFKLSGVSVASDALPFLGLFDLDGQKKIMKRVAKEMDFVAERWLQDKKSSLLLSSKSNNKQNEAGEGDVDDFMDVLMSTLPDDDDSFFTKYSRDTVIKANSLSMVVAGSDTTSVSLTWALSLLLNNIQVLRKAQDELDTKVGRDRHVEEKDIDNLVYLQAIVKETLRMYPAGPLSVPHEAIEDCNVGGYHIKTGTRLLVNIWKLQRDPRVWSNPSEFRPERFLDNQSNGTLLDFRGQHFEYIPFGSGRRMCPGVNLATPILHMTLARLLQSFDLTTPSSSPVDMTEGSGLTMPKVTPLKVLLTPRLPLPLYDY

SEQ ID NO: 95 - protein, 567 aa, Papaver bracteatum
MDVAIIVDHHYLQPFVSIAGLLALLSFFYCIWVFIIRPRIIKSNLDERKLSPSSPPEVAGAWPIVGHLPQLIGSTPLFKILADMSNKYGPIFMVRFGMYPTLVVSSWEMSKECFTTNDRLFATRPPSAAGKYLTKALFAFSVYGPYWREIRKISTIHLLSLRRLELLKHGRYLEIDKCMKRLFEYWMEHHKNIISTTSSVKVNMSQVFAELSLNVVLKIIVGKTLFIKNGNEDYTKEEEEGQKLHKTILKFMELAGVSVASDVLPFLGWLDVDGQKKQMKRVYKEMNLIASKWLGEHRERKRLQIIQKRGAARGSNYDDGNDFMDVLMSILDEENDDLFFGYSRDTVIKSTCLQLIVAASDTTSLAMTWALSLLLTNPNVLQKAQDELDTKVGRDRIIEEHDIECLVYLQAIVKETLRLYPPAPLSLPHEAMEDCTVGGYQVKAGTRLVVNLWKLQRDPRVWSNPLEFKPERFLPQSDGGFGGEEARMDFRGQHFEYTPFGSGRRICPGIDFFLQTVHMALARLLQAFDFNTAGGLVIDMVEGPGLTMPKVTPLEVHLNPRLPVTLY

SEQ ID NO: 96 - protein, 558 aa, Papaver bracteatum
MQVDWPNILQKYYPIITCSLLTLLSFYYIWVSITKPSRNSKTKLPPPEVAGSWPIVGHLPQLVGSTPLFKILANMSDKYGPIFMVRFGMHPTLVVSSWEMSKECFTTNDKFLASRPPSASAKYLGYDNAMFVFSDYGPYWREIRKISTLQLLTHKRLDSLKNIPYLEINSCVKTLYTRWAKTQSQIKQNVGGAADDFVKVDMTEMFGHLNLNVVLRLVVGKPIFIQKDNADEDYTKDGHNKEELGQKLHKTIIEFFELAGASVASDVLPYLGWLDVDGQKKRMKKIAMEMDLFAQKWLEEHRQKGINHDNENDFMAVLISVLGEGKDDHIFGYSRDTVIKATCLTLIVAATDTTLVSLTWALSLLLTNPRVLSKAQDELDTVVGKERNVEDRDVNHLVYLQAVIKETLRLYPPSPLAVPHEAIENCNVGGYEVKARTRLLVNLWKIHRDPRVWSNPLEFKPERFLPKLDGGTGEASKLDFKGQDFVYTPFGSGRRMCPGINFASQTLHMTLARLLHAFDFDIESNGLVIDMTEGSGLTMPKVTPLQVHLRPRLPATLY

SEQ ID NO: 97 - protein, 571 aa, Papaver bracteatum
MMDLAMFIDQYFSLAKIAGLLALLSFFYYLWISTLWSPRNPKLSSVSPPEVAGAWPILGHLPQLLGSRPLFKILADMSDNYGPIFMVRFGMHPTLVVSSWEMAKECFTTNDRFLAGRPSGAANKYLTFALFGFSTYGPYWREIRKIATLHLLSHRRLELLKHVPDLEVTNCMKHLHRRWIDSQNQIKQNDAAAGSVKVDMGRVFGELTLNVVLKLVAGKSIFFKNDNTRQYDSKDGHNKEEEEGKKLHKTIIDFYSLAGASVASDVLPFLGWLDVDGQKKRMKRVAKDMDFIAAKWLEEHRHQKRQTVLSSSATLGSSNHDDAKDFMDVLMSILDGENDDLFFGYSRDTVIKTTCLQLIAAAADTTSVTMTWALALLITNPTILRKAQDELDTKVGKDRNIEERDINDLVYLQAIVKETLRMYPAGPLNVPHEAIADCNIGGYEVRAGTRLLVNLWKMHRDPRVWSNPSEFKPERFLPQLDGGSGGEAANLDFRGQDFEYLPFSAGRRMCPGIDFSLQTLHMTLARLLHGFDFNNDSAGIIIDMEEGSGLTMPKLTPLEIYLCPRLPAKLY

SEQ ID NO: 98 - protein, 361 aa, Thalictrum flavum
MAVEGKQVAPKKAIIVELLKKLELGLVPDDEIKKLIRIQLGRRLQWGCKSTYEEQIAQLVNLTHSLRQMKIATEVETLDDQMYEVPIDFLKIMNGSNLKGSCCYFKNDSTTLDEAEIAMLELYCERAQIKDGHSVLDLGCGQGALTLYVAQKYKNSRVTAVTNSVSQKEFIEEESRKRNLSNVEVLLADITTHKMPDTYDRILVVELFEHMKNYELLLRKIKEWMAKDGLLFVEHICHKTFAYHYEPIDEDDWFTEYVFPAGTMIIPSASFFLYFQDDVSVVNHWTLSGKHFSRTNEEWLKRLDANVELIKPMFVTITGQCRQEAMKLINYWRGFCLSGMEMFGYNNGEEWMASHVLFKKK

SEQ ID NO: 99 - protein, 358 aa, Coptis japonica
MAVEAKQTKKAAIVELLKQLELGLVPYDDIKQLIRRELARRLQWGYKPTYEEQIAEIQNLTHSLRQMKIATEVETLDSQLYEIPIEFLKIMNGSNLKGSCCYFKEDSTTLDEAEIAMLDLYCERAQIQDGQSVLDLGCGQGALTLHVAQKYKNCRVTAVTNSVSQKEYIEEESRRRNLLNVEVKLADITTHEMAETYDRILVIELFEHMKNYELLLRKISEWISKDGLLFLEHICHKTFAYHYEPLDDDDWFTEYVFPAGTMIIPSASFFLYFQDDVSVVNHWTLSGKHFSRTNEEWLKRLDANLDVIKPMFETLMGNEEEAVKLINYWRGFCLSGMEMFGYNNGEEWMASHVLFKKK

SEQ ID NO: 100 - protein, 351 aa, Papaver somniferum
MQLKAKEELLRNMELGLIPDQEIRQLIRVELEKRLQWGYKETHEEQLSQLLDLVHSLKGMKMATEMENLDLKLYEAPMEFLKIQHGSNMKQSAGYYTDESTTLDEAEIAMLDLYMERAQIKDGQSVLDLGCGLGAVALFGANKFKKCQFTGVTSSVEQKDYIEGKCKELKLTNVKVLLADITTYETEERFDRIFAVELIEHMKNYQLLLKKISEWMKDDGLLFVEHVCHKTLAYHYEPVDAEDWYTNYIFPAGTLTLSSASMLLYFQDDVSVVNQWTLSGKHYSRSHEEWLKNMDKNIVEFKEIMRSITKTEKEAIKLLNFWRIFCMCGAELFGYKNGEEWMLTHLLFKKK

SEQ ID NO: 101 - protein, 358 aa, Papaver somniferum
MGSIDEVKKESAGETLGRLLKGEIKDEELKKLIKFQFEKRLQWGYKSSHQEQLSFNLDFIKSLKKMEMSGEIETMNKETYELPSEFLEAVFGKTVKQSMCYFTHESATIDEAEEAAHELYCERAQIKDGQTVLDIGCGQGGLVLYIAQKYKNCHVTGLTNSKAQVNYLLKQAEKLGLTNVDAILADVTQYESDKTYDRLLMIEAIEHMKNLQLFMKKLSTWMTKESLLFVDHVCHKTFAHFFEAVDEDDWYSGFIFPPGCATILAANSLLYFQDDVSVVDHWVVNGMHMARSVDIWRKALDKNMEAAKEILLPGLGGSHETVNGVVTHIRTFCMGGYEQFSMNNGDEWMVAQLLFKKK

SEQ ID NO: 102 - protein, 350 aa, Eschscholzia californica
MGSSAGEIMGRLMKGEIEDEELKKLIRHQWDRRIEWGYKPTHEKQLAFNLDFIKGLKEMVMSGEIDTMNKETYELPTAFLEAVFGKTVKQSCCYFKDENSTIDEAEEAAHELYCERAQIKDGQTVLDIGCGQGGLVLYIAEKYKNCHVTGLTNSKAQANYIEQQAEKLELTNVDVIFADVTKFDTDKTYDRILVVETIEHMKNIQLFMKKLSTWMTEDSLLFVDHISHKTFNHNFEALDEDDWYSGFIFPKGCVTILSSSTLLYFQDDVSALDHWVVNGMHMARSVEAWRKKLDETIEAAREILEPGLGSKEAVNQVITHIRTFCIGGYEQFSYNNGEEWMITQILFKKK

SEQ ID NO: 103 - protein, 363 aa, Papaver somniferum
MSTTMETTKISQQDDLWKNMELGQISDEEVRRLMKIGIEKRIKWGTKPTQQEQLAQLLDFNKSLRGMKMATEIDTLENHKIYETPESFNQIIGGKESAGLFTDETTTTMEEANTKMMDLYCERAGLKDGHTILDLGCGAGLLVLHLAKKYKKSKITGITNTSSHKEYILKQCKNLNLSNVEIILADVTKVDIESTFDRVFVIGLIEHMKNFELFLRKISKWMKDDGLLLLEHLCHKSFSDHWEPLSEDDWYAKNFFPSGTLVIPSATCLLYFQEDVTVIDHWILSGNNFARSNEVILKRIDGKIEEVKDIFMSFYGIGREEAVKLINWWRLLCITANELFKYNNGEEWLISQLLFKKKLMTCI

SEQ ID NO: 104 - protein, 356 aa, Thalictrum flavum
METKQTKKEAVANLIKRIEHGEVSDEEIRGMMKIQVQKRLKWGYKPTHEQQLAQLVTFAQSLKGMEMAEEVDTLDAELYEIPLPFLHIMCGKTLKFSPGYFKDESTTLDESEVYMMDLYCERAQIKDGQSILDLGCGHGSLTLHVAQKYRGCKVTGITNSVSQKEFIMDQCKKLDLSNVEIILEDVTKFETEITYDRIFAVALIEHMKNYELFLKKVSTWIAQYGLLFVEHHCHKVFAYQYEPLDEDDWYTEYIFPSGTLVMSSSSILLYFQEDVSVVNHWTLSGKHPSLGFKQWLKRLDDNIDEVKEIFESFYGSKEKAMKFITYWRVFCIAHSQMYSTNNGEEWMLSQVLFKKK

SEQ ID NO: 105 - protein, 358 aa, Papaver bracteatum
MGSIDEVKKESAGETLGRLLKGEIKDEELKKLIKFQFEKRLQWGYKSSHQEQLSFNLDFIKSLKKMEMSGEIETMNKETYELPSEFLEAVFGKTVKQSMCYFKHESATIDEAEEAAHELYCERAQIKDGQTVLDIGCGQGGLVLYIARKYKKCHVTGLTNSKAQVNYLLKQAEKLGLTNVDAILADVTQYESDKTYDRLLMIEAIEHMKNLQLFMKKLSTWMTEESLLFVDHVCHKTFAHFFEAVDEDDWYSGFIFPPGCATILAANSLLYFQDDVSVVDHWVVNGMHMARSVDIWRKALDKNMEAAKEILLPGLGGSHEAVNGVVTHIRTFCMGGYEQFSMNDGDEWMVAQLLFKKK

SEQ ID NO: 106 - protein, 358 aa, Papaver bracteatum
MGSIEEVKKESAEETLGRLLRGEINDEELKKLIKYQLEKRLQWGYKSSHQEQLSFNLDFINSLKKMGMSGQVEAFTNEVYELPTECFEAAYGKSMKLSGCYFKHESSTIDEAEEASHELYCERAQIKDGQTVLDIGCGQGGLVLYVAQKYKNCHVTGLTNSKEQVNYILKQAEKLGLRNVDVILADVTQYESDKTYDRILVIGVVEHMKNMQLFIKKLSTWMAEDSLLFVDHSCHKTFNHFFEALDEDDWYSGYIFPPGCATFLSADSLLYFQDDVSVVDHWVVNGMHFARTVDAWRKKLDKNMEAVKEILLPGLGGNHEAVNGVITHIRTCCVGGYVQFSLNDGDEWMNAQLLFKKK

SEQ ID NO: 107 - protein, 368 aa, Argemone mexicana
MCLFFAEKMGLMAEANNQQQLKKEDLLKNMELGLIPDEEIRKLIRVQLEKRLNWGYKSTHEQQLSQLLHLVHSLKKMKIATEMENLDLKLYEAPFSFVQIQHGSTIKESSGLFKDESTTLDEAEIAMLDLYTKRAKIEDGQSVLDLGCGLGAVTLYVAQKFKNCYVTGITSSVEQKDFIEGRCKELKLSNVKVILADITTYETEEKYNRIFAVELIEHMKNYELLLRKISEWMKQDGLLFIEHVCHKTLAYHYEPLDEEDWYTNYIFPAGTLTLSSATLLLYFQDDVAVVDQWTLSGKHYSRSHEEWLKRIDGNIEEVKEIMKSITKSEEEAKKLLNFWRIFCMCGAELFGYKNGEEWMMTHILFKKK

SEQ ID NO: 108 - protein, 364 aa, Glaucium flavum
MDLMATSKQVKKKEELLKNMELGLVPDEEIRRLIRIELEKRLKWGYKPTHQQQLAQLLDLVHSLKKMKIATEMESLDLKLYEAPFSFVQIKHGSTIKESSSYFKDESMTLDEAEIAMLDLYVERAQIEDGQSVLDLGCGLGAVTLHVAKKYKNCHVTGLTNSVEQKDFIEGKCKELNLSNVKVILADVTSHEMEDKFDRIFAVELIEHMKNYELLLRRISKWMKDDGLLFIEHVCHKTFAYHYEPIDEDDWYTEYIFPAGTLTLSSASLLLYFQDDVSVVNHWTLSGKHYSRSHEEWLKRIDGNMDAVKEIMKSITKTEEEAVKLINFWRIFCMCGAELFGYKDGEEWMMSHVLFKKKQLLQQC

SEQ ID NO: 109 - protein, 353 aa, Eschscholzia californica
MVDLKVEKEELLKSMELGLVPDEDIRKHIRSQLEKRLKWGYKPNHEQQLAQLLDVIHSLKKMKISKEYESFDLRLYEAPFDFHKIQLGTHLKESCSYYKDESTTLDEAEGAMLDLYTQKAKIEDGQSILDLGCGVGAVTLFVANKYKNCKVTGITSCQWQKDFIENKCKELNLTNVRVIIGDVTAYEMEETFDRIFAIELIEHMKNYELLLRKISKWMKDDGLLFIEHVCHKILAYPYEPIDEEDWFTEYIFPGGTLTLSSASLLLYFQDDVSVVEHSSLNGKHYSRSHGEWLKNIDANIDEVKGIMRSITKTEEEAVRLVNFWRIFCMCGIELFGYNNGEEWMVSHILLKKK

SEQ ID NO: 110 - protein, 359 aa, Eschscholzia californica
MAADLVVKKWNNKKELIDEMELGLVGDEEIRELIRNDLEKRLKWGYKSNHEQQLAQLLHFVHSLRGMKIAADEVESFNIKVYEAPFSFNKIQLGSSLKESSCYYKHDETTLDEGEIAMMELYTEKAQIKDGQSVLDLGCGLGSLTLYVANKYPNCKVTGTTASLWHKDFIESKCKEQELTNVKIVLGDATTHEMEERFDRILAIGLIEHLKNYGLLLGRISKWLKDDGFLFIQHVCHKTLAYPLVPVDEEDWIGEYIFPGGTLTMPSASLLLYFQDELSVVDHSTLNGKHFSRTHEEWLKNIDAKIDEVKEILKSVTKTEEEVVRLTNFWRIFCMFGVEMFGYNEGEEWMLSQILFKKK

SEQ ID NO: 111 - protein, 353 aa, Chelidonium majus
MASGKVVDLLKRLDSGLVSDEELRRVIRFELERRLKWGYKPTHEQQLAELLNLAHATKQMEIATKIDTLNSTMYEVPNSFLEIQLGSTLKESCLYFKDESTTVDEAEIAMMDLYLERAQIKDGQIILDLGCGLGALAFHIAQKYTNCNVTSVTNSVKQKEFIEEKCKILNVSNVKVILTDICTLEMEATFDRIFAIGLIEHMKNYELLLRKFSAWMKQDGLLFIEHLCHKTLGYHNEPIDEDDWYTAYFFPAGTLTFIPSSFLLYFQDDVSVVNHWTLSGKHFSRSNEEWLKRMDNKIDEVKEIYKAAASETKDDDIMKLIRLWRFLSISAAEMFGYKDGEEWMISQVLFKKK

SEQ ID NO: 112 - protein, 366 aa, Eschscholzia californica
MASLVEEGSFVNNKESVKERVSELVKRLKNGLVSDEELRKLMRVELEKRLEWGYKSTHEQQLSQLIDLAHSMKKMEIAMEIDALNSTVYEVPLSFLQIIHGTTIKESCLYFKDESTTVDEAEIAMMDLYLERAQIKDGQSILDLGCGLGGFSFHIASKFTGCNITAVTNSVKQKEFIEEKCKTLNVPNIKVILADICTTEIENVFDRIIAIGLIEHMKNYELLLKKFSKWMTQDGLLFIEHLCHKTFGYHNEPLDEDDWYTTYFFPAGTLTFIPSSFLLYFQDDVSVVDHWTLNGKHFARSNEEWLKRMDEKMDEVKQIFRSNLKSENEVTKTIGEWRFLSMSAAEMFGYNNGEEWMVSQLLFKKK

SEQ ID NO: 113 - protein, 362 aa, Glaucium flavum
MGSNETNGELKTKEMVPDLLKRLESGLVADEELRKLIRFELERRLKWGYKPTHEQQLAELLKLAHSTKQMKIATETDSLNSTMYEVPIPFLQLQFGSAIKESCCYFKDESTTLDEAEVAMMDLYLERTQIKDGQSILDLGCGLGALAFHIVQKYPNCNVLAITNSVEQKEFIEEKCKIRKVENVKVSLADICTLEMKTTFDRIFAIGLLEHMKNYQLLLKKFSNWMKQDGLLFIEHLCHKTLAYHYEPLDEDDWYTEYFFPAGTLTIISSSFLLYFQDDVSIVNHWSLSGKHFSRSNEEWLKRMDMKIDEVKEILEAAFENKDHDITKLINHWRFLAINATEMFGYNNGEEWMVSQVLFKKK

SEQ ID NO: 114 - protein, 365 aa, Sanguinaria canadensis
MASDHEVSNKELKKKKEVITELLKRLESGLVSDEELRGLIRFELERRLRWGYKPTHEQQLAQLLNLAHSMKQMKIATEIDALNSTMYEVPIPFLQIQLGSTLKESCCYFKDESTTVDEAEIAMMDLYLERAQIKDGQSILDLGCGLGALAFHIAQKYTNCNITAITNSVRQKEFIEEKCKILNVSNVKVSLADICTLEMEATFDRIFAIGLIEHMKNYELLLKKFSEWMKQDGLIFIEHLCHKTLAYHYEPLDEDDWYTEYFFPAGTLTLISSSFLLYFQDDVSVVDHWTLSGKHFSRSNEEWLKRMDEKIDEVKEIFESVSDSKDDDVTKLINHWRFFCISSAEMFGYNNGEEWMISQVLFKKK

SEQ ID NO: 115 - protein, 371 aa, Corydalis cheilanthifolia
MIKKSKIMAFSDHHHEVVKNHSKKEMIADLLKRLEAGLVPDEEMRNLFRFELERRLQWGYKSIHQEQLSQLLKLAHSTKEMTIVAEMDALNSSMYELPISFLQIQLGSNLKQSSLYFKDELTTVDEAEVAIMDLYLERAQIEDGQSILDLGCGLGAFSFHVARKYTNCNITAVTNSLTQKEFIEKKSKILNIQNVKVIFADVTTVEMETTFDRVFAIGLIEHMQNYELFLKKLSKWMKQDGLLFIEHFCHKTLAYHYKPIDEDDWFTNLLYPNGTVISSSLLLYFQDDVSVVDHWSLSGKHFSRASEESLKRMDAKMDEMKEIFESITDSKEEAMKLINQWRIFCISCAEMFGYNNGEEWMTSHFLFKKKL

SEQ ID NO: 116 - protein, 373 aa, Corydalis cheilanthifolia
MGSSTASDHEMVIMENDSKNKQVVIADLLKRLVGGLVPDEEMRNMFRFELEKRLKWGYKSTHQQQLSQLLNLVELNKGIAKIAPEMDALNSAMYEVPIPYLKLMLGSTLKQSCLYFKDESTTLDEAEIEMMDLYLERADIQDGQSILDLGCGLGGLGFHIAQKYISCNITALTNSLTQKEFIEEKCKTLNIPNVKVILADVTTVEIETTFDRLFAIGLVEHMENYELFLRKLSKWMKQDGLLFIEHLCHKTLAYHYKPIDEDDWYSNLLYPTGTLTSASFLLYFQDDLSVVDHWSLSGKHFSRATEEWLKMIDANMDKIREIYESVTESKEEATRSINQWRIFCISCAEMFGYNDGEEWMISHFLFKNKKQIE

SEQ ID NO: 117 - protein, 366 aa, Corydalis cheilanthifolia
MATSDQEVKTSKMEMIADLLKRLEAGLVPDDEIRSLIRVELERRLKWGYKSTHQEQLDQLLNLAHSIKKMKIASTEMDGLTSTMYEVPISLVQIQLGSHLKESCLYFKDETTTVDEAEIAMMDLYLERAQIKDGQSILDLGCGLGAVSFHIAQKYTSCNITAVTNSVRQKEFIEEKSKTLNVPNVKVLLADITTLEMEHTFDRLFAISLIEHMENYELLLRKLSEWMKQDGLLFIEHLCHKTLSYHFEPMDEDDWYTNLLFPAGTLTLVSASFLLYFQDDLSVVNQWVMSGKHFSRANEEWLKNMDAKMDEMREIFESITDSEEEVVKLINHWRIFCISSAEMFAYNDGEEWMNSHVLFKKKKQIQ

SEQ ID NO: 118 - protein, 359 aa, Corydalis cheilanthifolia
MAGSGANKEMIADLLKRLEVGLVPDEEIRSLIRFQLKRRLKWGYKTTHQEQLEQLLSLAHSIRKMKIATEMDALNSTMYEVPISFMQIVFGSTLKESCLYFKDEATTVNEAEIAMMDLYLERAQIKDGQSILDLGCGMGSLCFHIARKYTNCNITAVTNSVSQKEFIEEKSKTLNLPNVKVILADITTLEMDDTYDCLFAIGLIEHMKNYELLLRKLSNWMKQDSLLFIDHVCHKTLAYHYEPIDEDDWYTNLLFPAGTLTLVSASFLLYFQDDLSLVDHWSMSGKHFSRTNKEWLKNIDGKMDKIREIVKSITDSEEEVVKLINHWRMLCINSSEMFGFNDGEEWMNSHVLFKKKKQI

SEQ ID NO: 119 - protein, 353 aa, Sanguinaria canadensis
MEMIADLLKRLEAGLVPDDEIRSLIRVELERRLKWGYKSTHQEQLDQLLNLAHSIKKMKIASTEMDGLTSTMYEVPISLVQIQLGSHLKESCLYFKDETTTVDEAEIAMMDLYLERAQIKDGQSILDLGCGLGSVCFHIARKYTSCNITAVTNSVSQKEFIEEKSKTLNVPNVKVLLADITTLEMDDTFDCLFAIGLIEHMENYELLLRKLSDWMKQDGLLFIDHVCHKTLSYHFEPMDEDDWYTNLLFPAGTLTLVSASFLLYFQDDLSLVDHWSMSGKHFSRTNKEWLKNIDGKMDKIREIVKSITDSEEEVVKLINHWRMLCINSSEMFGFNDGEEWMNSHVLFKKKKQI

SEQ ID NO: 120 - protein, 363 aa, Papaver bracteatum
MCTTMDTTKISQQDDLWKNMELGLISDEEVRRLMKIETEKRIKWGTKPTQQEQLAQLLDFNKSLRGMKMATEVHALENHKIYEIPDSFNQIIGGKESAGLFTDEATTTIEEANTKMMDLYCERAGLKDGQTILDIGCGAGLLVLHLAKKYKNCKITGVTNTSWHKEHILEQCKNLNLSNVEVILADVTTVDIERTFDRVFVIGLIEHMKNFELFLRKISKWMKDDGLLFLEHLCHKSFSDHWEPLSEDDWYAKNFFPSGTLVIPSATCLLYFQEDVTVKDHWLLSGNNFARSNEAILKRIDSKIEEVKDIFMSFYGIGEEEAVKLINWWRLLCITANELFKYNNGEEWLISQLLFKKKLMTCI

SEQ ID NO: 121 - protein, 366 aa, Papaver bracteatum
MVKGDQFQTTTMEETKISQENDLWTNMELGLIPDEEVRRLMKIEIEKRIEWGMKPTQHQQLAQLLDFTKSLRGMKMATELDKLDSKLYETPHSFNQIVNGSTLKESSGLYTDVTTTMDEASIKMMDLYCERANIKDGQTILDLGCGPGPLVLHIAKKYSNCKITGVTNAFSQREYILEECKKLSLSNVEIILADVTSLDLETTFDRVFVIGFIEHMKNFELFLRKISKWMKDDAVLFLEHFCHKSFSYHGEPLSEDDWYAKNFFAPGTLVIPSATCLLYFQEDLAVIDHWFLSGNHFARTNEEMLKGIDGKIEEIKDIFMSFYGINEAEAVKLINWWRLFCITGAEMFSYNNGEEWFISQLLFKKK

SEQ ID NO: 122 - protein, 364 aa, Eschscholzia californica
MALEQEDSMSVPERNEGVADLIKRMELGLVNDEEIRRLMRIQIENRLKWGYKPTHDQQLAQHLHFINSLKEMKMATEMDSLDSQVYESPNSFQQIMCGRSMKESAGLFMDDVTTVEEAHIRMMDLYCDKATFEDGQKILDLGCGHGSVVLHVAQKYKGCQVTGVTNSSAQKQYILEQCKKLDLSNVEIILADVTTLEMEEKFDRVIIIGLIEHMKNFKLFFQKVSKWMKEGGLLFLENYFHKDFAYHCEKIDEDDWYDGYIFPPGSLLMPSASTLLYFQEDLTVADHWVLPGTHFAKTFEEFLKKIDLRIEEVREIFEAFYGISKEEAMKLSNYWRNFCISAMEIFNYNNGQEWMISHLLYTKK

SEQ ID NO: 123 - protein, 368 aa, Chelidonium majus
METGKNNQNMKTTIDDLWNQMMLGIVPDKEIRRLMKIELKKRLDWGYRPTHQQQLSQLLDFAKGLCNYCWTALRCMKMSAEFDTLDSKVYETPKSFQQIMCGTTIKESSGLFMNESTTLDQAQISMLDLYFDKAKIKDGQSILDLGCGHGALILYLAQKYQNCNITGVTNSLSQKEFIVEKCKKLGLSNVEILLADVTKLEMEDMFDRVFVIGLIEHMKNFELFLRKISEWMKPDGLLFLEHYCHKSFAHQWEPIDEEDWFSKYIFPPGTVIIPSASFLLYFQEDVKVIDHWTLSGNHFARTQEEWLKGIDGHIDEVEKTFESFYGISKEEAVKLINFWRVFCLSGVEMFGYNNGEEWMISHLLFKKK

SEQ ID NO: 124 - protein, 361 aa, Glaucium flavum
MTMEANNAKKEAIENLWEQMMMGLVPDHEITRLMKSELQKRLNWGYKPTHQQQISQLLDFAKSLRRMEMSLDFDNLELDTKMYETPESFQLIMSGTTLKESSGLFTDETATLDQTQIRMMDLYLEKAKIKDGQSILDLGCGHGALILHVAQKYRNCNVTGVTNSIAQKEFIFKQCKKLGLSNVEMVLADVTKCEMKATFDHIFVIGLIEHMKNFELFLRKVSEWMKSDGLLFMEHYCHKSFAYQWEPMDDDDLFSKYVFPPGSAIIPSASFLLYFQDDLTVVDHWTLSGNHFARTHQEWLKRIDSQSDEIKGIFESFYGISKEEAVKLINYWRVFCLFGVEMFGYNNGEEWMISHLLFKKK

SEQ ID NO: 125 - protein, 363 aa, Corydalis cheilanthifolia
MEVVATSSARNPKKEIVDLWKRMELGLIPDEEIRDLMKIGLEKRLKWGYKPTHEQQLSQLLHFAKSLRSMKMASEMETLDDQMYETPTAFQQLMCGSTIKESAGFFKDESTTLDEAEIKMLDLYCEKARIEDGQKILDLGCGHGAVMLHIAQKYKNCNVTGVTNSISQQQFIVQRSKELNLSNVNMILADVTMLEMDATYDRIFIIGLIEHMKNFELFLRKISKWITKEGLLFLEHYCHKTFAYQCEPVDEDDWYNMFIFPPGTLILPSASFLLYFQDDLIVVDRWTLNGNHYARTQEEWLKRIDANVDGVKQMFESVCDGNKEEAVKLMNFWRIFCISGAEMLAYNNGEEWMISHYLFKKRN

SEQ ID NO: 126 - protein, 357 aa, Nigella sativa
MEATQITKKQGVAELIKRIENGQVPDEEITRMMKIQIQKRLKLGYKSTHEQQLAQLLHFVHSLQKMEMAEEVDTLDSELYEIPLPFLHIMCGKALKFSPGYFKDESTTLDESEVNMLDLYCERAQIEDGQTILDLGCGHGSLTLHVAKKYRGCKVTGITNSVSQKDFIMEECKKLNLSNVEIILEDVTKFETGTTYDRIFAVALIEHMKNYELFLKKVSAWMAQDGLLFVEHHCHKVFAYKYEPIDDDDWYTEYIFPTGTLVMSSSSILLYFQEDVSVVNHWTLSGKHPSLGFKQWLKRIDDNIDEIKEIFESFYGSKEKATKFITYWRVFCIAHSEMYATNGGEEWMLSQVLFKRK

SEQ ID NO: 127 - protein, 349 aa, Sanguinaria canadensis
MGGVADLLKKMELGLVPEEEIRRLMRIIIEKRLEWGYKPTHAEQLDHLTNFIQCLRGMKMADEIDALDAKMYEIPLPFMQTICGSTLKFSPGYFKDESTTLDESEIHMMDLYCERAEVKDGHSILDLGCGHGGFVLHVAQKYKNSIVTGVTNSVAEKEFIMTQCKKLCLSNVEIILADVTKFEPETTYDRVFAIALIEHMKNYELVLEKLSKWVAQDGFLFVEHHCHKVFPYKYEPLDEDDWYTEYIFPGGTIVLPSASILLYFQKDVSVVNHWSLNGKHPARGFKEWLKRLDENMDAVKAIFEPFYGSKEEAMKWITYWRVFCITHSEMYAYNNGEEWMLSQVLFKRK

SEQ ID NO: 128 - protein, 350 aa, Jeffersonia diphylla
MSKGVAKLVERMELGLVSDDEVRRLMRILIEKRLKWGYKPTHEEQLTYLTNFIQGLKGMKIAEEIDALDAKMYEIPIAFMQILCGYSLKFSPGFFEDESTTLDESETIMMDLYCERAQVQDGQSILDLGCGHGGFVLHVAQKYKNCKVTGVTNSVSETEYIMEQCKKLGLSNVEIIIADVTKFEPEVTYDRVFAIALIEHMKNYELVLQKLSKWVAQDGFLFVDHHCHKVFPYKYEPIDEDDWYTQYIFPGGTLVLPSASILLYFQEDVSIVNHWTLSGNHPARGFKEWLKRLDDNMDEIKAIFEPFYGSKEEAMKWITYWRVFCITHSEMYAYNGGEEWMISQVLFKRK

SEQ ID NO: 129 - protein, 357 aa, Berberis thunbergii
MEVKQAGKEGVTELLVKRMELGLVPEEEIRRLMRIQIQKRLDWGYKPTHEEQLAHLTKFIQNIRGMKMADEIDALDAKMYEIPLPFLQTICGKTLKFSPGYFKDESTTLDESETLMMDLYCERAQVKDGQSILDLGCGHGGFVLHLAQKYRNSVVTGVTNSVSETEYIKEQCKKLGLSNVEIIIADVTKFEPEVTYDRVFAIALIEHMKNYALVLNKISKWVAQDGYLFVEHHCHKVFPYKYEPLDEDDWYTNYIFPGGTLILPSASILLYFQEDVTVLNHWSLSGKHPSRGFIEWLKRLDENIDVIMGIFEPFYGSKEEATKWINYWRVFCMTHSEMYAYGNGEEWMLSQVLLKRK

SEQ ID NO: 130 - protein, 339 aa, Berberis aquifolium
MELGLVPEKEIRRLMRIQIQKRLEWGYKPTHEEQLAHLTKFIQNIRGMKMADEIDALDAKMYEIPLPFLQTICGKTLKFSPGYFKDESTTLDESETLMMDLYCERAQVKDGQSILDLGCGHGGFVLHLAQKYRNSIVTGVTNSVSETEYIKEQCKKLGLSNVEIIIADVTKFEPEVTYDRVFAIALIEHMKNYALVLNKISKWVAQDGYLFVEHHCHKVFPYKYEPLDEDDWYTNYIFPGGTLILPSASILLYFQEDVTVLNHWSLSGKHPSRGFIEWLKRLDENIDVIMGIFEPFYGSKEEATKWINYWRVFCITHSEMYAYGNGEEWMLSQVLLKRK

SEQ ID NO: 131 - protein, 355 aa, Menispermum canadense
  VARIANT 86: note = Any amino acid
  VARIANT 96: note = Any amino acid
  VARIANT 127: note = Any amino acid
  VARIANT 138: note = Any amino acid
  VARIANT 153: note = Any amino acid
  VARIANT 292: note = Any amino acid
MDKANERELKRAELFKKLEDDLVTYDEIKQVMRTELAKRLEWGYKPTHQQQLAHLLDFAHALEGMKIANEVETLASEVYETPLPFXEIVLGPAKKXSSCLFEDESTTLEQAEIAMLDLYFERAQIRXGMSVLDLGCGXGSVGLHIARKYKNCXVTCITNSISQKQYIENQCKLYNLSNVKIILADIVAHDTDDTFDVVLVIGVIEHMKNYALLLNKISKWMAKDGLLFVEHLCHKTFPYHFEPLDEDDWYSNFVFPTGTLTMPSVSFLLYFQADVSILNHWILSGKNFSRTXEEFLKRIDANVDAIKDGLKPSLGSEGVAKLISYWRGFCLTGMEMFGYNNGEEWMVSQVLFKNK

SEQ ID NO: 132 - protein, 366 aa, Tinospora cordifolia
MEDNNNLLQEEMNVVELLQRPELGLVPDEKIRKLTRLQLQKRLKWGYKPTHEAQLSHLFQFIHSLPSLNMESEDENPKSWLYETPTSFLQLLYGDCIKESDTYYKEDTATLEEAVINMLELYCERARITEGLSVLDLGCGYGALTLHVAQKYKSCKVTGVTSSISQKQYIMEKCKKLNLTNVEIILADVATIEIEAASYDRIFALGIFEHVNDYKLFLGKLSKWMKQDGLLFVEYLCHKTFPYQNKPLDKGDKWYNEYVFPSGGLIIPSASFILYFQNDVSVVRQWTQGGQHSARTFEELLKRIDGNIDKIKEIFIESYGSKEDAVRFINYWRVFLITGVEMFSYNDGEEWMGAHFLFKKKFIMQE

SEQ ID NO: 133 - protein, 359 aa, Cissampelos pareira
  source 1..359: note = subspecies: mucronata
MEVKQSKGDELRSRVAELLERPELGLVPDEEIRRLAKARLEKRLKWGYKATHGEQLSSLLQFVESLPSLNMASEDDSPKAWLYETPTSFLQLIYGDIIKESGSYYKDESTTLEEAMIHNMNLCCERANIKEGQSVVDLGCGYGAFILHVAQKYKTCRVTGITSSISQKHYIMEQCKKLNLSNVEVILADVATIKLDATFDRVFAAGMFEHVNDYKSFLRKITNWMKPDGRLFVEHLCNKTFPYQNKPLDDGDNWGEYVFPSGGLIIPSASLLLYFQEDVSIVNHWTFSGKHAANKFEELLKRIDAKIDAIKRIFNECYGSKDSIRFINYWRVFLITAAEMFGYNNGEEWMGVHLLFKKK

SEQ ID NO: 134 - protein, 352 aa, Cocculus orbiculatus
GLKSSVAELLERPELGLVPDGEIRKLTKTRLAKRLEWGYKATHEDQLSHLLRFIHSLPSLNMASEDDSPKAWLYETPTSFLQLIYGDIIKESGTYYKDESSTLEEAIIHNMDLCCERARIKEGQSVLDLGCGYGAFTLHVAQKYKSCSVTGITSSISQKDYIMEQCKKLNLSNVEVILADVATIKMNTTFDRVFALGMFEHINDYKLFLRRISNWMKHDGLLFVEHLCNKTFAYQNKPLDDGDDWFNEYVFPSAGLIIPSASLLLYFQEDVSIVHHWTFSGKHAAYKFEELLERIDAKIEAIKEIFIECYGSKEDAIRFINYWRVFLITAAEMFAYRDGEEWMGSHVLFKKK

SEQ ID NO: 135 - protein, 364 aa, Cissampelos pareira
  source 1..364: note = subspecies: mucronata
MEAKQHESNNNIDEELKNRVNIGEQEERPGFEDEEIRRLAKAQLAKRLKWGYKPTHEQQLSHLLQFLQSLPSLNMASEDESSKAWLYETPTSFLQLLFGNVIKFSGYYYKHESSTFEESMIHNMDLCCERANIKEGQNVIDLGCGYGAFVLHVAQKYKSCSVTGITCSITQKHHIMEECKKLNLCNVKVILADVATIELGTAFDRVFAFGMFEEINDYKLILRKISNWMKPDGLFFVEHLCHKTLAYQNKLIDDQDWYEEYIFPSGGLIVPSASLLLYFQDDLSVVYHWTYNGKHGARSFEKMLERTDANIDTIKDMFTEFYGSKEKAIKFINYWRVFFITAAEMFAYNDGEEWMCSQLLFKKK

SEQ ID NO: 136 - protein, 364 aa, Cissampelos pareira
  source 1..364: note = subspecies: mucronata
MEHKIEDIRKLKSRVEEQLERPELGLVKDEDIKTLAKAKLEKRLKWGYKPTYAEQLSNLLQFAQSLPSLKMENVDDQGSSKQWLYGVPSEFLQIIYGGIIKMSGSYYEDESTTLEESMIKDMDSCCEKANVKEGHSVLDIGCGYGSLIIHIAKKYRTCNVTGITNFVEQKQYIMEECKKLNLSNVEVIVGDGTTINLNTTTFDRVFVTGMLEEINDYKLFLKSVSDWMKPDGLLLVTHFCHKTFAYQNNKALDDEDWHNEYIFPSGNLIVPSASLLLYFQEDLSVVSHWATNGTHTGRTCKKLVERIDANIEKIKEIFSEFYGSKEDAIRMINYWRVLCITGAEMYTCKDGEEWMDVYYLFKKK

SEQ ID NO: 137 - protein, 1049 aa, synthetic construct
  REGION 1..1049: note = Description of Artificial Sequence: Synthetic polypeptide
MTIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETATLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 138 - protein, 1049 aa, synthetic construct
  REGION 1..1049: note = Description of Artificial Sequence: Synthetic polypeptide
MTIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAADEVMNKLQRANPDDPAYDENKRQFQEDIKVMNDLVDKIIADRKARGEQSDDLLTQMLNGKDPETGEPLDDGNIRYQIIAFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETATLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 139 - protein, 1049 aa, synthetic construct; identical to SEQ ID NO: 83
  REGION 1..1049: note = Description of Artificial Sequence: Synthetic polypeptide

SEQ ID NO: 140 - protein, 1049 aa, synthetic construct
  REGION 1..1049: note = Description of Artificial Sequence: Synthetic polypeptide
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISAVRAADEVMNKLQRANPDDPAYDENKRQFQEDIKVMNDLVDKIIADRKARGEQSDDLLTQMLNGKDPETGEPLDDGNIRYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 141 - protein, 1049 aa, synthetic construct
  REGION 1..1049: note = Description of Artificial Sequence: Synthetic polypeptide
MTIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISAVRAADEVMNKLQRANPDDPAYDENKRQFQEDIKVMNDLVDKIIADRKARGEQSDDLLTQMLNGKDPETGEPLDDGNIRYQIIAFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRACIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 142 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctttgaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctatggttagagctttggatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcaccttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccgctaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 143 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctttgaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctatggttagagctgcagatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcgctttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccgctaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 144 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctgctaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctatggttagagctgcagatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcaccttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccttgaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 145 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctgctaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctgctgttagagctgcagatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcaccttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccttgaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 146 - DNA, 3150 bp, synthetic construct
  misc_feature 1..3150: note = Description of Artificial Sequence: Synthetic polynucleotide
  source 1..3150: mol_type = other DNA
atgaccatcaaagaaatgccacaacctaagactttcggtgaattgaagaatttgcctttgttgaacaccgataagccagttcaagctttgatgaagattgctgatgaattgggtgaaatcttcaagtttgaagctccaggtagagtcactagatacttgtcatctcaaagattgatcaaagaagcctgcgacgaatccagatttgataagaatttgtctcaagctgctaagttcgctagagattttgctggtgatggtttggttacttcttggactcacgaaaagaattggaagaaggcccataacattttgttgccatctttctcacaacaagccatgaagggttatcatgctatgatggttgatatcgccgttcaattggttcaaaagtgggaaagattgaacgccgatgaacatatcgaagtctctgaagatatgaccagattgaccttggataccattggtttgtgtggtttcaactacagattcaactccttctacagagatcaaccacatccattcatcatctctgctgttagagctgcagatgaagtcatgaacaaattgcaaagagctaatccagacgatccagcttatgacgaaaacaagagacaattccaagaagatatcaaggtcatgaacgatttggtcgataagattatcgctgatagaaaggctagaggtgaacaatctgatgatttgttgacccaaatgttgaacggtaaggatccagaaactggtgaaccattggatgatggtaacatcagataccaaattatcgctttcttgattgctggtcacgaaactacatctggtttgttgtcttttgccttgtactttttggttaagaacccacacgtcttgcaaaaggttgctgaagaagctgcaagagttttggttgatccagttccatcttacaagcaagtcaagcaattgaagtacgttggtatggttttgaacgaagctttgagattgtggccaactgctccagctttttcattatacgctaaagaagataccgtcttgggtggtgaatatccattggaaaaaggtgatgaagttatggtcttgatcccacaattgcatagagataagactgtttggggtgatgatgtcgaagaattcagaccagaaagattcgaaaacccatctgctattccacaacatgcttttaagccatttggtaacggtcaaagagcttgcattggtcaacaattcgctttacatgaagctaccttggttttgggtatgatgttgaaacacttcgacttcgaagatcacaccaactacgaattggatatcaaagaaaccttgaccttgaagccaaagggttttgttgttaaggctaagtccaaaaagattccattgggtggtattccatctccatctactgaacaatccgctaagaaggttagaaagaaagctgaaaacgctcataacacacctttgttggtcttgtacggttctaatatgggtactgctgaaggtacagcaagagatttggcagatattgctatgtctaaaggtttcgctccacaagttgctactttggattctcatgctggtaatttgccaagagaaggtgctgttttgatagttactgcttcttacaatggtcacccaccagataatgctaagcaattcgttgattggttggatcaagcttcagctgatgaagtaaaaggtgttagatactctgttttcggttgcggtgacaaaaattgggctactacttatcaaaaggttccagcctttattgacgaaactttggctgctaaaggtgctgaaaacattgctgacagaggtgaagctgatgcctccgacgacttcgaaggtacttacgaagaatggagagaacacatgtggtctgacgttgctgcttacttcaacttggacatcgaaaactctgaagacaacaagtccactttgtctttgcaattcgttgactccgctgctgacatgccattggctaagatgcacggtgctttctctaccaacgtcgttgcctccaaggaattgcaacaaccaggttctgctagatctactagacacttggaaatcgaattgccaaaggaagcttcctaccaagaaggtgaccacttgggcgttattccaagaaactacgaaggtatcgtcaacagagttactgctagattcggtttggatgcttctcaacaaatcagattagaagctgaagaagaaaagttggctcacttgccattagctaagactgtctccgttgaagaattgttgcaatacgtcgaattgcaagacccagttaccagaacccaattgagagccatggctgccaagaccgtctgtccaccacacaaggttgaattggaagccttgttggaaaagcaagcctacaaggaacaagttttggctaagagattgaccatgttggaattgttggaaaagtacccagcctgcgaaatgaagttctctgaatttatcgccttgttgccatctatcagaccacgttactactctatttcttcctctccacgtgttgacgaaaagcaagcttctattactgtttccgttgtctccggtgaagcttggtccggttacggtgaatacaagggtattgcttctaactacttggctgaattgcaagaaggtgacaccattacttgtttcatctctactccacaatccgaatttactttgccaaaggacccagaaactccattgatcatggttggtccaggtactggtgtcgctccattcagaggtttcgttcaagctagaaaacaattgaaggaacaaggtcaatctttgggtgaagctcacttgtacttcggttgtagatctccacacgaagactacttataccaagaagaattggaaaacgctcaatccgaaggtattatcactttgcacaccgctttctccagaatgccaaaccaaccaaagacttacgtccaacacgttatggaacaagacggtaagaagttgattgaattgttggaccaaggtgctcacttctacatttgtggtgatggttctcaaatggctccagccgttgaagccactttgatgaagtcttacgctgatgttcaccaagtttccgaagccgatgctagattatggttgcaacaattggaagaaaaaggtcgttacgctaaggatgtctgggccggttga

SEQ ID NO: 147 - DNA, 5372 bp, synthetic construct
  misc_feature 1..5372: note = Description of Artificial Sequence: Synthetic polynucleotide
  source 1..5372: mol_type = other DNA
cctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagtggagccaatcaattcttgcggtcaactttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgattcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgc

SEQ ID NO: 148 - DNA, 4977 bp, synthetic construct
  misc_feature 1..4977: note = Description of Artificial Sequence: Synthetic polynucleotide
  source 1..4977: mol_type = other DNA
aacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgccctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagtggagccaatcaattcttgcggtcaactttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgagagcgttggttggtggatcaagcccacgcgtaggcaatcctcgagcagatccgccaggcgtgtatatatagcgtggatggccaggcaactttagtgctgacacatacaggcatatatatatgtgtgcgacaacacatgatcatatggcatgcatgtgctctgtatgtatataaaactcttgttttcttcttttctctaaatattctttccttatacattaggacctttgcagcataaattactatacttctatagacacacaaacacaaatacacacactaaattaataacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcg

SEQ ID NO: 149 - DNA, 5079 bp, synthetic construct
  misc_feature 1..5079: note = Description of Artificial Sequence: Synthetic polynucleotide
  source 1..5079: mol_type = other DNA
acgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcagtggaacgtgcattatgaattagttacgctagggataacagggtaatatagaacccgaacgaccgagcgcagcggcggccgcgctgataccgccgccctcgccgcagttaattaaagtcagtgagcgaggaagcgcgtaactataacggtcctaaggtagcgaatcctgatgcggtattttctccttacgcatctgtgcggtatttcacaccgcatagatcggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagtggagccaatcaattcttgcggtcaactttggacgatatcaatgccgtaatcattgaccagagccaaaacatcctccttaagttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcttaatcggcaaaaaaagaaaagctccggatcaagattgtacgtaaggtgacaagctatttttcaataaagaatatcttccactactgccatctggcgtcataactgcaaagtacacatatattacgatgctgttctattaaatgcttcctatattatatatatagtaatgtcgtgatctatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaaggtgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggacgcgtagtctagaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgccatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccggctgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagcattcaaggcgccttgattatttgacgtggtttgatggcctccacgcacgttgtgatatgtagatgactcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaaacaggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacattatactgaaaaccttgcttgagaaggttttgggacgctcgaaggctttaatttgtaatcattatcactttacgggtcctttccggtgatccgacaggttacggggcggcgacctcgcgggttttcgctatttatgaaaattttccggtttaaggcgtttccgttcttcttcgtcataacttaatgtttttatttaaaatacctcgcgagtggcaacactgaaaatacccatggagcggcgtaaccgtcgcacaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcga

SEQ ID NO: 150 - protein, 21 aa, Papaver rhoeas
TQGLMSYKVVPLDILLTRRML

SEQ ID NO: 151 - protein, 60 aa, synthetic construct
  REGION 1..60: note = Description of Artificial Sequence: Synthetic polypeptide
TPGLMSYKVVPLDILLTHRRIKSCVQLASSERDMESSGVPVITLSSGKVMPVLGMGTFEK

SEQ ID NO: 152 - protein, 27 aa, Papaver rhoeas
MDSSGVPVIPLSSGKGMPALALGTFET

SEQ ID NO: 153 - protein, 900 aa, synthetic construct
  REGION 1..900: note = Description of Artificial Sequence: Synthetic polypeptide
MELQYISYFQPTSSVVALLLALVSILSSVVVLRKTFLNNYSSSPASSTKTAVLSHQRQQSCALPISGLLHIFMNKNGLIHVTLGNMADKYGPIFSFPTGSHRTLVVSSWEMVKECFTGNNDTAFSNRPIPLAFKTIFYACGGIDSYGLSSVPYGKYWRELRKVCVHNLLSNQQLLKFRHLIISQVDTSFNKLYELCKNSEDNQGNYPTTTTAAGMVRIDDWLAELSFNVIGRIVCGFQSGPKTGAPSRVEQFKEAINEASYFMSTSPVSDNVPMLGWIDQLTGLTRNMKHCGKKLDLVVESIINDHRQKRRFSRTKGGDEKDDEQDDFIDICLSIMEQPQLPGNNNPSQIPIKSIVLDMIGGGTDTTKLTTIWTLSLLLNNPHVLDKAKQEVDAHFRTKRRSTNDAAAAVVDFDDIRNLVYIQAIIKESMRLYPASPVVERLSGEDCVVGGFHVPAGTRLWANVWKMQRDPKVWDDPLVFRPDRFLSDEQKMVDVRGQNYELLPFGAGRRVCPGVSFSLDLMQLVLTRLILEFEMKSPSGKVDMTATPGLMSYKVIPLDILLTHRRIKPCVQSAASERDMESSGVPVITLGSGKVMPVLGMGTFEKVGKGSERERLAILKAIEVGYRYFDTAAAYETEEVLGEAIAEALQLGLVKSRDELFISSMLWCTDAHADRVLLALQNSLRNLKLEYVDLYMLPFPASLKPGKITMDIPEEDICRMDYRSVWAAMEECQNLGFTKSIGVSNFSCKKLQELMATANIPPAVNQVEMSPAFQQKKLREYCNANNILVSAISVLGSNGTPWGSNAVLGSEVLKKIAMAKGKSVAQVSMRWVYEQGASLVVKSFSEERLRENLNIFDWELTKEDHEKIGEIPQCRILSAYFLVSPNGPFKSQEELWDDEA